(19) United States
(12) Patent Application Publication
(10) Pub. No.: US 2023/0099010 A1
(43) Pub. Date: Mar. 30, 2023

(54) COMBINATION THERAPY

(71) Applicants: ADC THERAPEUTICS SA, Epalinges (CH); MEDIMMUNE LIMITED, Cambridge, Cambridgeshire (GB)

(72) Inventors: Patricius Hendrikus Cornelis VAN BERKEL, Epalinges (CH); Francesca ZAMMARCHI, Epalinges (CH); Jens WUERTHNER, Epalinges (CH)

(21) Appl. No.: 17/780,154

(22) PCT Filed: Nov. 6, 2020
(86) PCT No.: PCT/EP2020/081361

§ 371 Date: May 26, 2022

(30) Foreign Application Priority Data
Nov. 27, 2019 (GB) 1917254.3

(51) Int. Cl. A61K 47/68; A61K 39/395; A61P 35/00; A61N 5/10

(52) U.S. Cl. CPC A61K 47/6849; A61K 39/3955; A61K 47/6803; A61P 35/00; A61N 5/10; A61N 2005/1098

(57) ABSTRACT

The present disclosure relates to combination therapies for the treatment of pathological conditions, such as cancer. In particular, the present disclosure relates to combination therapies comprising treatment with Antibody Drug Conjugates (ADCs) ADCs which bind to CD25 (CD25-ADCs) and radiotherapy.

### EARLIER APPLICATION

This application claims priority from United Kingdom application GB1917254.3 filed on 27 Nov. 2019.

### FIELD

The present disclosure relates to combination therapies for the treatment of pathological conditions, such as cancer. In particular, the present disclosure relates to combination therapies comprising treatment with Antibody Drug Conjugates (ADCs) ADCs which bind to CD25 (CD25-ADCs) and radiotherapy.

### BACKGROUND

Antibody Therapy Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

CD25

The type I transmembrane protein CD25 is present on activated T- and B-cells, some thymocytes, myeloid precursors, and oligodendrocytes. On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin' lymphomas of mixed cellularity type. The general expression levels are reported to be lower than in tumor infiltrating lymphocytes (TILs), which may result in problems demonstrating CD25 tumor cells in these cases (Levi et al., Merz et al, 1995).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

Therapeutic Uses of Anti-CD25 ADCs

The efficacy of an Antibody Drug Conjugate comprising an anti-CD25 antibody (an anti CD25-ADC) in the treatment of, for example, cancer has been established—see, for example, WO2014/057119, WO2016/083468, and WO2016/166341.

Research continues to further improve the efficacy, tolerability, and clinical utility of anti-CD25 ADCs.

Radiotherapy

Radiotherapy (also known as Radiation therapy) is an important method of cancer treatment. Approximately 50% of all cancer patients will receive radiation therapy during their course of illness [Delaney at al. Cancer 2005; 104: 1129-1137] with an estimation that radiation therapy contributes to around 40% towards curative treatment [Barnett et al., Nat Rev Cancer 2009; 9: 134-142] whilst only accounting for about 5% of the total cost of cancer care [Ringborg et al., Acta Oncol. 2003; 42: 357-365].

Radiotherapy exerts its effects on biological tissue through physical interactions between high energy radiation and biological molecules. High-energy radiation damages genetic material (deoxyribonucleic acid, DNA) of cells both directly and indirectly (via free radicals), blocking the cells' ability to divide and proliferate further [Jackson et al., Nature 2009; 461: 1071-1078]. However, radiotherapy damages both normal cells as well as cancer cells, making it important to maximize the radiation dose to abnormal cancer cells while minimizing exposure to adjacent normal cells and tissues. This differential cancer cell killing effect is accentuated by the fact that cancer cells, in general, are not as efficient as normal cells in repairing the damage caused by radiation treatment [Barnett et al., Nat Rev Cancer 2009; 9: 134-142].

Despite precautions taken to minimize radiation exposure of normal tissues by, for example, increasingly sophisticated targeting and delivery apparatus [Bernier et al., Nature 2004; 4: 737-747] some degree of healthy tissue exposure is typical. Indeed, damage of normal cells surrounding the malignant tumor remains a major limitation on radiotherapy efficacy (Barber et al., 2000, Radiotherapy and Oncology, 55(2), 179-186; Sprung et al. 2005, Clinical Cancer Research, 11(17), 6352-6358). Chao, Leong, & McKay, 2005). This limitation is particularly problematic in the 10-20% with significant radiosensitivity [Schuster, B. et al., BMC Geriatr. 18, 105 (2018) doi:10.1186/s12877-018-0799-y].

In view of these limitations of conventional radiotherapy, a number of strategies have been reported to mitigate the inherent risks. These strategies include using radiosensitizers such as hyperbaric oxygen, carbogen, nicotinamide, metronidazole, hypoxic cell cytotoxic agents including mitomycin-c, tirapazamine, motexafin gadolinium, taxanes, and/or irinotecan to increase the efficacy of radiotherapy (Lu et al., 2016, BioMed Research International, Volume 2016, Article ID 4376720, 5 pages). An alternative strategy is to employ antioxidative radioprotectors before or at the time of radiotherapy to protect healthy cells. (Prasanna et al., 2015, Radiation Research, 184(3), 235-248). Notwithstanding these strategies, research continues to identify low-toxicity and high-efficacy therapeutic strategies utilizing radiotherapy.

### SUMMARY

The present authors have studied the effects of administering CD25-ADCs in a number of different disease models, including models with CD25−ve tumor target cells. Their observations indicated an efficacy beyond what was expected for either of direct target cell killing by the ADC combined with the so-called ‘bystander effect’ indirect cell killing, as described in WO/2016/083468. Building on these observations, the present authors studies indicate that the increased efficacy of ADCx25 arises from targeted cell-killing of AD25+ve regulatory immune cells, such as Tregs. That is, the present authors have determined that the CD25-ADCs described herein have applications as powerful and specific molecular adjuvants that act to enhance and stimulate immune responses to existing or newly presented antigens.

Whilst there has been some speculation regarding the therapeutic potential of targeting of immune regulatory networks (Davies et al., Methods Mol. Biol. 1494 (2017) 107-125), as well as discussion of potential treatment strategies using, for example, metronomic chemotherapy, CD25 antibodies, CTLA4 antibodies, anti-GITR antibodies, anti-OX40, PD1 pathway modulation, Indoleamine 2,3-dioxygenase inhibitors, anti-LAG3, CCR4 antagonists, anti-FOXP3, blockade of TGFβ, Listeriolysin O, blockade of adenosine-mediated immune suppression, anti-angiogenic molecules, folate receptor 4 antibodies, nicotinamide adenine dinucleotide, TLR modulation, or ICOS antibodies (reviewed in Batista-Duharte et al., Pharmacological Research 129 (2018) 237-250), recent reports have underlined that the highly complex mechanisms that control regulatory immune cells are not yet well-defined (Berod et al., Microbiol. Biotechnol. 5 (2) (2012)260-269). For example, though some studies have shown that depletion of Tregs prior to vaccination in murine models can enhance immune responses to certain vaccines (Klages et al., Cancer Res. 70 (20) (2010) 7788-7799; Fisher et al. Immun. Inflamm. Dis. 5 (1) (2016) 16-28), other studies have identified cancer models where Treg modulation has no benefit (murine pancreatic carcinoma model—Keenan et al., Gastroenterology 146 (7) (2014) 1784-1794) or highlighted the risks of regulatory immune cell modulation such as disruption of immune tolerance, and induction of inflammatory disorders or autoimmune processes (Bayry et al., Virus disease 25 (1) (2014) 18-25; van Elsas et al., Exp. Med. 194(4) (2001) 481-489). Accordingly, the in vivo demonstration by the present authors of the molecular adjuvant activity of the CD25-ADCs described herein is unexpected.

Building upon the finding that the CD25-ADCs described herein have immune-stimulating properties, the present authors studied the effects of combining administration of the CD25-ADCs described herein with radiotherapy. These studies have demonstrated that this combination leads not only to synergistic therapeutic efficacy, but also highly advantageous therapeutic effects associated with the stimulation of an anti-tumour immune reaction such as therapeutic efficacy against neoplastic cells remote form the site of radiotherapy administration and ‘immunization’ of the subject against re-challenge with neoplastic cells derived from the original neoplasm. Furthermore, the synergistic increase in therapeutic efficacy of the anti-CD25 ADC/radiotherapy combination allows for the use of lower radiotherapy doses (and therefore reduced side-effects) than is typical for radiotherapy when used alone.

Without wishing to be bound by theory, the present authors believe the observed advantage is derived from the potential of radiotherapy to convert immunologically ‘cold’ tumors into ‘hot’ tumors by a combination of distinct mechanisms including: (a) increasing tumor immunogenicity via the upregulation of antigenic expression, antigen processing, major histocompatibility molecules, and costimulatory signals; (b) overcoming an immunosuppressive tumor microenvironment by shifting the cytokine balance in favour of immunostimulation (e.g. by increasing the production of immunostimulatory cytokines); (c) recruiting antigen-presenting and immune effector cells to the tumor microenvironment (see Ko et al., Ther Adv Med Oncol 2018, Vol. 10: 1-11, DOI: 10.1177/1758834018768240). The effect of this immunological conversion of tumours is then amplified by the immune regulatory cell depletion resulting from CD25-ADC administration. Consistent with this, relapse and regrowth of tumours following radiotherapy and PD1 blockade is associated with Treg repopulation of the tumour microenvironment (Oweida et al., Clin Cancer Res Jul. 24, 2018 DOI: 10.1158/1078-0432.CCR-18-1038).

This ‘immunological activation’ of a tumour by the administration of an anti-CD25 ADC as described herein combined with radiotherapy is believed to be further enhanced by the direct and indirect tumour-cell-killing activity of the anti-CD25 ADC. Specifically, the anti-CD25 ADCs described herein have been sown to directly kill target cells through cytotoxic ADC binding to the target cells and/or, through a ‘bystander effect’, indirectly killing target cells in the proximity of cells that are directly bound by the cytotoxic ADC (see, for example, WO/2016/083468). Without wishing to be bound by theory, it is believed that this killing of target cells causes the release of target antigens, ‘stranger signals’, ‘neo-epitopes’, and/or ‘danger signals’ into the extracellular environment where they can interact with and further stimulate a subject's immune system (see, for example, Virgil E J C Schijns & Ed C Lavelle (2011) Expert Review of Vaccines, 10:4, 539-550).

Thus, in one aspect, the present disclosure provides a method of inducing or enhancing an immune response against a disorder in a subject, the method comprising administering to the subject an effective amount of an anti-CD25 ADC in combination with radiotherapy. The immune response may be a CD8+ T cell response, a CD4+ T cell response, an antibody response, or a memory cell response.

In an aspect the present disclosure provides a method for treating a disorder in an subject, the method comprising administering to the subject an effective amount of an anti-CD25 ADC in combination with radiotherapy. In some embodiments, the method further comprises a step of selecting the subject for treatment and a subject is selected for treatment with the anti-CD25 ADC if (i) the subject has been treated with radiotherapy, (ii) the subject is being treated with radiotherapy, and/or (iii) the subject is radiosensitive.

In some cases the combination of anti-CD25 ADC administration and radiotherapy administration are steps of the method of treatment claimed herein. In other cases the claimed method of treatment comprises only the administration of the anti-CD25 ADC, with the administration of radiotherapy to give the synergistic therapeutic effect described herein falling outside of the claimed method of treatment. In those other cases the synergistic combination may be made by selecting for treatment with the anti-CD25 ADC subjects who (i) have been treated with radiotherapy, and or (ii) will be treated with radiotherapy. As noted below, preferably the anti-CD25 ADC is administered before the radiotherapy so as to reduce the immune-suppressive activity or size of a population of regulatory immune cells prior to radiotherapy.

Thus, in an aspect, the present disclosure provides a method of inducing or enhancing an immune response against a disorder in a subject, the method comprising:
(a) selecting for treatment a subject who has, is, or will be treated with radiotherapy; and
(b) administering to the subject an effective amount of an anti-CD25 ADC.

In another aspect the present invention provides a method for treating a disorder in an subject, the method comprising:
(a) selecting for treatment a subject who has, is, or will be treated with radiotherapy; and
(b) administering to the subject an effective amount of an anti-CD25 ADC.

In some cases the treatment comprises administering the anti-CD25 ADC before the radiotherapy, simultaneous with the radiotherapy, or after the radiotherapy. In some cases the immune-suppressive activity of a population of regulatory immune cells in the subject is reduced by at least 90% before the radiotherapy is administered, and/or the size of a population of regulatory immune cells in the subject is reduced by at least 90% before the radiotherapy is administered. Preferably, the regulatory immune cells are Treg cells, such as tumour-associated or tumour-infiltrating Tregs.

In some cases the subject has a disorder or has been determined to have a disorder. For example, in some cases the subject has, or has been has been determined to have, a cancer which expresses CD25 or CD25+ tumour-associated non-tumour cells, such as CD25+ infiltrating cells.

The subject is preferably human. The subject may be radiosensitive.

Preferably the radiotherapy is optimized to minimize immunosuppressive effects on immune cells and/or maximise the cytotoxic effect on the targeted tissue. In some cases the radiotherapy is sub-therapeutic dose for treatment of the disorder with radiotherapy alone.

In some cases the radiotherapy is selected from the group consisting of external beam radiotherapy, stereotactic radiation therapy, Intensity-Modulated Radiation Therapy, particle therapy, brachytherapy, delivery of radioisotopes, intraoperative radiotherapy, Auger therapy, Volumetric modulated arc therapy, Virtual simulation, 3-dimensional conformal radiation therapy, and intensity-modulated radiation therapy.

In some cases the total radiotherapy dose is no greater than 100 Gy, such as no greater than 80, 60, 40, 30, 24, 20, 16, 15, 12, or 10 Gy. In some cases the radiotherapy is delivered as a single dose. However, preferably the radiotherapy is administered as fractionated doses. In some cases, each fractionated dose is, or is no greater than, 20 Gy, 15, 12, 10, 8, 6, 5, 4, 3, or 2 Gy. In some cases, the radiotherapy is administered in two fractionated doses, or in at least two fractionated doses. In other cases the radiotherapy is administered in, or in at least, 3, 4, 5, 6, 7, 8, 9, or 10 fractionated doses. A fractionated dose may be administered once daily (QD), once every other day (Q2D), once every third day (Q3D), or once weekly (QW).

In some cases the disorder is a proliferative disease, such as cancer. The disorder may be, or characterized by one or more solid tumours. In some cases the treatment induces or enhances an immune response against a solid tumour, which tumour may be remote from the radiotherapy administration site. In some cases the solid tumour is an established tumour which may be diagnosed or identified in a naïve subject. In some cases the tumour is a relapsed tumour and/or a metastatic tumour.

The solid tumour may comprise or consist of CD25−ve neoplastic cells. In some cases the solid tumour is associated with CD25+ve infiltrating cells. The levels of CD25+ve infiltrating cells may be high. In some cases the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

In some cases the proliferative disorder or cancer is lymphoma or leukaemia, for example a proliferative disorder or cancer is selected from Hodgkin's Lymphoma, non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL), and leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disorder or cancer may be associated with elevated levels of regulatory immune cells, such as Treg cells.

In some cases the CD25-ADC is administered in combination with a checkpoint inhibitor or other immunostimulatory agent, such as a PD1 antagonist, a PD-L1 antagonist, a GITR agonist, an OX40 agonist, or a CTLA-4 antagonist. The CD25-ADC may be administered before the checkpoint inhibitor or other immunostimulatory agent, simultaneous with the checkpoint inhibitor or other immunostimulatory agent, or after the checkpoint inhibitor or other immunostimulatory agent.

In some cases the CD25-ADC is as defined herein in statements 1-110 of the section herein entitled “CD25-ADCs”, such as ADCx25. In some cases the CD25-ADC is ADCT-301 or Camidanlumab Tesirine.

In another aspect, the disclosure also provides an antibody-drug conjugate compound as disclosed herein for use in a method of treatment as disclosed herein.

In another aspect, the disclosure also provides a composition or pharmaceutical composition comprising an antibody-drug conjugate compound as disclosed herein for use in a method of treatment as disclosed herein.

In another aspect, the disclosure also provides a use of an antibody-drug conjugate compound as disclosed herein in the preparation of a medicament for use in a method of treatments as disclosed herein.

### DETAILED DESCRIPTION

The present authors have studied the effects of administering CD25-ADCs in a number of different disease models. Through these studies, the present authors have determined that the CD25-ADCs described herein have applications as powerful and specific molecular adjuvants that synergistically combine with radiotherapy to produce a number of advantageous clinical effects.

Accordingly, in one aspect the present disclosure provides a method of inducing or enhancing an immune response against a disorder in a subject, the method comprising administering to the subject an effective amount of an anti-CD25 ADC in combination with radiotherapy.

In another aspect, the present disclosure provides a method for treating a disorder in an subject, the method comprising administering to the subject an effective amount of an anti-CD25 ADC in combination with radiotherapy.

Inducing or Enhancing an Immune Response

The present disclosure provides method of inducing or enhancing an immune response in a subject.

Typically the immune response is a specific immune response directed against a disease-associated antigen (DAA). For example, in embodiments where the disorder is a proliferative disease such as a neoplasm, the immune response is preferably a cytotoxic immune response directed against the neoplastic cells.

For example, in some embodiments the specific immune response is the activation and/or proliferation of CD8+ve T-cells (commonly referred to as T-Helper cells, or Tₕ cells). In some embodiments the specific immune response is the activation and/or proliferation of anti-tumour CD4+ve T-cells (commonly referred to as cytotoxic T-cells, or T_{c} cells). In some embodiments the specific immune response is the activation and/or proliferation of B-cells and the associated upregulation of anti-tumour antibodies.

In some embodiments the specific immune response is the generation of memory cells. The memory cells may be memory T-cells or memory B-cells. The memory T-cells may be memory Tₕ-cells or memory T_{c}-cells.

The term ‘inducing or enhancing an immune response’ as used herein refers to creating, or increasing the level of, the relevant immune response through administration of the CD25-ADC. The enhancement may be relative to, for example, a control subject or population of subjects who have not received the CD25-ADC.

In some embodiments the existence or level of the immune response may be assessed by measuring the titer of a specific cell or molecule in a sample taken from a subject. For example, the existence or level of a DAA-specific T_{c}-cell response can be assessed by identifying and counting the relevant cells in a sample of peripheral blood taken from a subject.

In some embodiments, induction of an immune response means that following administration of the CD25-ADC the level of immune response is increased from an undetectable to a detectable level. In some embodiments an immune response is deemed to have ben induced if administration of the CD25-ADC is effective in preventing, ameliorating and/or treating a disorder such as a proliferative disorder. Prevention encompasses inhibiting or reducing the spread of the disorder or inhibiting or reducing the onset, development or progression of one or more of the symptoms associated with the disorder. Amelioration as used in herein may refer to the reduction of visible or perceptible disorder symptoms, or any other measurable manifestation of the disorder.

In some embodiments, enhancement of an immune response means that following administration of the CD25-ADC the level of the immune response is increased by at least 10%, such as by at least 20%, at least 30%, at least 40%, at least 40%, at least 50%, at least 60%, at least 70%, at least 80%, at least 90%, at least 100%, at least 150%, at least 200%, at least 300%, at least 400%, or at least 500%.

In some embodiments the level of immune response is assessed by determining the number of activated CD4+ve T-cell cells. In some embodiments the level of immune response is assessed by determining the size of certain cell populations, such as NK cells, monocyes, or dendritic cells). In some embodiments the level of immune response is assessed by determining the titre of a specific antibody or antibodies, for example the titre of an anti-tumour antibody or antibodies.

In some embodiments the immune response is induced or enhanced by administering the CD25-ADC to the subject in combination with a second immunostimulatory agent. For example, the CD25-ADC may be administered to the subject in combination with a CD3/DAA bispecific T-cell engager (BiTE), an anti-CD47 therapeutic, a PD-1 inhibitor, a PDL-1 inhibitor, a GITR agonist, an OX40 agonist, or a CTLA-1 antagonist, In some embodiments the second immunostimulatory agent used as a monotherapy does not induce a significant immune response in the subject.

Treating Disorders by Inducing or Enhancing an Immune Response

The present disclosure provides a method of treating or preventing a disorder a subject by inducing or enhancing an immune response.

Disorders that can be treated by inducing or enhancing an immune response include proliferative diseases such as cancer, wherein neoplastic cells express one or more antigen that is either (i) not expressed on non-neoplastic cells, or more usually (ii) expressed at ha higher level on neoplastic cells than on non-neoplastic cells. Examples of this type of disorder include most prostate cancer (key antigen=PSMA) and some breast cancers (key antigen=HER2).

Without wishing to be bound by theory, the efficacy of the treatment methods described herein is believed to arise from the induction and/or enhancement of the subject's immune response against antigen(s) characteristic of the disorder. This induction and/or enhancement is believed to be due to a combination of:
(1) the administration of the CD25-ADC reducing the immune-suppressive activity of a population of immune regulatory cells (such as CD25+ve T_{reg} cells);
(2) the release of disorder-associated antigens and immune-stimulating molecules through the targeted and localised cell-killing activity of radiotherapy.

The present authors have demonstrated that this combined effect is particularly potent in the treatment of solid tumours, where the tumour itself can be readily targeted with radiotherapy. In addition, a number of proliferative disorders have been reported to be associated with an increased number of immune regulatory cells such as T_{reg} cells. Without wishing to be bound by theory, it is believed that for these tumours reducing the immune-suppressive activity of immune regulatory cells by administration of the anti-CD25 ADCs described herein will have a pronounced effect on the ability of the subject's immune system to recognise and kill tumour cells.

Treg Cells

Tregs are identified by surface expression of CD4+ve/CD25^{high}/CD127^{low/-} [33] and form two main subsets: natural Tregs (nTregs), which are thymus-derived, and induced, adaptive or peripheral Tregs (pTregs), which are derived from naive CD4+ T cells under a variety of conditions (Curotto de Lafaille et al., Immunity 30 (2009)626-635-).

Tregs have immunosuppressive activity, with the more important effector T cells (Teffs) that are suppressed by Tregs being Th1 (control of infections and tumors), Th2 (effectors against extra-cellular parasites, including helminths), Th17 (play an important role in pathogen clearance at mucosal surfaces, are effective against pathogenic fungi and are also involved in different inflammatory process) and CTLs (cytotoxic T cells). Treg-mediated suppression can be accomplished in two ways: by cell-cell contact or by means of soluble mediators and cytokines (paracrine signaling). Several mechanisms have been identified as involved in the immunosuppressive function of Tregs, including:
(1) inhibition by immunoregulatory cytokines such as TGFβ, IL-10, and IL-35;
(2) cytolysis of effector cells by producing granzyme and perforin;
(3) metabolic interruption, including inhibition of proliferative response via the IL-2 receptor, cAMP-mediated metabolic inhibition, tryptophane depletion and immunomodulation mediated by the A2 adenosine receptor or kynurenine; and
(4) interactions with dendritic cells that modulates their function and maturation (Batista-Duharte et al. 2018, ibid.; Arce-Sillas, et al., J. Immunol. Res. 2016, 1720827).

Sequence of Administration

As described elsewhere herein, and without wishing to be bound by theory, it is believed the synergistic therapeutic effect observed with the combination of CD25-ADC and radiotherapy treatment arises at least in part from immunogenic tumour cell death caused by radiotherapy happening during the period when the activity or size of a population of immune regulatory cells—such as CD25+ve regulatory T-cells—has been reduced by administration of the anti-CD25-ADC. Accordingly, in order to maximise this effect the CD25-ADC is preferably administered sufficiently before the radiotherapy in order to allow the ADC time to bring about a clinically relevant reduction of the regulatory immune cell population. The examples herein also indicate that the population of immune regulatory cells depleted by CD25-ADC administration recovers over time (see, for example, FIGS. 10 to 16). Accordingly, the radiotherapy is preferably administered to the subject before the population of regulatory immune cells reduced by the CD25-ADC significantly recovers.

Preferably, the CD25-ADC is administered before the radiotherapy; for example, sufficiently before the radiotherapy such that the size or activity of a regulatory immune cell population (such as CD25+ve Treg cells) is significantly reduced. For example, the CD25-ADC may be administered 1 hour, 2 hours, 6 hours, 12 hours, or 24 hours before the radiotherapy. The CD25-ADC may be administered 1 day, 2 days, 3 days, 4 days, 5 days, 6 days, or 7 days before the radiotherapy. In other embodiments, the CD25-ADC may be administered at least 1 hour, 2 hours, 6 hours, 12 hours, or 24 hours before the radiotherapy. The CD25-ADC may be administered at least 1 day, 2 days, 3 days, 4 days, 5 days, 6 days, or 7 days before the radiotherapy. Preferably the CD25-ADC is administered at least 1 day before the radiotherapy, even more preferably at least 2 days before the radiotherapy.

Preferably, the CD25-ADC is administered no longer before the radiotherapy than the time required for the size or activity of the regulatory immune cell population (such as CD25+ve Treg cells) reduced by CD25-ADC administration to substantially recover its pre-ADC treatment level. For example, the CD25-ADC may be administered no longer than 3 months before the radiotherapy, such as no longer than 2 months, or 1 month before the radiotherapy. In some cases the CD25-ADC may be administered no longer than 28 days before the radiotherapy, such as no longer than 21 days, 18 days, 14 days, 11 days, 7 days, 6 days, 5 days, 4 days, 3 days, or 2 days before the radiotherapy. Preferably the CD25-ADC is administered no longer than 21 days before the radiotherapy.

Thus, in some cases the CD25-AC is administered 1 hour to 3 months before the radiotherapy, such as 1 to 21 days before the radiotherapy, or 2 to 14 days before the radiotherapy.

Although administration of the CD25-ADC before the radiotherapy is preferred, it is contemplated that a synergistic therapeutic effect can also be observed under some circumstances by concommitant administration of the ADC25-ADC, or administration of the radiotherapy before the CD25-ADC. This is because the immunogenically activated state of a tumour that is induced by radiotherapy is expected to persist for a period of time, with CD25-ADC-induced reduction of the size or activity of a regulatory immune cell population during this period expected to lead to the synergistic therapeutic effect reported herein.

Accordingly, in some cases the CD25-ADC and radiotherapy are administered concomitantly (for example, on the same day). In some cases the radiotherapy is administered before the CD25-ADC; for example, no longer before the CD25-ADC that the time required for the tumour treated with radiotherapy to substantially recover from the immunologically activated state induced by the radiotherapy. In some cases the radiotherapy is administered no longer than 4 weeks before the CD25-ADC, such as no longer than 3 weeks, 2 weeks, 1 week, 6 days, 5 days, 4 days, 3 days, 2 days, or 1 day before the CD25-ADC.

In some embodiments the immune-suppressive activity of a population of immune regulatory cells is reduced before the radiotherapy is administered to the subject. In some embodiments the reduction in the immune-suppressive activity of the immune regulatory cell population is achieved by killing a proportion of the population. In some embodiments the reduction in the immune-suppressive activity of the immune regulatory cell population is achieved by inhibiting the immune-suppressive activity of a proportion of the regulatory cell population without killing the cells.

The immune regulatory cells may be myeloid-derived suppressor cells (MDSCs), mesenchymal stromal cells (MSCs), Type II NKT cells, Treg cells, or any other immune regulatory immune cells as defined herein. Preferably, the reduced population is a population of Tregs, such as tumour-associated or tumour-infiltrating Tregs.

Preferably the immune regulatory cells whose immune-suppressive activity is reduced are Treg cells. The term “Treg” cells as used herein refers to regulatory T-cells. This cell population may be identified by the following pattern of surface-marker expression: CD4+ve, CD25^{high}, CD127^{low/-} (see Yu et al. 2012, Inflammation 35(6), pp. 1773-1780).

In some embodiments the immune-suppressive activity of a population of immune regulatory cells is reduced by at least 10%, at least 20%, at least 30%, at least 40%, at least 50%, at least 60%, at least 70%, at least 70%, at least 90%, at least 95%, or at least 98% before the radiotherapy is administered to the subject. Preferably, the reduction is measured relative to levels in the same subject prior to the administration of the CD25-ADC.

In some embodiments the immune-suppressive activity of a population of immune regulatory cells is assessed by measuring the level of inhibitory cytokines such as IL-10, TGFβ, or IL-35 (see Bettini et al., Current opinion in immunology. 2009; 21(6):612-618). In some embodiments the immune-suppressive activity of a population of immune regulatory cells is assessed by measuring the expression of specific genes such as LAYN, MADEH1, or CCR8 (see de Simone et al., Immunity. 2016 Nov. 15; 45(5): 1135-1147).

In some embodiments the size of a population of immune regulatory cells is reduced by at least 10%, at least 20%, at least 30%, at least 40%, at least 50%, at least 60%, at least 70%, at least 70%, at least 90%, at least 95%, or at least 98% before the radiotherapy is administered to the subject. Preferably, the reduction in population size is measured relative to levels in the same subject prior to the administration of the CD25-ADC.

In some embodiments the population of immune regulatory cells is measured systemically, for example by using FACS on a representative sample such as whole blood, bone-marrow, lymph-nodes, spleen, peyer-plaques, or tonsils. In some embodiments the population of immune regulatory cells is measured locally, for example in a sample taken from a tumour or the tumour microenvironment. The local population of immune regulatory cells may be measured by, for example, FACS, immunohistochemistry or immunofluorescence of tissue sections. Alternatively, techniques such as RNAscope® may be used on tissue sections to quantify immune regulatory cells in biopsies. Local measurement of cell population is preferred in situations where local measurement is possible (for example, for solid tumours).

In some embodiments the CD25-ADC is administered concomitantly with the radiotherapy.

The radiotherapy may be administered to the subject before the CD25-ADC, concomitantly with the CD25-ADC, or after the CD25-ADC

CD25 ADCs

As used herein, the term “CD25-ADC” refers to an ADC in which the antibody component is an anti-CD25 antibody. Preferably the CD25-ADC comprises a pyrrolobenzodiazepine (PBD) drug moiety, optionally wherein the PBD drug moiety is linked to the antibody by a cleavable linker.

In a preferred aspect, the CD25-ADC has the structure defined in the following paragraphs:

1. A conjugate of formula L-(D^{L})ₚ, where D^{L} is of formula I or II:

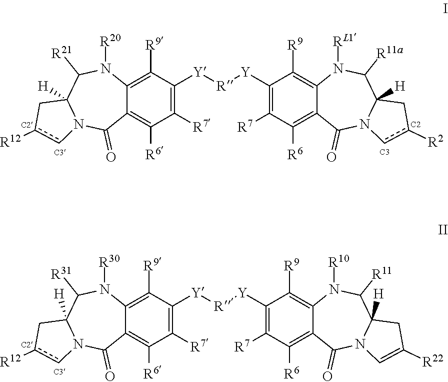

wherein:
L is an antibody (Ab) which is an antibody that binds to CD25;
   when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

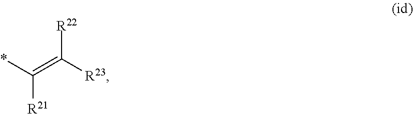

wherein each of R²¹, R²² and R²³ are independently selected from H, 1-3 saturated alkyl, C₂₋₃ alkenyl, C₂₋₃ alkynyl and cyclopropyl, where the total number of carbon atoms in the R¹² group is no more than 5;

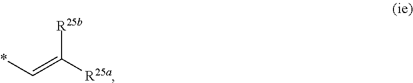

wherein one of R^{25a} and R^{25b} is H and the other is selected from: phenyl, which phenyl is optionally substituted by a group selected from halo, methyl, methoxy; pyridyl; and thiophenyl; and

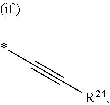

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is

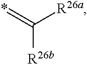

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
[Formula I]
R^{L1}′ is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

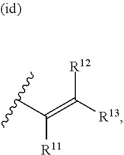

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

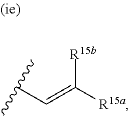

wherein one of R^{15a} and R^{15b} is H and the other is selected from: phenyl, which phenyl is optionally substituted by a group selected from halo, methyl, methoxy; pyridyl; and thiophenyl; and

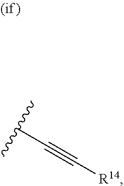

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is

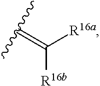

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[Formula II]
R²² is of formula IIIa, formula IIIb or formula IIIc:

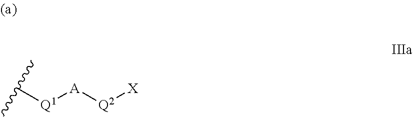

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

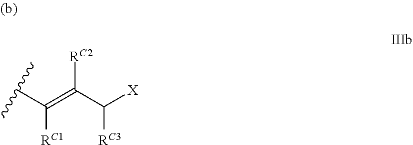

where;
R^{C1}, R^{C2} and R^{C3} are independently selected from H and unsubstituted C₁₋₂ alkyl;

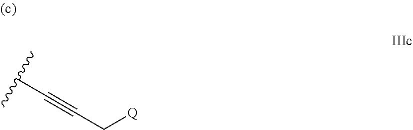

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′},
NHNH—R^{L2′}, CONHNH—R^{L2′},

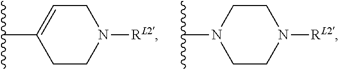

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.
2. The conjugate according to statement 1, wherein the conjugate is not:
ConjA

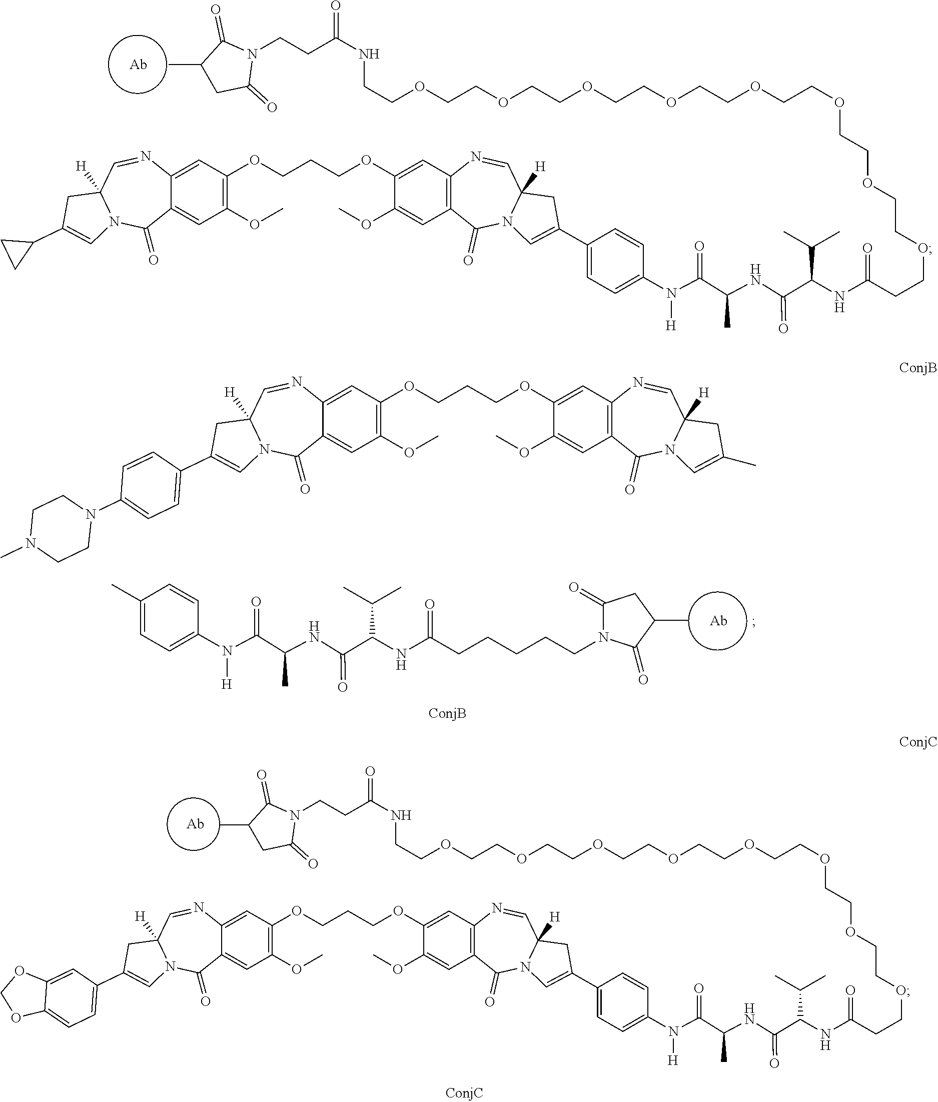

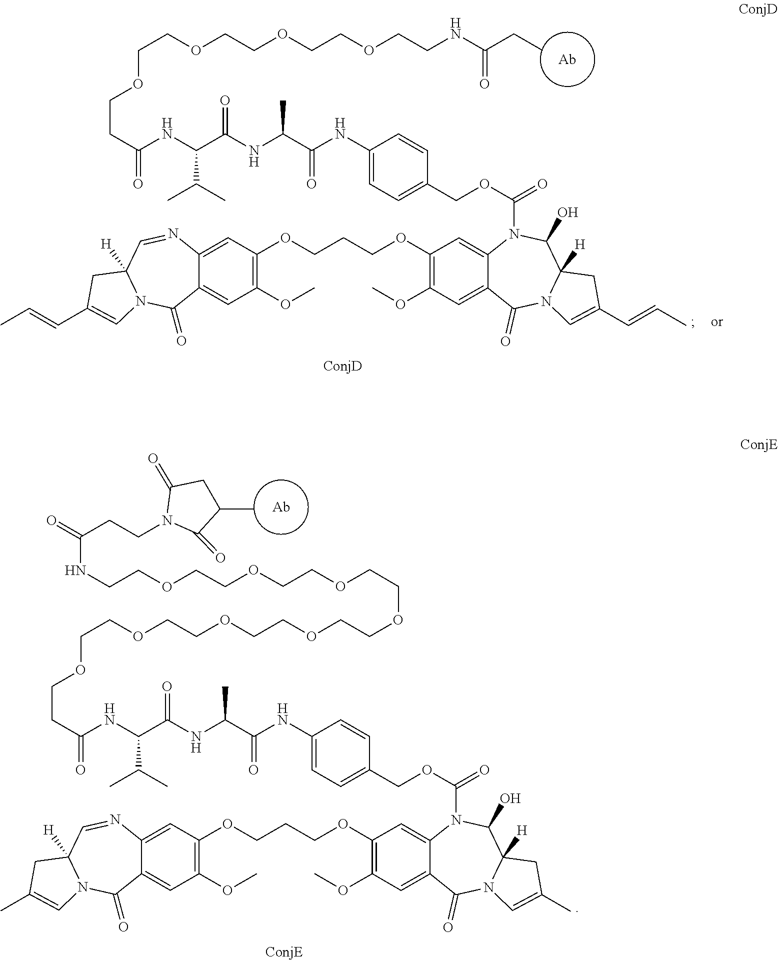

3. The conjugate according to either statement 1 or statement 2, wherein R⁷ is selected from H, OH and OR.
4. The conjugate according to statement 3, wherein R⁷ is a C₁₋₄ alkyloxy group.
5. The conjugate according to any one of statements 1 to 4, wherein Y is O.
6. The conjugate according to any one of the preceding statements, wherein R″ is C₃₋₇ alkylene.
7. The conjugate according to any one of statements 1 to 6, wherein R⁹ is H.
8. The conjugate according to any one of statements 1 to 7, wherein R⁶ is selected from H and halo.
9. The conjugate according to any one of statements 1 to 8, wherein there is a double bond between C2′ and C3′, and R¹² is a C₅₋₇ aryl group.
10. The conjugate according to statement 9, wherein R¹² is phenyl.
11. The conjugate according to any one of statements 1 to 8, wherein there is a double bond between C2′ and C3′, and R¹² is a C₈₋₁₀ aryl group.
12. The conjugate according to any one of statements 9 to 11, wherein R¹² bears one to three substituent groups.
13. The conjugate according to any one of statements 9 to 12, wherein the substituents are selected from methoxy, ethoxy, fluoro, chloro, cyano, bis-oxy-methylene, methyl-piperazinyl, morpholino and methyl-thiophenyl.
14. The conjugate according to any one of statements 1 to 8, wherein there is a double bond between C2′ and C3′, and R¹² is a C₁₋₅ saturated aliphatic alkyl group.
15. A compound according to statement 16, wherein R¹² is methyl, ethyl or propyl.
16. The conjugate according to any one of statements 1 to 8, wherein there is a double bond between C2′ and C3′, and R¹² is a C₃₋₆ saturated cycloalkyl group.
17. The conjugate according to statement 16, wherein R¹² is cyclopropyl.
18. The conjugate according to any one of statements 1 to 8, wherein there is a double bond between C2′ and C3′, and R¹² is a group of formula:

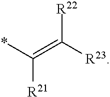

19. The conjugate according to statement 18, wherein the total number of carbon atoms in the R¹² group is no more than 4.
20. The conjugate according to statement 19, wherein the total number of carbon atoms in the R¹² group is no more than 3.
21. The conjugate according to any one of statements 18 to 20, wherein one of R²¹, R²² and R²³ is H, with the other two groups being selected from H, C₁₋₃ saturated alkyl, C₂₋₃ alkenyl, C₂₋₃ alkynyl and cyclopropyl.
22. The conjugate according to any one of statements 18 to 20, wherein two of R²¹, R²² and R²³ are H, with the other group being selected from H, C₁₋₃ saturated alkyl, C₂₋₃ alkenyl, C₂₋₃ alkynyl and cyclopropyl.
23. The conjugate according to any one of statements 1 to 8, wherein there is a double bond between C2′ and C3′, and R¹² is a group of formula:

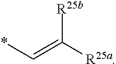

24. The conjugate according to statement 23, wherein R¹² is the group:

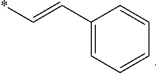 [SMILES: */C=C/c1ccccc1]

25. The conjugate according to any one of statements 1 to 8, wherein there is a double bond between C2′ and C3′, and R¹² is a group of formula:

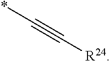

26. The conjugate according to statement 25, wherein R²⁴ is selected from H, methyl, ethyl, ethenyl and ethynyl.
27. The conjugate according to statement 26, wherein R²⁴ is selected from H and methyl.
28. The conjugate according to any one of statements 1 to 8, wherein there is a single bond between C2′ and C3′, R¹² is

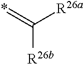

and R^{26a} and R^{26b} are both H.
29. The conjugate according to any one of statements 1 to 8, wherein there is a single bond between C2′ and C3′, R¹² is

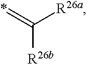

and R^{26a} and R^{26b} are both methyl.
30. The conjugate according to any one of statements 1 to 8, wherein there is a single bond between C2′ and C3′, R¹² is

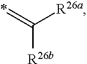

one of R^{26a} and R^{26b} is H, and the other is selected from C₁₋₄ saturated alkyl, C₂₋₃ alkenyl, which alkyl and alkenyl groups are optionally substituted.
[Formula I]
31. The conjugate according to any one of statements 1 to 30, wherein there is a double bond between C2 and C3, and R² is a C₅₋₇ aryl group.
32. The conjugate according to statement 31, wherein R² is phenyl.
33. The conjugate according to any one of statements 1 to 30, wherein there is a double bond between C2 and C3, and R¹ is a C₈₋₁₀ aryl group.
34. A compound according to any one of statements 31 to 33, wherein R² bears one to three substituent groups.
35. The conjugate according to any one of statements 31 to 34, wherein the substituents are selected from methoxy, ethoxy, fluoro, chloro, cyano, bis-oxy-methylene, methyl-piperazinyl, morpholino and methyl-thiophenyl.
36. The conjugate according to any one of statements 1 to 30, wherein there is a double bond between C2 and C3, and R² is a C₁₋₅ saturated aliphatic alkyl group.
37. The conjugate according to statement 36, wherein R² is methyl, ethyl or propyl.
38. The conjugate according to any one of statements 1 to 30, wherein there is a double bond between C2 and C3, and R² is a C₃₋₆ saturated cycloalkyl group.
39. The conjugate according to statement 38, wherein R² is cyclopropyl.
40. The conjugate according to any one of statements 1 to 30, wherein there is a double bond between C2 and C3, and R² is a group of formula:

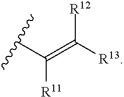

41. The conjugate according to statement 40, wherein the total number of carbon atoms in the R² group is no more than 4.
42. The conjugate according to statement 41, wherein the total number of carbon atoms in the R² group is no more than 3.
43. The conjugate according to any one of statements 40 to 42, wherein one of R¹¹, R¹² and R¹³ is H, with the other two groups being selected from H, C₁₋₃ saturated alkyl, C₂₋₃ alkenyl, C₂₋₃ alkynyl and cyclopropyl.
44. The conjugate according to any one of statements 40 to 42, wherein two of R¹¹, R¹² and R¹³ are H, with the other group being selected from H, C₁₋₃ saturated alkyl, C₂₋₃ alkenyl, C₂₋₃ alkynyl and cyclopropyl.
45. The conjugate according to any one of statements 1 to 30, wherein there is a double bond between C2 and C3, and R² is a group of formula:

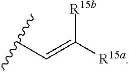

46. The conjugate according to statement 45, wherein R² is the group:

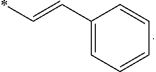 [SMILES: */C=C/c1ccccc1]

47. The conjugate according to any one of statements 1 to 30, wherein there is a double bond between C2 and C3, and R² is a group of formula:

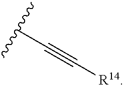

48. The conjugate according to statement 48, wherein R¹⁴ is selected from H, methyl, ethyl, ethenyl and ethynyl.
49. The conjugate according to statement 48, wherein R¹⁴ is selected from H and methyl.
50. The conjugate according to any one of statements 1 to 30, wherein there is a single bond between C2 and C3, R² is

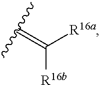

and R¹⁶ and R^{16b} are both H.
51. The conjugate according to any one of statements 1 to 30, wherein there is a single bond between C2 and C3, R² is

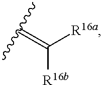

and R^{16a} and R^{16b} are both methyl.
52. The conjugate according to any one of statements 1 to 30, wherein there is a single bond between C2 and C3, R² is

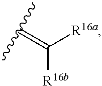

one of R^{16a} and R^{16b} is H, and the other is selected from C₁₋₄ saturated alkyl, C₂₋₃ alkenyl, which alkyl and alkenyl groups are optionally substituted.
53. The conjugate according to any one of statements 1 to 52, wherein R^{11a} is OH.
54. The conjugate according to any one of statements 1 to 53, wherein R²¹ is OH.
55. The conjugate according to any one of statements 1 to 53, wherein R²¹ is OMe.
56. The conjugate according to any one of statements 1 to 55, wherein R²⁰ is H.
57. The conjugate according to any one of statements 1 to 55, wherein R²⁰ is R^{C}.
58. The conjugate according to statement 57, wherein R^{C} is selected from the group consisting of: Alloc, Fmoc, Boc, Troc, Teoc, Psec, Cbz and PNZ.
60. The conjugate according to statement 57, wherein R^{C} is a group:

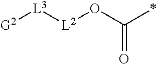

where the asterisk indicates the point of attachment to the N10 position, G² is a terminating group, L³ is a covalent bond or a cleavable linker L¹, L² is a covalent bond or together with OC(═O) forms a self-immolative linker.
61. The conjugate according to statement 60, wherein G² is Ac or Moc or is selected from the group consisting of: Alloc, Fmoc, Boc, Troc, Teoc, Psec, Cbz and PNZ.
62. The conjugate according to any one of statements 1 to 53, wherein R²⁰ and R²¹ together form a double bond between the nitrogen and carbon atoms to which they are bound.
[Formula II]
63. The conjugate according to any one of statements 1 to 30, wherein R²² is of formula IIIa, and A is phenyl.
64. The conjugate according to any one of statements 1 to 30 and statement 63, wherein R²² is of formula IIa, and Q¹ is a single bond.
65. The conjugate according to statement 63, wherein Q² is a single bond.
66. The conjugate according to statement 63, wherein Q² is —Z—(CH₂)ₙ—, Z is O or S and n is 1 or 2.
67. The conjugate according any one of statements 1 to 30 and statement 63, wherein R²² is of formula IIIa, and Q¹ is —CH═CH—.
68. The conjugate according to any one of statements 1 to 30, wherein R²² is of formula IIIb, and R^{C1}, R^{C2} and R^{C3} are independently selected from H and methyl.
69. The conjugate according to statement 68, wherein R^{C1}, R^{C2} and R^{C3} are all H.
70. The conjugate according to statement 68, wherein R^{C1}, R^{C2} and R^{C3} are all methyl.
71. The conjugate according to any one of statements 1 to 30 and statements 63 to 70, wherein R²² is of formula IIIa or formula IIIb and X is selected from O—R^{L2′}, S—R^{L2′}, CO₂—R^{L2′}, —N—C(═O)—R^{L2′} and NH—R^{L2′}.
72. The conjugate according to statement 71, wherein X is NH—R^{L2′}.
73. The conjugate according to any one of statements 1 to 30, wherein R²² is of formula IIIc, and Q is NR^{N}—R^{L2′}.
74. The conjugate according to statement 73, wherein R^{N} is H or methyl.
75. The conjugate according to any one of statements 1 to 30, wherein R²² is of formula IIIc, and Q is O—R^{L2′} or S—R^{L2′}.
76. The conjugate according to any one of statements 1 to 30 and statements 63 to 75, wherein R¹¹ is OH.
77. The conjugate according to any one of statements 1 to 30 and statements 63 to 75, wherein R¹¹ is OMe.
78. The conjugate according to any one of statements 1 to 30 and statements 63 to 77, wherein R¹⁰ is H.
79. The conjugate according to any one of statements 1 to 30 and statements 63 to 75, wherein R¹⁰ and R¹¹ together form a double bond between the nitrogen and carbon atoms to which they are bound.
80. The conjugate according to any one of statements 1 to 30 and statements 63 to 79, wherein R³¹ is OH.
81. The conjugate according to any one of statements 1 to 30 and statements 63 to 79, wherein R³¹ is OMe.
82. The conjugate according to any one of statements 1 to 30 and statements 63 to 81, wherein R³⁰ is H.
83. The conjugate according to any one of statements 1 to 30 and statements 63 to 79, wherein R³⁰ and R³¹ together form a double bond between the nitrogen and carbon atoms to which they are bound.
84. The conjugate according to any one of statements 1 to 83, wherein R^{6′}, R^{7′}, R^{9′}, and Y′ are the same as R⁶, R⁷, R⁹, and Y.
85. The conjugate according to any one of statements 1 to 84 wherein, wherein L-R^{L1′} or L-R^{L2′} is a group:

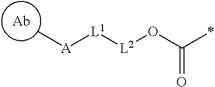

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.
86. The conjugate of statement 85, wherein L¹ is enzyme cleavable.
87. The conjugate of statement 85 or statement 86, wherein L¹ comprises a contiguous sequence of amino acids.
88. The conjugate of statement 87, wherein L¹ comprises a dipeptide and the group —X₁—X₂— in dipeptide, —NH—X₁-X₂—CO—, is selected from:
   -Phe-Lys-,
   -Val-Ala-,
   -Val-Lys-,
   -Ala-Lys-,
   -Val-Cit-,
   -Phe-Cit-,
   -Leu-Cit-,
   -Ile-Cit-,
   -Phe-Arg-,
   -Trp-Cit-.
89. The conjugate according to statement 88, wherein the group —X₁—X₂— in dipeptide, —NH—X₁-X₂—CO—, is selected from:
   -Phe-Lys-,
   -Val-Ala-,
   -Val-Lys-,
   -Ala-Lys-,
   -Val-Cit-.
90. The conjugate according to statement 89, wherein the group —X₁—X₂— in dipeptide, —NH—X₁-X₂—CO—, is -Phe-Lys-, -Val-Ala- or -Val-Cit-.
91. The conjugate according to any one of statements 88 to 90, wherein the group X₂—CO— is connected to L².
92. The conjugate according to any one of statements 88 to 91, wherein the group NH—X₁— is connected to A.
93. The conjugate according to any one of statements 88 to 92, wherein L² together with OC(═O) forms a self-immolative linker.
94. The conjugate according to statement 93, wherein C(═O)O and L² together form the group:

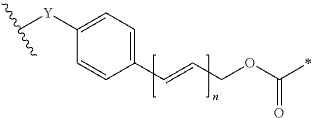

where the asterisk indicates the point of attachment to the PBD, the wavy line indicates the point of attachment to the linker L¹, Y is NH, O, C(═O)NH or C(═O)O, and n is 0 to 3.
95. The conjugate according to statement 94, wherein Y is NH.
96. The conjugate according to statement 94 or statement 95, wherein n is 0.
97. The conjugate according to statement 95, wherein L¹ and L² together with —OC(═O)— comprise a group selected from:

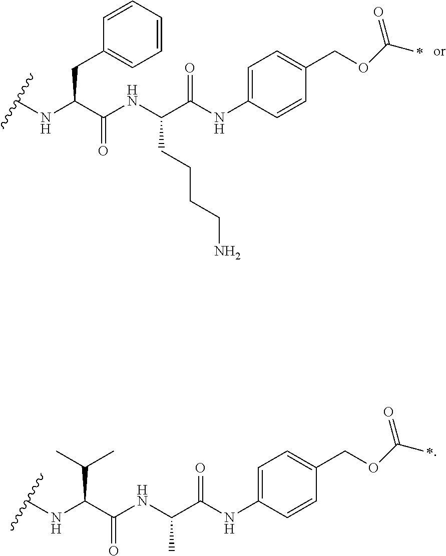

where the asterisk indicates the point of attachment to the PBD, and the wavy line indicates the point of attachment to the remaining portion of the linker L¹ or the point of attachment to A.
98. The conjugate according to statement 97, wherein the wavy line indicates the point of attachment to A.
99. The conjugate according to any one of statements 85 to 98, wherein A is:

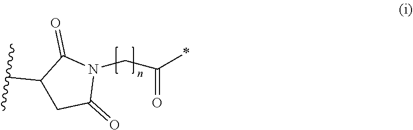

where the asterisk indicates the point of attachment to L¹, the wavy line indicates the point of attachment to the antibody, and n is 0 to 6; or

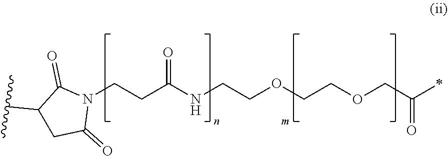

where the asterisk indicates the point of attachment to L¹, the wavy line indicates the point of attachment to the antibody, n is 0 or 1, and m is 0 to 30.
100. A conjugate according to statement 1 of formula ConjA:

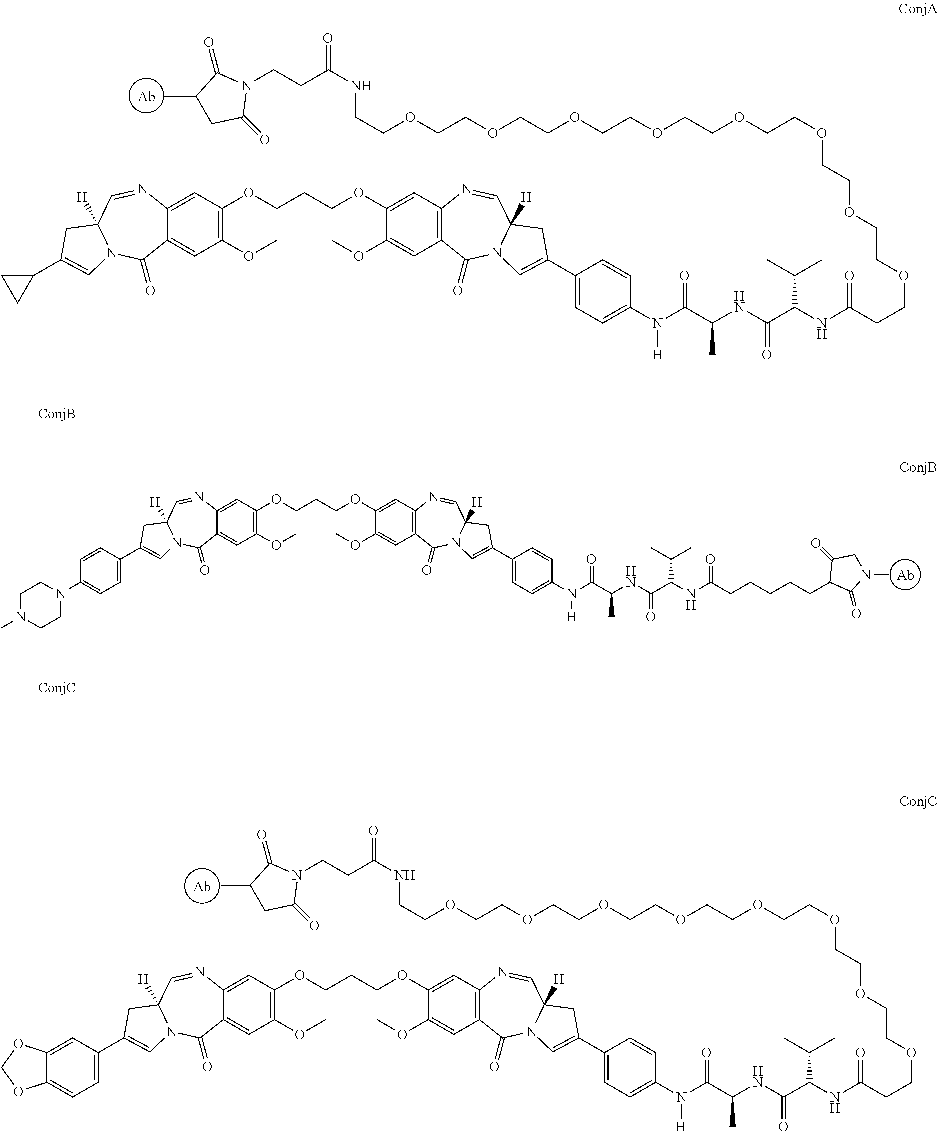

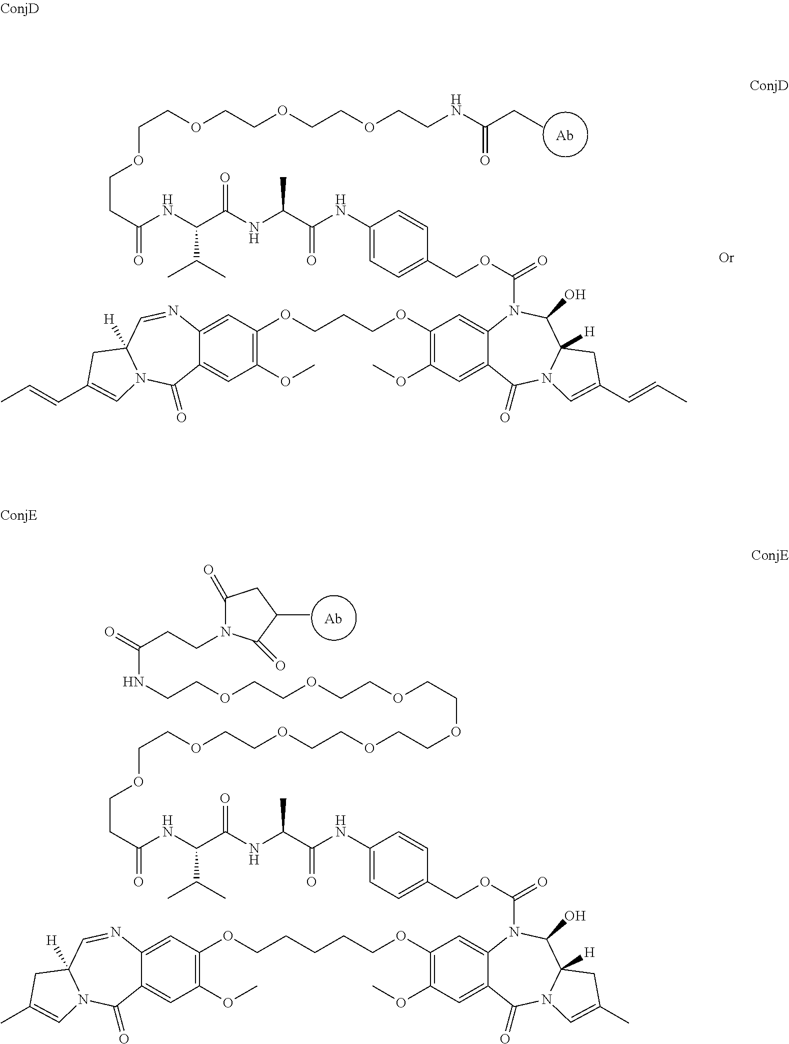

101. The conjugate according to any one of statements 1 to 100 wherein the antibody comprises:
   a VH domain having a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5.
102. The conjugate according to any one of statements 1 to 101 wherein the antibody comprises a VH domain having the sequence according to SEQ ID NO. 1.
103. The conjugate according to any one of statements 1 to 102 wherein the antibody comprises:
   a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8.
104. The conjugate according to any one of statements 1 to 103 wherein the antibody comprises a VL domain having the sequence according to SEQ ID NO. 2.
105. The conjugate according to any one of statements 1 to 103 wherein the antibody in an intact antibody.
106. The conjugate according to any one of statements 1 to 105 wherein the antibody is humanised, deimmunised or resurfaced.
107. The conjugate according to any one of statements 1 to 104 wherein the antibody is a fully human monoclonal IgG1 antibody, preferably IgG1,κ.
108. The conjugate according to any one of statements 1 to 107 wherein the drug loading (p) of drugs (D) to antibody (Ab) is an integer from 1 to about 8.
109. The conjugate according to statement 108, wherein p is 1, 2, 3, or 4.
110. The conjugate according to statement 108 comprising a mixture of the antibody-drug conjugate compounds, wherein the average drug loading per antibody in the mixture of antibody-drug conjugate compounds is about 2 to about 5.

The definition of the terms used in the above statement are as defined in WO2014/057119.

### Preferred CD25-ADC Embodiments

The term anti-CD25-ADC may include any embodiment described in WO 2014/057119. In particular, in preferred embodiments the ADC has the chemical structure:

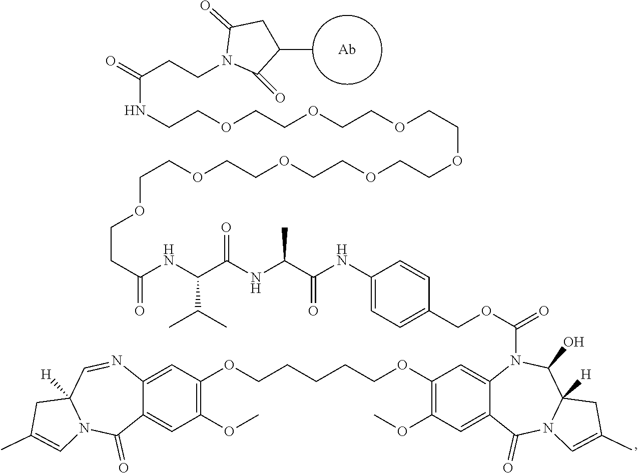

where the Ab is a CD25 antibody, and the DAR is between 1 and 8.

The antibody may comprise a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5.

In some aspects the antibody component of the anti-CD25-ADC is an antibody comprising: a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5. In some embodiments the antibody comprises a VH domain having the sequence according to SEQ ID NO. 1.

The antibody may further comprise: a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In preferred embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,κ.

In some embodiments the antibody is the AB12 antibody described in WO 2004/045512 (Genmab A/S).

In an aspect the antibody is an antibody as described herein which has been modified (or further modified) as described below. In some embodiments the antibody is a humanised, deimmunised or resurfaced version of an antibody disclosed herein.

The most preferred anti-CD25-ADCs for use with the aspects of the present disclosure is ADCX25/ADCT-301/Camidanlumab Tesirine; the structure of ADCx25 is described herein below.

ADCx25

ADCx25 is an antibody drug conjugate composed of a human antibody against human CD25 attached to a pyrrolobenzodiazepine (PBD) warhead via a cleavable linker. The mechanism of action of ADCX25 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

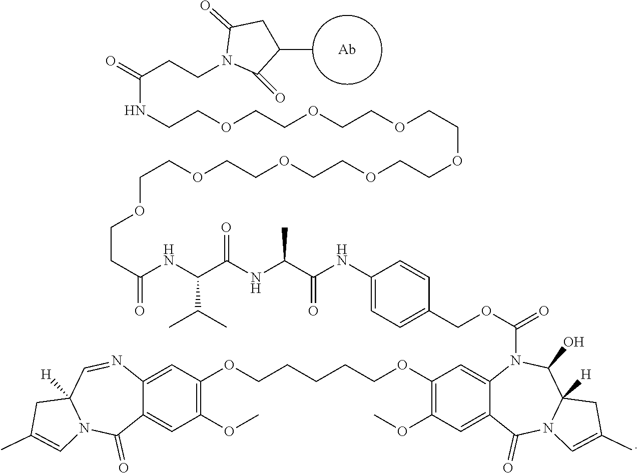

Ab represents Antibody AB12 (fully human monoclonal IgG1, K antibody with the VH and VL sequences SEQ ID NO. 1 and SEQ ID NO. 2, respectively, also known as HuMax-TAC). It is synthesised as described in WO 2014/057119 (Conj AB12-E) and typically has a DAR (Drug to Antibody Ratio) of 2.0+/−0.3.

CD25 Binding

The “first target protein” (FTP) as used herein is preferably CD25.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

Radiotherapy

In an aspect the CD25-ADCs described herein are administered in combination with radiotherapy.

As used herein, the terms “radiation therapy” or “radiotherapy” may refer to the medical use of ionizing radiation as part of cancer treatment to control or eradicate malignant cells. Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy.

In one embodiment, radiatiotherapy uses high-energy radiation to shrink tumors and kill cancer cells. The radiation may be, for example, X-rays, gamma rays, or charged particles. Modes of cell killing through radiation include DNA damage either directly or by creating free radicals within cells that in turn damage DNA.

Radiation may be delivered by a machine outside the body (external-beam radiation therapy), or may come from radioactive material placed in the body near cancer cells (internal radiation therapy, also called brachy therapy). In one example of systemic radiation therapy, radioactive substances, such as radioactive iodine, are used which travel in the blood to kill cancer cells.

Preferably, the radiotherapy is administered in a regime designed to minimize any immunosuppressive effects of the radiation. For example, preclinical evidence indicates high radiation doses above 12-18 Gy result in an attenuation of tumor immunogenicity (Vanpouille-Box C., et al., Nat Commun 2017; 8: 15618). In addition, it is known that circulating lymphocytes are particularly radiosensitive (see Yovino S., et al., Cancer Invest 2013; 31: 140-144); this indicates radiotherapy regimes aimed at stimulating an anti-tumour immune response should aim to minimise both (1) the amount of vasculature exposed in each treatment, and (2) the number of exposures in the treatment regime.

Accordingly, preferably the dose and administration pattern of radiotherapy is optimized to minimize immunosuppressive effects on immune cells whilst maximizing the immunogenic cell-death effects on tumour cells.

Furthermore, the synergistic increase in therapeutic efficacy of the anti-CD25 ADC and radiotherapy combination allows for the use of a lower total dose of radiotherapy, thereby reducing side-effects arising from the irradiation of healthy tissues. In some embodiments, the dose of radiotherapy administered is a sub-therapeutic dose for treatment of the disorder with radiotherapy alone.

In some embodiments the total radiotherapy dose is, or is no greater than, 400 Gy, such as 2 to 400 Gy. For example, a dose of, or no greater than 200 Gy, 100 Gy, 80 Gy, 60 Gy, 40 Gy, 30 Gy, 24 Gy, 20 Gy, 18 Gy, 16 Gy, 15 Gy, 12 Gy, or 10 Gy.

In some embodiments the radiotherapy is adminstered as a single dose. However, radiation dosages may be fractionated and administered in sequence; for example, on consecutive days until the total desired radiation dose is delivered.

Fractionation of radiation doses is preferred. In some embodiments the radiotherapy is administered in two fractionated doses, or in at least two fractionated doses. In some embodiments the radiotherapy is administered in, or in at least, three, four, five, six, seven, eight, nine, ten, twelve, fifteen or twenty fractionated doses.

In some embodiments a fractionated dose is administered once daily (QD). In some embodiments a fractionated dose is administered once every other day (Q2D), once every third day (Q3D), twice weekly (2QW), once weekly (QW), once every two weeks (Q2W), or once every 3 weeks (Q3W).

In some embodiments the each fractionated dose is, or is no greater than, 20 Gy, such as 1 to 20 Gy. For example, a dose of, or no greater than, 15, 12, 10, 9, 8, 7, 6, 5, 4, 3, 2, or 1 Gy.

The CD25-ADC may be administered before the radiotherapy, simultaneous with the radiotherapy, or after the radiotherapy. Preferably the CD25-ADC is administered before the radiotherapy.

In some cases the combination of anti-CD25 ADC administration and radiotherapy administration are steps of the method of treatment claimed herein. In other cases the claimed method of treatment comprises only the administration of the anti-CD25 ADC, with the administration of radiotherapy to give the synergistic therapeutic effect described herein falling outside of the claimed method of treatment. In those other cases the synergistic combination may be made by selecting for treatment with the anti-CD25 ADC subjects who (i) have been treated with radiotherapy, and or (ii) will be treated with radiotherapy. As noted below, preferably the anti-CD25 ADC is administered before the radiotherapy so as to reduce the immune-suppressive activity of a population of regulatory immune cells prior to radiotherapy.

Radiosensitive

The terms “radiosensitivity” and “radiosensitive subject” are used herein to mean a subject that has increased sensitivity to the effects of radiation and radiotherapy.

The radiosensitivity of a subject may be defined by measuring the “mean breaks per metaphase (B/M)” parameter, as described in Schuster et al. 2018, BMC Geriatrics volume 18, Article number: 105. A subject having a B/M>0.6 is herein defined as a radiosensitive subject.

Combination with Checkpoint Inhibitors

In an aspect, the CD25-ADCs described herein are administered in combination with a checkpoint inhibitor.

The CD25-ADC may be administered before the checkpoint inhibitor, simultaneous with the checkpoint inhibitor, or after the checkpoint inhibitor.

The checkpoint inhibitor may be, for example, a PD1 antagonist, a PD-L1 antagonist, a GITR agonist, an OX40 agonist, or a CTLA-4 antagonist

PD1 Antagonists

Programmed death receptor I (PD1) is an immune-inhibitory receptor that is primarily expressed on activated T and B cells. Interaction with its ligands has been shown to attenuate T-cell responses both in vitro and in vivo. Blockade of the interaction between PD1 and one of its ligands, PD-L1, has been shown to enhance tumor-specific CD8+ T-cell immunity and may therefore be helpful in clearance of tumor cells by the immune system.

PD1 (encoded by the gene Pdcdl) is an Immunoglobulin superfamily member related to CD28, and CTLA-4. PD1 has been shown to negatively regulate antigen receptor signalling upon engagement of its ligands (PD-L1 and/or PD-L2). The structure of murine PD1 has been solved as well as the co-crystal structure of mouse PD1 with human PD-L1 (Zhang, X., et al., (2004) Immunity 20: 337-347; Lin, et al., (2008) Proc. Natl. Acad. Sci. USA 105: 30I I-6). PD1 and like family members are type I transmembrane glycoproteins containing an Ig Variable-type (V-type) domain responsible for ligand binding and a cytoplasmic tail that is responsible for the binding of signaling molecules. The cytoplasmic tail of PD1 contains two tyrosine-based signaling motifs, an ITIM (immunoreceptor tyrosine-based inhibition motif) and an ITSM (immunoreceptor tyrosine-based switch motif).

In humans, expression of PD1 (on tumor infiltrating lymphocytes) and/or PD-L1 (on tumor cells) has been found in a number of primary tumor biopsies assessed by immunohistochemistry. Such tissues include cancers of the lung, liver, ovary, cervix, skin, colon, glioma, bladder, breast, kidney, esophagus, stomach, oral squamous cell, urothelial cell, and pancreas as well as tumors of the head and neck (Brown, J. A., et al., (2003) J Immunol. I 70: 1257-1266; Dong H., et al., (2002) Nat. Med. 8: 793-800; Wintterle, et al., (2003) Cancer Res. 63: 7462-7467; Strome, S. E., et al., (2003) Cancer Res. 63: 6501-6505; Thompson, R. H., et al., (2006) Cancer Res. 66: 3381-5; Thompson, et al., (2007) Clin. Cancer Res. 13: I 757-61; Nomi, T., et al., (2007) Clin. Cancer Res. 13: 2151-7). More strikingly, PD-ligand expression on tumor cells has been correlated to poor prognosis of cancer patients across multiple tumor types (reviewed in Okazaki and Honjo, (2007) Int. Immunol. 19: 813-824).

To date, numerous studies have shown that interaction of PD1 with its ligands (PD-L1 and PD-L2) leads to the inhibition of lymphocyte proliferation in vitro and in vivo. Blockade of the PD1/PD-L1 interaction could lead to enhanced tumor-specific T-cell immunity and therefore be helpful in clearance of tumor cells by the immune system. To address this issue, a number of studies were performed. In a murine model of aggressive pancreatic cancer (Nomi, T., et al. (2007) Clin. Cancer Res. 13: 2151-2157), the therapeutic efficacy of PD1/PD-L1 blockade was demonstrated. Administration of either PD1 or PD-L1 directed antibody significantly inhibited tumor growth. Antibody blockade effectively promoted tumor reactive CD8+ T cell infiltration into the tumor resulting in the up-regulation of anti-tumor effectors including IFN gamma, granzyme Band perforin. Additionally, the authors showed that PD1 blockade can be effectively combined with chemotherapy to yield a synergistic effect. In another study, using a model of squamous cell carcinoma in mice, antibody blockade of PD1 or PD-L1 significantly inhibited tumor growth (Tsushima, F., et al., (2006) Oral Oneal. 42: 268-274).

“PD1 antagonist” means any chemical compound or biological molecule that stimulates an immune reaction through inhibition of PD1 signalling.

To examine the extent of enhancement of, e.g., PD1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) with PD1 inhibitors is advantageous, because on the one hand, the ADC will directly kill the FTP positive tumor cells, while on the other hand the PD1 inhibitor will engage the patient's own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD25(+) cells. Hence, the ADC will directly kill the tumor cells.

The resulting release of tumor associated antigens from cells that are killed with the PBD dimer will trigger the immune system, which will be further enhanced by the use of programmed cell death protein 1 (PD1) inhibitors, expressed on a large proportion of tumour infiltrating lymphocytes (TILs) from many different tumour types. Blockade of the PD1 pathway may enhance antitumour immune responses against the antigens released from the tumors killed by the ADC by diminishing the number and/or suppressive activity of intratumoral TReg cells.

The major function of PD1 is to limit the activity of T-cells at the time of an anti-inflammatory response to infection and to limit autoimmunity. PD1 expression is induced when T-cells become activated, and binding of one of its own ligands inhibits kinases involved in T-cell activation. Hence, in the tumor environment this may translate into a major immune resistance, because many tumours are highly infiltrated with TReg cells that probably further suppress effector immune responses. This resistance mechanism is alleviated by the use of PD1 inhibitors in combination with the ADC.

PD1 antagonists suitable for use as secondary agents in the present disclosure include:
a) a PD1 antagonist which inhibits the binding of PD1 to its ligand binding partners.
b) a PD1 antagonist which inhibits the binding of PD1 to PD-L1.
c) a PD1 antagonist which inhibits the binding of PD-1 to PDL2.
d) a PD1 antagonist which inhibits the binding of PD-1 to both PDLI and PDL2.
e) a PD1 antagonist of parts (a) to (d) which is an antibody.

Specific PD1 antagonists suitable for use as secondary agents in the present disclosure include:
a) pembrolizumab (brand name Keytruda)
   i. CAS Number→1374853-91-4
      (see http://www.cas.org/content/chemical-substances/faqs)
   ii. NCBI Pubchem reference→254741536
      (see https://pubchem.ncbi.nlm.nih.gov/)
   iii. DrugBank reference→DB09037
      (see https://www.drugbank.ca/)
   iv. Unique Ingredient Identifier (UNII)→DPT0O3T46P
      (see http://www.fda.gov/ForIndustry/DataStandards/SubstanceRegistration System-UniqueIngredientIdentifierUNII/default.htm)
b) nivolumab (brand name Opdivo)
   i. CAS Number→946414-94-4
      (see http://www.cas.org/content/chemical-substances/faqs)
   ii. DrugBank reference→DB09035
      (see https://www.drugbank.ca/)
c) MEDI0680 (formerly AMP-514)
   As described in WO2014/055648, WO2015/042246, WO2016/127052, WO2017/004016, WO2012/145493, U.S. Pat. No. 8,609,089, WO2016/007235, WO2016/011160; Int. J. Mol. Sci. 2016 July; 17(7): 1151, doi: 10.3390/ijms17071151; and Drug Discov Today, 2015 September; 20(9):1127-34. doi: 10.1016/j.drudis.2015.07.003.
   See also clinical trials NCT02271945 and NCT02013804 at https://clinicaltrials.gov/ct2/home
d) PDR001 (spartalizumab)
   i. CAS Number→1935694-88-4
      (see http://www.cas.org/content/chemical-substances/faqs)
   ii. Unique Ingredient Identifier (UNII)→QOG25L6Z8Z
      (see http://www.fda.gov/ForIndustry/DataStandards/SubstanceRegistration System-UniqueIngredientIdentifierUNII/default.htm)
   As described in WO2016/007235 and WO2016/011160
   NCI thesaurus code→C121625
      (see https://ncit.nci.nih.gov/ncitbrowser/)
e) Camrelizumab [INCSHR-1210] (Incyte)
   i. CAS Number→1798286-48-2
      (see http://www.cas.org/content/chemical-substances/faqs)

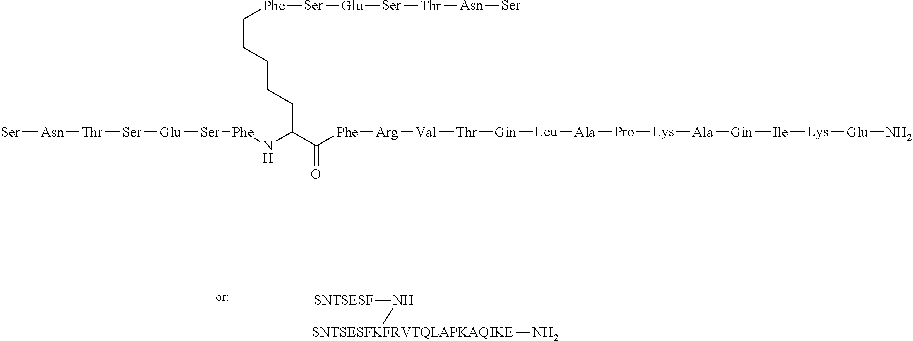

ii. Unique Ingredient Identifier (UNII)→73096E137E (see http://www.fda.gov/ForIndustry/DataStandards/SubstanceRegistration System-UniqueIngredientIdentifierUNII/default.htm)
f) AUNP12 (peptide) (Aurigene/PierreFabre)
   i. Disclosed in WO2011/161699 as SEQ ID NO:49 a.k.a. “compound 8”, see Example 2 on page 77 of the A2 publication of WO2011/161699.
   ii. CAS Number→1353563-85-5
      (see http://www.cas.org/content/chemical-substances/faqs)
g) Pidilizumab (CT-01 1)
   i. CAS Number→1036730-42-3
      (see http://www.cas.org/content/chemical-substances/faqs)
   ii. Unique Ingredient Identifier (UNII)→B932PAQ1BQ
      (see http://www.fda.gov/ForIndustry/DataStandards/SubstanceRegistration System-UniqueIngredientIdentifierUNII/default.htm)
h) Cemiplimab (formerly REGN-2810, SAR-439684)
   i. CAS Number→1801342-60-8
      (see http://www.cas.org/content/chemical-substances/faqs)
   ii. Unique Ingredient Identifier (UNII)→6QVL057INT
      (see http://www.fda.gov/ForIndustry/DataStandards/SubstanceRegistration System-UniqueIngredientIdentifierUNII/default.htm)
   As described in WO2016/007235
   NCI thesaurus code→C121540
      (see https://ncit.nci.nih.gov/ncitbrowser/)
i) BGB-A317 (Tislelizumab)
   i. As described in U.S. Pat. No. 9,834,606 B2
   ii. See clinical trial NCT03209973 (https://clinicaltrials.gov/)
   iii. NCI thesaurus code C121775
      (see https://ncit.nci.nih.gov/ncitbrowser/)
j) BGB-108
   See WO2016/000619 and U.S. Pat. No. 8,735,553
k) AMP-224

see clinical trial NCT02298946, https://clinicaltrials.gov/ct2/home In some embodiments, PD1 polypeptide corresponds to Genbank accession no. AAC51773, version no. AAC51773.1, record update date: Jun. 23, 2010 09:24 AM. In one embodiment, the nucleic acid encoding PD1 polypeptide corresponds to Genbank accession no. U64863, version no. U64863.1, record update date: Jun. 23, 2010 09:24 AM. In some embodiments, PD1 polypeptide corresponds to Uniprot/Swiss-Prot accession No. Q15116.

PD-L1 Antagonists

“PD-L1 antagonist” means any chemical compound or biological molecule that stimulates an immune reaction through inhibition of PD-L1 signalling.

To examine the extent of enhancement of, e.g., PD-L1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with PD-L1 inhibitors is advantageous because, on the one hand, the ADC will directly kill the FTP positive tumor cells while, on the other hand, the PD-L1 inhibitor will engage the patient's own immune system to eliminate the cancer cells.

Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor cells. The resulting release of tumor associated antigens from cells that are killed with the PBD dimer will trigger the immune system, which will be further enhanced by the use of programmed cell death protein 1 ligand inhibitors (PD-L1, aka B7-H1 or CD274).

PD-L1 is commonly upregulated on the tumour cell surface from many different human tumours. Interfering with the PD1 ligand expressed on the tumor will avoid the immune inhibition in the tumor microenvironment and therefore blockade of the PD1 pathway using PDL1 inhibitors may enhance antitumour immune responses against the antigens released from the tumors killed by the ADC.

Combining an ADC, which targets a first target protein (FTP) with PD1 inhibitors is advantageous, because on the one hand, the ADC will directly kill the FTP positive tumor cells, while on the other hand the PD1 inhibitor will engage the patient's own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD19(+) or CD22 (+) cells. Hence, the ADC will directly kill the tumor cells.

PD-L1 antagonists suitable for use as secondary agents in the present disclosure include PD-L1 antagonists that:
(a) are PD-L1 binding antagonists;
(b) inhibit the binding of PD-L1 to PD1;
(c) inhibit the binding of PD-L1 to B7-1;
(d) inhibit the binding of PD-L1 to both PD1 and B7-1;
(e) are anti-PD-L1 antibodies.

Specific PD-L1 antagonists suitable for use as secondary agents in the present disclosure include:
a) atezolizumab (MPDL3280A, brand name Tecentriq)
   i. CAS Number→1380723-44-3
      (see http://www.cas.org/content/chemical-substances/faqs)
   ii. DrugBank reference→DB11595
      (see https://www.drugbank.ca/)
   iii. Unique Ingredient Identifier (UNII)→52CMI0WC3Y
      (see http://www.fda.gov/ForIndustry/DataStandards/SubstanceRegistration System-UniqueIngredientIdentifierUNII/default.htm)
b) BMS-936559/MDX-1105
   I. CAS Number→1422185-22-5
      (see http://www.cas.org/content/chemical-substances/faqs)
   II. see clinical trial NCT02028403, https://clinicaltrials.gov/ct2/home
   III. See WO2007/005874 for antibody sequences, in articular the:

Antibody having:
a. VH CDR1 = DYGFS
b. VH CDR2 = WITAYNGNTNYAQKLQG
C. VH CDR3 = DYFYGMDV
d. VL CDR1 = RASQSVSSYLV
e. VL CDR2 = DASNRAT
f. VL CDR3 = QQRSNWPRT
Antibody having:
a. VH CDR1 = TYAIS
b. VH CDR2 = GIIPIFGKAHYAQKFQG
C. VH CDR3 = KFHFVSGSPFGMDV
d. VL CDR1 = RASQSVSSYLA
e. VL CDR2 = DASNRAT
f. VL CDR3 = QQRSNWPT
Antibody having:
a. VH CDR1 = SYDVH
b. VH CDR2 = WLHADTGITKFSQKFQG
C. VH CDR3 = ERIQLWFDY
d. VL CDR1 = RASQGISSWLA
e. VL CDR2 = AASSLQS
f. VL CDR3 = QQYNSYPYT

c) durvalumab/MEDI4736
   i. CAS Number→1428935-60-7 (see http://www.cas.org/content/chemical-substances/faqs)
   ii. Unique Ingredient Identifier (UNII)→28X28X9OKV (see
      http://www.fda.gov/ForIndustry/DataStandards/SubstanceRegistration System-UniqueIngredientIdentifierUNII/default.htm)
   iii. VH sequence

EVQLVESGGGLVQPGGSLRLSCAASGFTFSRYWMSWRQAPGKGLEVANI
KQDGSEKYYVDSVKGRFTISRDNAKNSLYLQMNSLRAEDTAVYYCAREG
GWFGELAFDYWGQGTLVTVSS
iv. VL sequence
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK

d) Avelumab/MSB0010718C
   i. CAS Number→1537032-82-8
      (see http://www.cas.org/content/chemical-substances/faqs)
   ii. Unique Ingredient Identifier (UNII)→KXG2PJ551I
      (see http://www.fda.gov/ForIndustry/DataStandards/SubstanceRegistration System-UniqueIngredientIdentifierUNII/default.htm)

In some embodiments, PD-L1 polypeptide corresponds to Genbank accession no. AAF25807, version no. AAF25807.1, record update date: Mar. 10, 2010 10:14 PM. In one embodiment, the nucleic acid encoding PD1 polypeptide corresponds to Genbank accession no. AF177937, version no. AF177937.1, record update date: Mar. 10, 2010 10:14 PM. In some embodiments, PD1 polypeptide corresponds to Uniprot/Swiss-Prot accession No. Q9NZQ7.

GITR Agonists

The term “glucocorticoid-induced TNF receptor” (abbreviated herein as “GITR”), also known as TNF receptor superfamily 18 (TNFRSF18, CD357), TEASR, and 312C2, as used herein, refers to a member of the tumor necrosis factor/nerve growth factor receptor family. GITR is a 241 amino acid type I transmembrane protein characterized by three cysteine pseudo-repeats in the extracellular domain and specifically protects T-cell receptor induced apoptosis, although it does not protect cells from other apoptotic signals, including Fas triggering, dexamethasone treatment, or UV irradiation (Nocentini, G., et al. (1997) Proc. Natl. Acad. Sci. USA 94:6216-622).

GITR activation increases resistance to tumors and viral infections, is involved in autoimmune/inflammatory processes and regulates leukocyte extravasation (Nocentini supra; Cuzzocrea, et al. (2004) J Leukoc. Biol. 76:933-940; Shevach, et al. (2006) Nat. Rev. Immunol. 6:613-6I8; Cuzzocrea, et al. (2006) J Immunol. I 77:63I-64I; and Cuzzocrea, et al. (2007) FASEB J 2I: I I7-I29). In tumor mouse models, agonist GITR antibody, DTA-I, was combined with an antagonist CTLA-4 antibody, and showed synergistic results in complete tumor regression of advanced stage tumors in some test group mice (Ko, et al. (2005) J Exp. Med. 7:885-891).

The nucleic acid and amino acid sequences of human GITR (hGITR), of which there are three splice variants, are known and can be found in, for example GenBank Accession Nos. gi:40354198 [NM_005092.3], gi:23238190 [NM_004195.3], gi:23238193 [NM_148901.1], and gi:23238196 [NM_148902.1].

“GITR agonist” means any chemical compound or biological molecule that stimulates an immune reaction through activation of GITR signalling. Also contemplated are soluble GITR-L proteins, a GITR binding partner.

To examine the extent of enhancement of, e.g., GITR activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with GITR agonists is advantageous, because on the one hand the ADC will directly kill the FTP positive tumor cells, while on the other hand the GITR agonist will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a GITR agonist.

GITR (Glucocorticoid-Induced TNFR-Related protein) is expressed transiently on activated T-cells and expressed constitutively at high levels on T-regs with further induction following activation. GITR ligation via its ligand GITRL stimulates both proliferation and function of both effector and regulatory CD4+ T cells. This promotes T-cell survival, and differentiation into effector cells, while abrogating suppression. Therefore it will be beneficial to target a FTP(+) tumor with the ADC, causing the antigenic cell death, while the GITR agonist induces a stronger, durable immune response.

Specific GITR agonists suitable for use as secondary agents in the present disclosure include:
a) MEDI1873, a GITR ligand fusion protein developed by MedImmune
   See WO2016/196792, US20160304607
   NCI thesaurus code→C₁₂₄₆₅₁
      (see https://ncit.nci.nih.gov/ncitbrowser)
   See also clinical trial NCT023126110 at https://clinicaltrials.gov/ct2/home
   See Tigue N J, Bamber L, Andrews J, et al. MEDI1873, a potent, stabilized hexameric agonist of human GITR with regulatory T-cell targeting potential. Oncoimmunology. 2017; 6(3):e1280645. doi:10.1080/2162402X.2017.1280645.
b) INCAGN1876, is an agonist antibody targeting the glucocorticoid-induced TNFR-related protein, or GITR. Discovered during a collaboration with Ludwig Cancer Research. INCAGN1876 is being co-developed with
See clinical trials NCT02583165 and NCT03277352 at https://clinicaltrials.gov/ct2/home
c) TRX518, a humanized aglycosylated (Fc disabled) IgG1 anti-GITR mAb with immune-modulating activity developed by Leap Therapeutics
   See WO2006/105021 for sequences 58, 60-63; and EP2175884 sequences 1-7:

VL comprising the sequence (CDR underline):
EIVMTQSPATLSVSPGERATLSCKASQNVGTNVAWYQQKPGQA
PRLLIYSASYRYSGIPARFSGSGSGTEFTLTISSLQSEDFA
VYYCQQYNTDPLTFGGGTKVEIK
VH comprising the sequence (CDR underline):
QVTLRESGPALVKPTQTLTLTCTFSGFSLSTSGMGVGWIRQPPG
KALEWLAHIWWDDDKYY [symbol] PSLKSRLTISKDTSKNQWLTMTNM
DPVDTATYYCARTRRYFPFAYWGQGTLVTVS
QVTLRESGPALVKPTQTLTLTCTFSGFSLSTSGMGVGWIRQPPG
KALEWLAHIWWDDDKYY [symbol] PSLKSRLTISKDTSKNQVVLTMTNM
DPVDTATYYCARTRRYFPFAYWGQGTLVTVS

See clinical trials NCT01239134 and NCT02628574 at https://clinicaltrials.gov/ct2/home
   NCI thesaurus code→C95023
      (see https://ncit.nci.nih.gov/ncitbrowser)
d) GWN323, an anti-GITR agonistic monoclonal antibody, which activates GITRs found on multiple types of T-cells. GWN323 is developed by Novartis
   See WO2016/196792
   NCI thesaurus code→C128028 (see https://ncit.nci.nih.gov/ncitbrowser)
   See clinical trial NCT02740270 at https://clinicaltrials.gov/ct2/home
e) MK-1248, a humanized IgG4 anti-human glucocorticoid-induced tumor necrosis factor receptor (GITR) agonistic monoclonal antibody (MoAb) with significantly reduced effector function
   See clinical trial NCT02553499 at https://clinicaltrials.gov/ct2/home
   MK-1248 has the same CDR as MK4166 (see Sukumar et al., Cancer Res. 2017)
f) MK-4166, a humanized IgG1 anti-human glucocorticoid-induced tumor necrosis factor receptor (GITR) agonistic monoclonal antibody (MoAb) with potential immunomodulating activity (see Sukumar et al., Cancer Res. 2017).
   See clinical trial NCT02132754 at https://clinicaltrials.gov/ct2/home
   See Sukumar, et al., (2017), Cancer Research. 77. canres.1439.2016. 10.1158/0008-5472.CAN-16-1439.
   NCI thesaurus code C116065
      (see https://ncit.nci.nih.gov/ncitbrowser/)
g) BMS-986156, An anti-human glucocorticoid-induced tumor necrosis factor receptor (GITR; tumor necrosis factor superfamily member 18; TNFRSF18; CD357) agonistic monoclonal antibody
   See clinical trial NCT02598960 at https://clinicaltrials.gov/ct2/home
   NCI thesaurus code C132267
      (see https://ncit.nci.nih.gov/ncitbrowser/)

Sequences of agonist anti-GITR antibodies are provided in WO2011/028683 and WO2006/105021.

In some embodiments, GITR polypeptide corresponds to Genbank accession no. AAD22635, version no. AAD22635.1, record update date: Mar. 10, 2010 09:42 PM. In one embodiment, the nucleic acid encoding GITR polypeptide corresponds to Genbank accession no. AF125304, version no. AF125304.1, record update date: Mar. 10, 2010 09:42 PM. In some embodiments, GITR polypeptide corresponds to Uniprot/Swiss-Prot accession No. Q9Y5U5.

OX40 Agonists

OX40 (CD134; TNFRSF4) is a member of the TNFR super-family and is expressed by CD4 and CD8 T cells during antigen-specific priming. OX40 expression is largely transient following TCR/CD3 cross-linking, and by the presence of inflammatory cytokines. In the absence of activating signals, relatively few mature T cell subsets express OX40 at biologically relevant levels. Generating optimal “killer” CD8 T cell responses requires T cell receptor activation plus co-stimulation, which can be provided through ligation of OX40 using a OX40 agonist. This activating mechanism augments T cell differentiation and cytolytic function leading to enhanced anti-tumor immunity. Therefore it will be beneficial to target a FTP(+) tumor with the ADC, causing the antigenic cell death, while the OX40 agonist induces a stronger, durable immune response.

The OX40 agonist may be selected from the group consisting of an OX40 agonist antibody, an OX40L agonist fragment, an OX40 oligomeric receptor, and an OX40 immunoadhesin. In some embodiments, the OX40 binding agonist is a trimeric OX40L-Fc protein.

In some embodiments, the OX40 binding agonist is an OX40L agonist fragment comprising one or more extracellular domains of OX40L. In some embodiments, the OX40 binding agonist is an OX40 agonist antibody that binds human OX40. In some embodiments, the OX40 agonist antibody depletes cells that express human OX40. In some embodiments, the OX40 agonist antibody depletes cells that express human OX40 in vitro. In some embodiments, the cells are CD4+ effector T cells. In some embodiments, the cells are Treg cells. In some embodiments, the depleting is by ADCC and/or phagocytosis. In some embodiments, the depleting is by ADCC. In some embodiments, the OX40 agonist antibody binds human OX40 with an affinity of less than or equal to about 1 nM. In some embodiments, the OX40 agonist antibody increases CD4+ effector T cell proliferation and/or increasing cytokine production by the CD4+ effector T cell as compared to proliferation and/or cytokine production prior to treatment with anti-human OX40 agonist antibody. In some embodiments, the cytokine is gamma interferon. In some embodiments, the OX40 agonist antibody increases memory T cell proliferation and/or increasing cytokine production by the memory cell. In some embodiments, the cytokine is gamma interferon. In some embodiments, the OX40 agonist antibody inhibits Treg function. In some embodiments, the OX40 agonist antibody inhibits Treg suppression of effector T cell function. In some embodiments, effector T cell function is effector T cell proliferation and/or cytokine production. In some embodiments, the effector T cell is a CD4+ effector T cell. In some embodiments, the OX40 agonist antibody increases OX40 signal transduction in a target cell that expresses OX40. In some embodiments, OX40 signal transduction is detected by monitoring NFkB downstream signalling.

“OX40 agonist” means any chemical compound or biological molecule that stimulates an immune reaction through inactivation of OX40 signalling.

To examine the extent of enhancement of, e.g., OX40 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with OX40 agonists is advantageous, because on the one hand the ADC will directly kill the FTP positive tumor cells, while on the other hand the OX40 agonist will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a OX40 agonist.

Specific OX40 agonists suitable for use as secondary agents in the present disclosure include:
a) MEDI0562 (aka Tavolixizumab, Tavolimab)
   i. CAS Number→1635395-25-3
      (see http://www.cas.org/content/chemical-substances/faqs)
   ii. Unique Ingredient Identifier (UNII)→4LU9B48U4D
      (see http://www.fda.gov/ForIndustry/DataStandards/SubstanceRegistration System-UniqueIngredientIdentifierUNII/default.htm)
   See clinical trial NCT02318394 at https://clinicaltrials.gov/ct2/home
   As described in WO2015/095423, WO2015/153514, WO2016/073380 & WO2016/081384
   NCI thesaurus code→C120041
      (see https://ncit.nci.nih.gov/ncitbrowser/)

Heavy Chain sequence:
QVQLQESGPGLVKPSQTLSLTCAVYGGSFSSGYWN
WIRKHPGKGLEYIGYISYNGITYHNPSLKSRITIN
RDTSKNQYSLQLNSVTPEDTAVYYCARYKYDYDGG
HAMDYWGQGTLVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKPS
NTKVDKRVEPKSCDKTHTCPPCPAPELLGGPSVFL
FPPKPKDTLMISRTPEVTCVVVDVSHEDPEVKFNWY
VDGVEVHNAKTKPREEQYNSTYRVVSVLTVLHQDWL
NGKEYKCKVSNKALPAPIEKTISKAKGQPREPQVY
TLPPSREEMTKNQVSLTCLVKGFYPSDIAVEWESN
GQPENNYKTTPPVLDSDGSFFLYSKLTVDKSRWQQ
GNVFSCSVMHEALHNHYTQKSLSLSPG
Light chain sequence:
DIQMTQSPSSLSASVGDRVTITCRASQDISNYLNW
YQQKPGKAPKLLIYYTSKLHSGVPSRFSGSGSGTD
YTLTISSLQPEDFATYYCQQGSALPWTFGQGTKVE
IKRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFN
RGEC

b) MEDI6383 (Efizonerimod alfa)
   i. CAS Number→1635395-27-5
      (see http://www.cas.org/content/chemical-substances/faqs)
   ii. Unique Ingredient Identifier (UNII)→1MH7C2X8KE
      (see http://www.fda.gov/ForIndustry/DataStandards/SubstanceRegistration System-UniqueIngredientIdentifierUNII/default.htm)
   See clinical trial NCT02221960 at https://clinicaltrials.gov/ct2/home
   As described in WO2015/095423, WO2016/081384, and WO2016/189124
   NCI thesaurus code→C118282
      (see https://ncit.nci.nih.gov/ncitbrowser/)
   Amino acid sequence Se ID no. 17 from WO2016/189124):

ESKYGPPCPPCPAPEFLGGPSVFLFPPKPKDTLMI
SRTPEVTCVVDVSQEDPEVQFNWYVDGVEVHNAKT
KPREEQFNSTYRVVSVLTVLHQDWLNGKEYKCKVSN
KGLPSSIEKTISKAKGQPREPQVYTLPPSQEEMTK
NQVSLTCLVKGFYPSDIAVEWESNGQPENNYKTTP
PVLDSDGSFFLYSRLTVDKSRWQEGNVFSCSVMHE
ALHNHYTQKSLSLSLGKDQDKIEALSSKVQQLERS
IGLKDLAMADLEQKVLEMEASTQVSHRYPRIQSIK
VQFTEYKKEKGFILTSQKEDEIMKVQNNSVIINCD
GFYLISLKGYFSQEVNISLHYQKDEEPLFQLKKVR
SVNSLMVASLTYKDKVYLNVTTDNTSLDDFHVNGG
ELILIHQNPGEFCVL

c) MOXR0916 (also known as RG7888, Pogalizumab), a humanized anti-OX40 monoclonal antibody
   i. CAS Number→1638935-72-4
      (see http://www.cas.org/content/chemical-substances/faqs)
   ii. Unique Ingredient Identifier (UNII)→C78148TF1D
      (see http://www.fda.gov/ForIndustry/DataStandards/SubstanceRegistration System-UniqueIngredientIdentifierUNII/default.htm)
   iii. NCI thesaurus code→C121376
      (see https://ncit.nci.nih.gov/ncitbrowser/)
d) OX40mAb24 (9B12)
   i. OX40mAb24 is a humanised version of 9B12. 9B12 is a murine IgGI, anti-OX40 mAb directed against the extracellular domain of human OX40 (CD134) (Weinberg, A. D., et al. J Immunother 29, 575-585 (2006)).
   ii. See WO2016/057667 Seq ID no. 59 for OX40mAb24 VH sequence, no. 29 for VL sequence (no. 32 is an alternative VL):

VH sequence
QVQLQESGPGLVKPSQTLSLTCAVYGGSFSSGYWN
WIRKHPGKGLEYIGYISYNGITYHNPSLKSRITIN
RDTSKNQYSLQLNSVTPEDTAVYYCARYKYDYDGG
HAMDYWGQGTLVTVSS
VL sequence
DIQMTQSPSSLSASVGDRVTITCRASQDISNYLNW
YQQKPGKAPKLLIYYTSKLHSGVPSRFSGSGSGTD
YTLTISSLQPEDFATYYCQQGSALPWTFGQGTKVE
IK

e) INCAGN1949
   i. See Gonzalez et al. 2016, DOI: 10.1158/1538-7445.AM2016-3204
   ii. See clinical trial NCT02923349 at https://clinicaltrials.gov/ct2/home
   iii. Antibody sequences are disclosed in WO2016/179517 A1:
   i. In particular an antibody comprising the sequences:

VH CDR1 → GSAMH
VH CDR2 → RIRSKANSYATAYAASVKG
VH CDR3 → GIYDSSGYDY
VL CDR1 → RSSQSLLHSNGYNYLD
VL CDR2 → LGSNRAS
VL CDR3 → MQALQTPLT

ii. Such as, an antibody comprising the sequences:

VH →
EVQLVESGGGLVQPGGSLKLSCAASGFTFSGSAMHWWRQA
SGKGLEWGRIRSKANSYATAYAASVKGRFTISRDDSKNTA
YLQMNSLKTEDTAVYYCTSGIYDSSGYDYWGQGTLVTVSS
VL →
DIVMTQSPLSLPVTPGEPASISCRSSQSLLHSNGYNYLDW
YLQKPGQSPQLLIYLGSNRASGVPDRFSGSGSGTDFTLKI
SRVEAEDVGVYYCMQALQTPLTFGGGTKVEIK

g) GSK3174998, a humanized IgG1 agonistic anti-OX40 monoclonal antibody (mAb)
See clinical trial NCT02528357 at https://clinicaltrials.gov/ct2/home
h) PF-04518600 (PF-8600) is an investigational, fully human, monoclonal antibody (mAb) that targets OX40 protein
See patent WO 2017/130076 A1
See clinical trial NCT02315066 at https://clinicaltrials.gov/ct2/home-NCI thesaurus code→C121927
   (see https://ncit.nci.nih.gov/ncitbrowser/)

In some embodiments, OX40 polypeptide corresponds to Genbank accession no. CAA53576, version no. CAA53576.1, record update date: Feb. 2, 2011 10:10 AM. In one embodiment, the nucleic acid encoding OX40 polypeptide corresponds to Genbank accession no. X75962, version no. X75962.1, record update date: Feb. 2, 2011 10:10 AM. In some embodiments, OX40 polypeptide corresponds to Uniprot/Swiss-Prot accession No. P43489.

CTLA Antagonist

CTLA4 (CD152) is expressed on activated T cells and serves as a co-inhibitor to keep T cell responses in check following CD28-mediated T cell activation. CTLA4 is believed to regulate the amplitude of the early activation of naive and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signalling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

“CTLA4 agonist” means any chemical compound or biological molecule that stimulates an immune reaction through inhibition of CTLA4 signalling.

To examine the extent of enhancement of, e.g., CTLA4 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with CTLA4 inhibitors is advantageous, because on the one hand, the ADC will directly kill the FTP positive tumor cells, while on the other hand the CTLA4 inhibitor will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of CTLA4 inhibitors expressed on a large proportion of tumour infiltrating lymphocytes (TILs) from many different tumour types.

The major function of CTLA4 (CD152) is to regulate the amplitude of the early stages of T cell activation, and as such it counteracts the activity of the T cell co-stimulatory receptor, CD28, In the tumor microenvironment. Blockade of the CTLA4 pathway may therefore enhance enhancement of effector CD4+ T cell activity, while it inhibits TReg cell-dependent immunosuppression. Therefore it will be beneficial to target a FTP(+) tumor with the ADC, causing the antigenic cell death, while the CTLA4 blockade induces a stronger immune, durable response.

Specific CTLA4 antagonists suitable for use as secondary agents in the present disclosure include:
a) ipilimumab
   i. CAS Number→477202-00-9
      (see http://www.cas.org/content/chemical-substances/faqs)
   ii. Unique Ingredient Identifier (UNII)→6T8C155666
      (see http://www.fda.gov/ForIndustry/DataStandards/SubstanceRegistration System-UniqueIngredientIdentifierUNII/default.htm)
b) Tremelimumab
   i. CAS Number→745013-59-6
      (see http://www.cas.org/content/chemical-substances/faqs)
   ii. Unique Ingredient Identifier (UNII)→QEN1X95CIX
      (see http://www.fda.gov/ForIndustry/DataStandards/SubstanceRegistration System-UniqueIngredientIdentifierUNII/default.htm)

iii.
VH sequence
[SEQ ID NO. 1]
GWVQPGRSLRLSCAASGFTFSSYGMHWRQAPGKGLEWV
AVIWYDGSNKYYADSVKGRFTISRDNSKNTLYLQMNSL
RAEDTAVYYCARDPRGATLYYYYYGMDVWGQGTTVTVS
SASTKGPSVFPLAPCSRSTSESTAALGCLVKDYFP
EPVTVSWNSGALTSGVH
iv.
VL sequence
[SEQ ID NO. 2]
PSSLSASVGDRVTITCRASQSINSYLDWYQQKPGKAPK
LLIYAASSLQSGVPSRFSGSGSGTDFTLTISSLQPEDF
ATYYCQQYYSTPFTFGPGTKVEIKRTVAAPSVFIFPP
SDEQLKSGTASVVCLLNNFYPREAKV

In some embodiments, CTLA polypeptide corresponds to Genbank accession no. AAL07473, version no. AAL07473.1, record update date: Mar. 11, 2010 01:28 AM. In one embodiment, the nucleic acid encoding CTLA4 polypeptide corresponds to Genbank accession no. AF414120, version no. AF414120.1, record update date: Mar. 11, 2010 01:28 AM. In some embodiments, OX40 polypeptide corresponds to Uniprot/Swiss-Prot accession No. P16410.

Treated Disorders

The therapies described herein include those that induce or enhance a subject's immune response in combination with radiotherapeutic stimulation of immunogenicity. In particular, in certain aspects the therapies include treating a disorder by inducing or enhancing the immune response of a subject against an antigen associated with the disorder.

In some aspects the therapies described herein enhance or induce an immune response by targeting immune regulatory cells with an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to the targeted tissue whereby greater selectivity, i.e. a lower efficacious dose, may be achieved. The targeting of immune regulatory cells in this manner allows for a reduction in the negative regulation of the subject's immune responses to an existing or newly presented antigen.

The methods described herein may be used in combination with other immune response stimulating agents in order to further enhance and/or induce an immune response. This approach is expected to have utility in, for example, highly immunosuppressive circumstances that are not overcome through use of a single immunostimulating agent/method.

For example, molecules such as CD3/DAA bi-specific T-cell engagers (BiTEs) function to direct cytotoxic T-cells' cell-killing activity against target cells bearing a DAA. BiTEs therefore stimulate an immune reaction against DAA bearing cells (see Zimmerman et al., International Immunology, Volume 27, Issue 1, January 2015, Pages 31-37). A well known example of a BiTE is Blinatumomab—a CD3/CD19 BiTE used to treat CD19+ve B-cell linage cancers such as CLL and ALL (see Robinson et al. Blood 2018:blood-2018-02-830992).

However, the immune reaction stimulated by a BiTE may still be suppressed by, for example: (1) high levels of immune suppressive cells (see Ellerman, Methods, Volume 154, 1 Feb. 2019, Pages 102-117), and/or (2) activation of immune regulatory cells by the BiTE itself (see Koristka et al. 2015, Oncoimmunology. 2015 March; 4(3): e994441). Accordingly, the methods for reducing the immune-suppressive activity of a population of immune regulatory cells described herein may be usefully combined with, for example, BiTEs to further enhance the immune response against DAA bearing target cells. Such a combination will have particular utility in patient populations where the efficacy of a first immune stimulatory agent/method (eg. BiTE) is inhibited or reduced by the immune-suppressive activity of a population of CD25+ve immune regulatory cells such as Tregs.

In some aspects the therapies described herein enhance or induce an immune response by directly killing target cells through cytotoxic ADC binding to the target cells and/or, through a ‘bystander effect’, indirectly killing target cells in the proximity of cells that are directly bound by the cytotoxic ADC (see, for example, WO/2016/083468). The killing of target cells causes the release of target antigens, ‘stranger signals’, and/or ‘danger signals’ into the extracellular environment where they can interact with and stimulate a subject's immune system (see, for example, Virgil E J C Schijns & Ed C Lavelle (2011) Expert Review of Vaccines, 10:4, 539-550).

Thus, in one aspect, the present disclosure provides a method of inducing or enhancing an immune response in a subject, the method comprising the step of administering to the subject a CD25-ADC in combination with radiotherapy. The induction or enhancement of immune response may be due to the reduction in the immune-suppressive activity of an immune regulatory cell population combined with immunogenic cell death of the tissue targeted by radiotherapy, as defined herein.

One of ordinary skill in the art is readily able to determine whether or not a candidate therapy treats a particular disorder characterized by a disorder-associated antigen (DAA). For example, assays which may conveniently be used to assess the activity offered by a particular compound are described below.

The therapies described herein may be used to treat a proliferative disorder. The term “proliferative disorder” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocytoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders of interest include, but are not limited to, Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12].

Proliferative disorders of particular interest include those associated with elevated numbers of regulatory immune cells, such as Treg cells. These include chronic lymphatic lymphoma (CLL), T-cell Acute Lymphoblastic Leukaemia (T-ALL), and B-cell non-Hodgkin's Lymphoma, such as Acute Myeloid Leukaemia (AML) [Niedzwiecki et al., J.Immun.R., Vol. 2018, Article ID 1292404].

Classical Hodgkins lymphoma includes the subtypes nodular sclerosing, lymphocyte predominant, lymphocyte depleted and mixed cellularity. The Hodgkins lymphoma subtype may not be defined. In certain aspects, the patients tested according to the methods here have Hodgkins lymphoma of the nodular sclerosing and mixed cellularity subtypes.

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve Tregs.

The target neoplasm or neoplastic cells may be all or part of a solid tumour.

Solid tumors may be neoplasms, including non-haematological cancers, comprising or composed of CD25+ve neoplastic cells. Solid tumors may be neoplasms infiltrated with CD25+ve cells, such as CD25+ve Tregs; such solid tumours may lack expression of CD25 (that is, comprise or be composed of CD25−ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

The solid tumour may be a tumour with low levels of infiltrating T-cells, such as infiltrating regulatory T-cells.

Less preferably, the solid tumour may be a tumour that is not associated or infiltrated with CD25+ve cells, such as CD25+ve Tregs.

In some embodiments the high/low/no infiltrating T-cell status of a tumour is determined by measuring the ratio of T-regulatory cells/T-effector using, for example, FACS analysis of T-cells in a sample. In some embodiments the level of infiltrating T-cells is determined to be ‘high’ if the ratio of T-regulatory cells/T-effector is at least 20. In some embodiments the level of infiltrating T-cells is determined to be ‘low’ if the ratio of T-regulatory cells/T-effector is less than 20.

The neoplasm or neoplastic cells may be all or part of an established tumour. An ‘established tumour’ as described herein may be, for example, a tumour such as a solid tumour diagnosed or identified in a naïve subject.

In some cases the naïve subject is a subject that has not yet been treated to reduce the immune-suppressive activity of an immune regulatory cell population, as defined herein; for example; treated with an anti-CD25 antibody or a CD25-ADC. In some cases the naïve subject is a subject that has not yet been treated with ADCx25, as defined herein.

The neoplasm or neoplastic cells may be a circulating tumour or circulating tumour cells (CTC; Gupta et al. 2006, Cell. 127 (4): 679-95; Rack et al., 2014. Journal of the National Cancer Institute. 106 (5)). The CTCs may be, or comprise, metastatic cells (i.e. CTCs capable of establishing metastatic tumours in a subject).

In some cases the treated tumour is a solid tumour that is directly targeted by radiotherapy. That is, the treatment with radiotherapy and the anti-CD25 ADC stimulates an immune response against the same tumour that was directly targeted by the radiotherapy. In some cases the treated tumour is remote from the radiotherapy administration site. That is, the treatment with radiotherapy and the anti-CD25 ADC stimulates an immune response against a tumour that was not directly targeted by the radiotherapy (ie. the treated tumour was not irradiated). In these cases the tumour may be therapeutic effect is believed to arise entirely from immune stimulation at the radiotherapy site, with subsequent remote action of activated immune cells. Accordingly, the present methods allow for effective treatment of tumours that are, for example, located in areas which makes them unsuitable for treatment with direct radiotherapy.

It is contemplated that the therapies of the present disclosure may be used to treat various proliferative disorders. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

It is contemplated that the therapies of the present disclosure may be used to treat any proliferative disorder that is characterized by a disease-associated antigen (DAA). Typically, a DAA is an antigen that is either: (1) expressed only on diseased cells, or (2) expressed at higher levels by diseased cells as compared to normal cells. Often a DAA will be an antigen present on the surface of a diseased cell.

Subject Selection

In certain aspects, the subjects are selected as suitable for treatment with the treatments before the treatments are administered. In some aspects the treatment methods described herein include the step of selecting suitable subjects. In some aspects the treatment methods described herein treat subjects that have been previously selected as suitable for treatment.

As used herein, subjects who are considered suitable for treatment are those subjects who are expected to benefit from, or respond to, the treatment. Subjects may have, be suspected of having, have been diagnosed with, or be at risk of, a disorder characterized by a disease-associated antigen (DAA) as described herein.

In some aspects the treated subject has been selected for treatment on the basis that the subject has, is suspected of having, has been diagnosed with, or is at risk of, a disorder characterized by a disorder-associated antigen (DAA).

In some aspects the subject is: (1) selected for treatment on the basis that the subject has, is suspected of having, has been diagnosed with, or is at risk of, a disorder characterized by a disorder-associated antigen (DAA); then (2) treated with a CD25-ADC as described herein.

In particular, the disorder characterized by a disorder-associated antigen (DAA) may be solid tumour as described herein.

In some aspects, subjects are selected fro treatment if they are radiosensitive, since the methods described herein allow for effective treatment using reduced radiation doses.

In some aspects, subjects are selected for treatment if they have multiple tumours, at least one of which is treatable with the methods described herein. In some of these cases, the subjects may also have one or more tumours (eg. metastatic tumours) that are inoperable, untreatable by conventional radiotherapy, and/or otherwise refractory to conventional treatments.

In some aspects, subjects are selected on the basis of the amount or pattern of expression of CD25. In some aspects, the selection is based on expression of CD25 at the cell surface in a tissue or structure of interest. So, in some cases, subjects are selected on the basis they have, or are suspected of having, are at risk of having, or have received a diagnosis of a proliferative disease characterized by the presence of a neoplasm comprising or associated with cells having surface expression of CD25. The neoplasm may be composed of cells having surface expression of CD25.

In some aspects, subjects are selected on the basis they have a neoplasm comprising both CD25+ve and CD25−ve cells. The neoplasm may be composed of CD25−ve neoplastic cells, optionally wherein the CD25−ve neoplastic cells are associated with CD25+ve non-neoplastic cells such as CD25+ve Tregs. The neoplasm or neoplastic cells may be all or part of a solid tumour. The solid tumour may be partially or wholly CD25−ve, and may be infiltrated with CD25+ve cells, such as CD25+ve Tregs. In preferred aspects, the solid tumour is associated with high-levels of CD25+ve infiltrating cells, such as Treg cells. In some aspects, the solid tumour is associated with low-levels of CD25+ve infiltrating cells, such as Treg cells. In some aspects, the solid tumour is not associated with CD25+ve infiltrating cells, such as Treg cells; for example, the levels of CD25+ve cells may be below the detection limit.

In some cases, expression of CD25 in a particular tissue of interest is determined. For example, in a sample of tumor tissue. In some cases, systemic expression of CD25 is determined. For example, in a sample of circulating fluid such as blood, plasma, serum or lymph.

In some aspects, the subject is selected as suitable for treatment due to the presence of CD25 expression in a sample. In those cases, subjects without CD25 expression may be considered not suitable for treatment.

In other aspects, the level of CD25 expression is used to select a subject as suitable for treatment. Where the level of expression of the target is above a threshold level, the subject is determined to be suitable for treatment.

In some aspects, an subject is indicated as suitable for treatment if cells obtained from the tumour react with antibodies against CD25 as determined by IHC.

In some aspects, a subject is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express CD25. In some aspects disclosed herein, a subject is determined to be suitable for treatment if at least at least 5% of the cells in the sample express CD25.

In some cases the combination of anti-CD25 ADC administration and radiotherapy administration are steps of the method of treatment claimed herein. In other cases the claimed method of treatment comprises only the administration of the anti-CD25 ADC, with the administration of radiotherapy to give the synergistic therapeutic effect described herein falling outside of the claimed method of treatment. In those other cases the synergistic combination may be made by selecting for treatment with the anti-CD25 ADC subjects who (i) have been treated with radiotherapy, and or (ii) will be treated with radiotherapy. As noted elsewhere herein, preferably the anti-CD25 ADC is administered before the radiotherapy (ie. case (i)) so as to reduce the immune-suppressive activity or size of a population of regulatory immune cells prior to radiotherapy.

Accordingly, the present disclosure provides a method of inducing or enhancing an immune response against a disorder in a subject, the method comprising:
(a) selecting for treatment a subject who has, is, or will be treated with radiotherapy; and
(b) administering to the subject an effective amount of an anti-CD25 ADC.

The present disclosure also provides a method for treating a disorder in an subject, the method comprising:
(a) selecting for treatment a subject who has, is, or will be treated with radiotherapy; and
(b) administering to the subject an effective amount of an anti-CD25 ADC.

Preferably the subjects are selected for treatment by (i) identifying a subject who has, is, or will be treated with radiotherapy, then (ii) selecting the subject for treatment if they have, are, or will be treated with radiotherapy.

The selection of subjects who have, are, or will be treated with radiotherapy is with the intention of achieving the synergistic therapeutic effect between the radiotherapy and CD25-ADC reported herein. Accordingly, the subjects are preferably only selected for treatment with the CD25-ADC if the radiotherapy has, or will be, administered such that the synergistic therapeutic effect is expected to be obtained. In particular, subjects are preferably only selected for treatment with the CD25-ADC if the timing of the CD25-ADC and radiotherapy administration is, or is expected to be, as described above in the section entitled ‘Sequence of administration’.

Treatment of Established Tumours & Reduction of Metastatic Tumours

The methods described and exemplified herein have been shown to be effective at treating established tumours in naïve subjects as well as the reduction or prevention of metastatic tumours in previously treated subjects.

Accordingly, in some aspects a subject is selected for treatment if they have, are suspected of having, have been diagnosed with, or are at risk of, an established tumour, such as an established solid tumour. An ‘established tumour’ as described herein may be, for example, a tumour diagnosed or identified in a naïve subject.

In some cases a naïve subject is a subject that has not yet been treated to reduce the immune-suppressive activity of an immune regulatory cell population, as defined herein; for example; treated with an anti-CD25 antibody or a CD25-ADC. In some cases a naïve subject is a subject that has not yet been treated with ADCx25, as defined herein.

In some cases, an ‘established tumour’ as described herein may be a relapsed or resistant tumour. For example, a relapsed tumour may a new or growing tumour identified or diagnosed in a subject following a period of remission (partial or complete). The tumour may be metastatic or in the same site as the primary tumour.

In some aspects a subject is selected for treatment if they have, are suspected of having, have been diagnosed with, or are at risk of, a circulating tumour or circulating tumour cells (CTC; Gupta et al. 2006, Cell. 127 (4): 679-95; Rack et al., 2014. Journal of the National Cancer Institute. 106 (5)). The CTCs may be, or comprise, metastatic cells (i.e. CTCs capable of establishing metastatic tumours in a subject).

Subjects suspected of having, have been diagnosed with, or are at risk of, a circulating tumour or circulating tumour cells may include;
(1) subjects who have, are suspected of having, have been diagnosed with a primary tumour with metastatic characteristics, such as high-metastatic prognosis or elevated expression of one or more biomarkers of metastatic cancer (Dawood, S., Expert Rev Mol Diagn. 2010 July; 10(5):581-90);
(2) subjects who have, are suspected of having, have been diagnosed with one or more metastatic tumours;
(3) pre-operative or post-operative subjects, wherein the operation is to remove part or all of a solid tumour. Typically, selected pre-operative or post-operative subjects start their treatment not more than 4 weeks from the operation date, such as not more than 2 weeks, or not more than 1 week.

In some cases, a ‘metastatic tumour’ as described herein may be a tumour not located in the same site as the primary tumour.

Samples

The sample may comprise or may be derived from: a quantity of blood; a quantity of serum derived from the subject's blood which may comprise the fluid portion of the blood obtained after removal of the fibrin clot and blood cells; a quantity of pancreatic juice; a tissue sample or biopsy, in particular from a solid tumour; or cells isolated from said subject.

A sample may be taken from any tissue or bodily fluid. In certain aspects, the sample may include or may be derived from a tissue sample, biopsy, resection or isolated cells from said subject.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of CD25 polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In certain aspects, the sample is a fresh biopsy.

Subject Status

The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject may be any of its forms of development, for example, a foetus. In one preferred embodiment, the subject is a human. The terms “subject”, “patient” and “individual” are used interchangeably herein.

In some aspects the subject has, is suspected of having, has been diagnosed with, or is at risk of, a disorder characterized by a disorder-associated antigen (DAA) as described herein. In particular, the disorder characterized by a disorder-associated antigen (DAA) may be solid tumour as described herein.

Tumour Status

In some aspects the subject has an established tumour as defined herein. In some aspects the subject is suspected of having, has been diagnosed with, or is at risk of, a circulating tumour or circulating tumour cells. In some aspects the subject has a metastatic tumour as defined herein.

Controls

In some aspects, target expression in the individual is compared to target expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from individuals who are known to benefit from the treatment. The tissue may be of the same type as the sample being tested. For example, a sample of tumor tissue from a individual may be compared to a control sample of tumor tissue from a individual who is known to be suitable for the treatment, such as a individual who has previously responded to the treatment.

In some cases the control may be a sample obtained from the same individual as the test sample, but from a tissue known to be healthy. Thus, a sample of cancerous tissue from a individual may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of CD25 expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The ADC may comprise an anti-CD25 antibody. The anti-CD25 antibody may be HuMax-TAC™. The ADC may comprise a drug which is a PBD dimer. The ADC may be a anti-CD25-ADC, and in particular, ADCX25/ADCT-301/Camidanlumab Tesirine. The ADC may be an ADC disclosed in WO2014/057119.

The treatment may involve administration of the ADC alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics); and surgery.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentaazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chlorambucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredepa; ethylenimines and methylamelamines including altretamine, triethylenemelamine, triethylenephosphoramide, triethylenethiophosphoramide and trimethylmelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophosphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H—Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure, including vaccine compositions, are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage

It will be appreciated by one of skill in the art that appropriate dosages of the anti-CD25 ADC, and compositions comprising this active element, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of anti-CD25 ADC is determined by the expression of CD25 observed in a sample obtained from the subject. Thus, the level or localisation of expression of CD25 in the sample may be indicative that a higher or lower dose of anti-CD25 ADC is required. For example, a high expression level of CD25 may indicate that a higher dose of anti-CD25 ADC would be suitable. In some cases, a high expression level of CD25 may indicate the need for administration of another agent in addition to the anti-CD25 ADC. For example, administration of the anti-CD25 ADC in conjunction with a chemotherapeutic agent. A high expression level of CD25 may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the anti-CD25 ADC, where it is a PBD bearing ADC, the dosage amounts described above may apply to the conjugate (including the PBD moiety and the linker to the antibody) or to the effective amount of PBD compound provided, for example the amount of compound that is releasable after cleavage of the linker.

The anti-CD25 ADC comprises an anti-CD25 antibody. The anti-CD25 antibody may be HuMax-TAC™. The ADC may comprise a drug which is a PBD dimer. The ADC may be an anti-CD25-ADC, and in particular, ADCX25 or ADCT-301. The ADC may be an ADC disclosed in WO2014/057119.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2 ml, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Anti-CD25 antibodies are known in the art and are useful in the methods disclosed herein. These include antibodies 4C9 (obtainable from Ventana Medical Systems, Inc.). Other suitable antibodies include antibody AB12 described in WO 2004/045512 (Genmab A/S), IL2R.1 (obtainable from Life Technologies, catalogue number MA5-12680) and RFT5 (described in U.S. Pat. No. 6,383,487). Other suitable antibodies include B489 (143-13) (obtainable from Life Technologies, catalogue number MA1-91221), SP176 (obtainable from Novus, catalogue number NBP2-21755), 1B5D12 (obtainable from Novus, catalogue number NBP2-37349), 2R12 (obtainable from Novus, catalogue number NBP2-21755), or BC96 (obtainable from BioLegend, catalogue number V T-072) and M-A251 (obtainable from BioLegend, catalogue number IV A053). Other suitable anti-CD25 antibodies are daclizumab (Zenapax™) and basiliximab (Simulect™), both of which have been approved for clinical use.

### BRIEF DESCRIPTION OF THE FIGURES

Embodiments and experiments illustrating the principles of the disclosure will now be discussed with reference to the accompanying figures in which:

FIG. 1. Sequences

FIG. 2. In vivo MC38 tumour volume following mono-treatment with surrogate ADCx25, anti-PD1 treatment, or control ADC (as per Example 1)

FIG. 3. In vivo MC38 tumour volume showing synergy between low-dose surrogate ADCx25 and anti-PD1 treatment (as per Example 1)

FIG. 4. Re-challenge of tumour-free survivors from MC38 study (per Example 2)

FIG. 5. In vivo CT26 tumour volume following mono-treatment with surrogate ADCx25, anti-PD1 treatment, or control ADC (as per Example 3)

FIG. 6. In vivo CT26 tumour volume showing synergy between low-dose surrogate ADCx25 and anti-PD1 treatment (as per Example 3)

FIG. 7. Re-challenge of tumour-free survivors from CT26 efficacy study (as per Example 4)

FIG. 8. ADCx25 anti-tumour activity is dependent on CD8+ T-cells

FIG. 9. ADCx25 plus PD-1 anti-tumour activity is dependent on CD8+ T-cells

FIG. 10. Spleen T-cell immuno-profiling after SurADCx25 dosing in healthy immuno-competent mice

FIG. 11. Lymph node T-cell immuno-profiling after SurADCx25 dosing in healthy immuno-competent mice

FIG. 12. Blood T-cell immuno-profiling after SurADCx25 dosing in healthy immuno-competent mice

FIG. 13. Thymus T-cell immuno-profiling after SurADCx25 dosing in healthy immuno-competent mice

FIG. 14. Tumour T-cell immuno-profiling after SurADCx25 dosing in CT26 tumor-bearing immuno-competent mice

FIG. 15. Spleen T-cell immuno-profiling after SurADCx25 dosing in CT26 tumor-bearing immuno-competent mice

FIG. 16. Blood T-cell immuno-profiling after SurADCx25 dosing in CT26 tumor-bearing immuno-competent mice

FIG. 17. surADCx25 and radiotherapy combination in vivo study (main)

FIG. 18. surADCx25 and radiotherapy combination in vivo study (rechallenge)

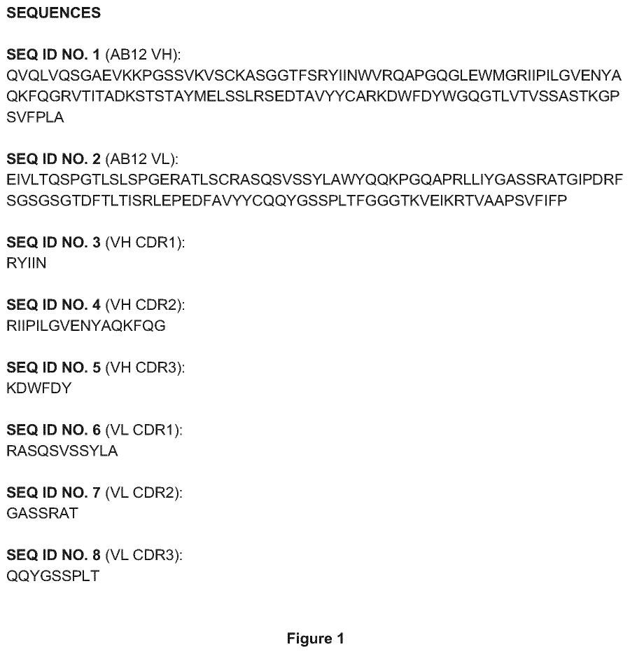

FIG. 19. surADCx25 and radiotherapy combination in vivo study (bilateral)
Each line represents the volume of a single tumour (right OR left flank) from a single animal
The panel title indicates if the panel data relates to tumours from the LEFT (non-irradiated) or RIGHT (irradiated) flank
The panel title also indicates if the animal from which the tumour originated that received: vehicle (control), RadioTx (Radiotherapy only), CD25-ADC (surADCx25 only), or CD25-ADC+RadioTx (combination of radiotherapy & surADCx25)

FIG. 20. surADCx25 and radiotherapy combination in vivo study (sequencing)
Each line represents the volume of a single tumour (right OR left flank) from a single animal
The panel title indicates if the panel data relates to tumours from the LEFT (non-irradiated) or RIGHT (irradiated) flank
The panel title also indicates the order in which the Radiotherapy and surADCx25 were administered:
for “CD25-ADC+RadioTx” the ADC was administered first, on Day 0, with the radiotherapy being administered second, on Day1;
for “RadioTx+CD25-ADC” the radiotherapy was administered first, on Day 0, with the ADC being administered second, on Day1.

FIG. 21. surADCx25 and radiotherapy combination in vivo study (sequencing)
Kaplan-Meier analysis of survival

### STATEMENTS OF INVENTION

1. A method of inducing or enhancing an immune response against a disorder in a subject, the method comprising administering to the subject an effective amount of an anti-CD25 ADC in combination with radiotherapy.
2. The method of statement 1, wherein the immune response is a CD8+ T cell response, a CD4+ T cell response, or an antibody response.
3. The method of any one of statements 1 to 2, wherein the immune response is a CD8+ T cell response.
4. The method of statement 1, wherein the immune response is a memory cell response.
5. A method for treating a disorder in an subject, the method comprising administering to the subject an effective amount of an anti-CD25 ADC in combination with radiotherapy.
6. The method according to statement 5, wherein the method further comprises a step of selecting the subject for treatment and a subject is selected for treatment with the anti-CD25 ADC if:
   (i) the subject has been treated with radiotherapy;
   (ii) the subject is being treated with radiotherapy; and/or
   (iii) the subject is radiosensitive.
7. The method according to any previous statement, wherein the treatment comprises administering the anti-CD25 ADC before the radiotherapy, simultaneous with the radiotherapy, or after the radiotherapy.
8. The method of statement 7, wherein the CD25-ADC and radiotherapy are administered concomitantly.
9. The method of statement 7, wherein the CD25-ADC and radiotherapy are administered on the same day.
10. The method of statement 7, wherein the radiotherapy is administered no longer than 3 weeks before the CD25-ADC.
11. The method of statement 10, wherein the radiotherapy is administered no longer than 1 week before the CD25-ADC.
12. The method of statement 10, wherein the radiotherapy is administered no longer than 1 day before the CD25-ADC.
13. The method of any one of statements 1 to 7, wherein the CD25-ADC is administered before the radiotherapy.
14. The method of statement 13, wherein the CD25-ADC is administered at least 24 hours before the radiotherapy.
15. The method of statement 14, wherein the CD25-ADC is administered at least 2 days before the radiotherapy.
16. The method of any one of statgvfements 13 to 15, wherein the CD25-ADC is administered no longer than 21 days before the radiotherapy.
17. The method of statement 16, wherein the CD25-ADC is administered no longer than 14 days before the radiotherapy.
18. The method of statement 16, wherein the CD25-ADC is administered no longer than 7 days before the radiotherapy.
19. A method of inducing or enhancing an immune response against a disorder in a subject, the method comprising:
   (a) selecting for treatment a subject who has, is, or will be treated with radiotherapy; and
   (b) administering to the subject an effective amount of an anti-CD25 ADC.
20. A method for treating a disorder in a subject, the method comprising:
   (a) selecting for treatment a subject who has, is, or will be treated with radiotherapy; and
   (b) administering to the subject an effective amount of an anti-CD25 ADC.
21. The method of either one of statements 19 or 20, wherein the subjects are selected for treatment by a method comprising (i) identifying a subject who has, is, or will be treated with radiotherapy, then (ii) selecting the subject for treatment if they have, are, or will be treated with radiotherapy.
22. The method of any one of statements 19 to 21, wherein the subject is selected for treatment with the CD25-ADC if they have received, or are expected to receive, radiotherapy on the same day as the ADC administration.
23. The method of any one of statements 19 to 21, wherein the subject is selected for treatment with the CD25-ADC if they have received radiotherapy no longer than 3 weeks before administration of the ADC.
24. The method of statement 23, wherein the subject is selected for treatment with the CD25-ADC if they have received radiotherapy no longer than 1 week before administration of the ADC.
25. The method of statement 24, wherein the subject is selected for treatment with the CD25-ADC if they have received radiotherapy no longer than 1 day before administration of the ADC.
26. The method of any one of statements 19 to 21, wherein the subject is selected for treatment with the CD25-ADC if they are expected to receive radiotherapy at least 24 hours after the ADC administration.
27. The method of statement 26, wherein the subject is selected for treatment with the CD25-ADC if they are expected to receive radiotherapy at least 2 days after the ADC administration.
28. The method of either one of statements 26 or 27, wherein the subject is selected for treatment with the CD25-ADC if they are expected to receive radiotherapy no longer than 21 days after the ADC administration.
29. The method of statements 28, wherein the subject is selected for treatment with the CD25-ADC if they are expected to receive radiotherapy no longer than 14 days after the ADC administration.
30. The method of statements 28, wherein the subject is selected for treatment with the CD25-ADC if they are expected to receive radiotherapy no longer than 7 days after the ADC administration.
31. The method of any one of statements 1 to 8, wherein:
   (i) the immune-suppressive activity of a population of regulatory immune cells in the subject is reduced by at least 90% before the radiotherapy is administered; and/or
   (ii) the size of a population of regulatory immune cells in the subject is reduced by at least 90% before the radiotherapy is administered.
32. The method of statement 9, wherein the regulatory immune cells are Treg cells.
33. The method according to any preceding statement, wherein the subject has a disorder or has been determined to have a disorder.
34. The method according to statement 11, wherein the subject has, or has been has been determined to have, a cancer which expresses CD25 or CD25+ tumour-associated non-tumour cells, such as CD25+ infiltrating cells.
34. The method according to any previous statement, wherein the subject is radiosensitive.
35. The method according to any previous statement, wherein the radiotherapy is focal radiotherapy.
36. The method according to any previous statement wherein the radiotherapy is tumour targeted.
37. The method of any one of statements 1 to 37, wherein the radiotherapy is selected from the group consisting of: external beam radiotherapy, stereotactic radiation therapy, Intensity-Modulated Radiation Therapy, particle therapy, brachytherapy, delivery of radioisotopes, intraoperative radiotherapy, Auger therapy, Volumetric modulated arc therapy, Virtual simulation, 3-dimensional conformal radiation therapy, and intensity-modulated radiation therapy.
38. The method of any one of statements 1 to 38, wherein the radiotherapy is optimized to minimize immunosuppressive effects on immune cells and/or maximise the cytotoxic effect on the targeted tissue.
39. The method of any one of statements 1 to 38, wherein the radiotherapy is sub-therapeutic dose for treatment of the disorder with radiotherapy alone.
40. The method of any one of statements 1 to 38, wherein the total radiotherapy dose is no greater than 40 Gy.
41. The method of any one of statements 1 to 38, wherein the total radiotherapy dose is no greater than 30 Gy.
42. The method of any one of statements 1 to 38, wherein the total radiotherapy dose is no greater than 24 Gy.
43. The method of any one of statements 1 to 38, wherein the total radiotherapy dose is no greater than 20 Gy.
44. The method of any one of statements 1 to 38, wherein the total radiotherapy dose is no greater than 18 Gy.
45. The method of any one of statements 1 to 38, wherein the total radiotherapy dose is no greater than 16 Gy.
46. The method of any one of statements 1 to 38, wherein the total radiotherapy dose is no greater than 15 Gy.
47. The method of any one of statements 1 to 38, wherein the total radiotherapy dose is no greater than 12 Gy.
48. The method of any one of statements 1 to 38, wherein the total radiotherapy dose is no greater than 10 Gy.
49. The method of any one of statements 1 to 48, wherein the radiotherapy is administered as a single dose.
50. The method of any one of statements 1 to 48, wherein the radiotherapy is administered as fractionated doses.
51. The method of statement 50, wherein each fractionated dose is, or is no greater than, 20 Gy.
52. The method of statement 50, wherein each fractionated dose is, or is no greater than, 15 Gy.
53. The method of statement 50, wherein each fractionated dose is, or is no greater than, 12 Gy.
54. The method of statement 50, wherein each fractionated dose is, or is no greater than, 10 Gy.
55. The method of statement 50, wherein each fractionated dose is, or is no greater than, 8 Gy.
56. The method of statement 50, wherein each fractionated dose is, or is no greater than, 6 Gy.
57. The method of statement 50, wherein each fractionated dose is, or is no greater than, 5 Gy.
58. The method of statement 50, wherein each fractionated dose is, or is no greater than, 4 Gy.
59. The method of statement 50, wherein each fractionated dose is, or is no greater than, 3 Gy.
60. The method of statement 50, wherein each fractionated dose is, or is no greater than, 2 Gy.
61. The method of any one of statements 50 to 60, wherein the radiotherapy is administered in two fractionated doses.
62. The method of any one of statements 50 to 60, wherein the radiotherapy is administered in three fractionated doses.
63. The method of any one of statements 50 to 60, wherein the radiotherapy is administered in four fractionated doses.
64. The method of any one of statements 50 to 60, wherein the radiotherapy is administered in five fractionated doses.
65 The method of any one of statements 50 to 60, wherein the radiotherapy is administered in six fractionated doses.
66. The method of any one of statements 50 to 60, wherein the radiotherapy is administered in eight fractionated doses.
67. The method of any one of statements 50 to 60, wherein the radiotherapy is administered in ten fractionated doses.
68. The method of any one of statements 50 to 67, wherein a fractionated dose is administered once daily (QD).
69. The method of any one of statements 50 to 67, wherein a fractionated dose is administered once every other day (Q2D).
70. The method of any one of statements 50 to 67, wherein a fractionated dose is administered once every third day (Q3D).
71. The method of any one of statements 50 to 67, wherein a fractionated dose is administered once weekly (QW).
72. The method according to any previous statement, wherein the disorder is a proliferative disease.
73. The method of statement 72, wherein the proliferative disorder is cancer.
74. The method of either one of statements 72 or 73, wherein the proliferative disorder or cancer is, or is characterized by, one or more solid tumours.
75. The method of statement 74, wherein the treatment induces or enhances an immune response against a solid tumour.
76. The method of either one of statements 74 or 75, wherein the solid tumour is remote from the radiotherapy administration site.
77. The method of any one of statements 74 to 76, wherein the solid tumour is an established tumour.
78. The method of statement 77, wherein the established tumour is a tumour diagnosed or identified in a naïve subject.
79. The method of statement 77, wherein the established tumour is a relapsed tumour.
80. The method of any one of statements 74 to 79, wherein the solid tumour is a metastatic tumour.
81. The method of any one of statements 74 to 80, wherein the solid tumour comprises or consists of CD25−ve neoplastic cells.
82. The method of any one of statements 74 to 81, wherein the solid tumour is associated with CD25+ve infiltrating cells;
   optionally wherein the solid tumour is associated with high levels of CD25+ve infiltrating cells.
83. The method of any one of statements 74 to 82, wherein the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.
84. The method any one of statements 74 to 81, wherein the solid tumour is associated with low levels of CD25+ve infiltrating cells.
85. The method of any one of statements 74 to 81, wherein the solid tumour is not associated with CD25+ve infiltrating cells.
86. The method of either one of statements 72 or 73, wherein the proliferative disorder or cancer is lymphoma or leukaemia.
87. The method of statement 86, wherein the proliferative disorder or cancer is selected from:
   Hodgkin's Lymphoma;
   non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and
   leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).
88. The method of any one of statements 72 to 82, 86 or 87, wherein the proliferative disorder or cancer is associated with elevated levels of regulatory immune cells, such as Treg cells.
89. The method of any one of statements 1 to 88, wherein the CD25-ADC is administered in combination with a checkpoint inhibitor or other immunostimulatory agent.
90. The method of statement 89, wherein the CD25-ADC may be administered before the checkpoint inhibitor or other immunostimulatory agent, simultaneous with the checkpoint inhibitor or other immunostimulatory agent, or after the checkpoint inhibitor or other immunostimulatory agent.
91. The method of either one of statements 89 or 90, wherein the checkpoint inhibitor is a PD1 antagonist.
92. The method of statement 91, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tislelizumab), and BGB-108.
93. The method of either one of statements 89 or 90, wherein the checkpoint inhibitor is a PD-L1 antagonist.
94. The method of statement 93, wherein the PD-L1 antagonist is selected from atezolizumab (Tecentriq), BMS-936559/MDX-1105, durvalumab/MEDI4736, and MSB0010718C (Avelumab).
95. The method of either one of statements 89 or 90, wherein the checkpoint inhibitor is a GITR (Glucocorticoid-Induced TNFR-Related protein) agonist.
96. The method of statement 95, wherein the GITR (Glucocorticoid-Induced TNFR-Related protein) agonist is selected from MEDI1873, TRX518, GWN323, MK-1248, MK 4166, BMS-986156 and INCAGN1876.
97. The method of either one of statements 89 or 90, wherein the checkpoint inhibitor is an OX40 agonist.
98. The method of statement 97, wherein the OX40 agonist is selected from MEDI0562, MEDI6383, MOXR0916, RG7888, OX40mAb24, INCAGN1949, GSK3174998, and PF-04518600.
99. The method of either one of statements 89 or 90, wherein the checkpoint inhibitor is a CTLA-4 antagonist.
100. The method of statement 99, wherein the CTLA-4 antagonist is selected from ipilimumab and Tremelimumab.
101. The method according to any previous statement, wherein the treatment further comprises administering a chemotherapeutic agent.
102 The method of any one of statements 1 to 101, wherein the CD25-ADC comprises a PBD drug moiety, optionally wherein the CD25-ADC is as defined herein in statements 1-110 of the section herein entitled “CD25-ADCs”.
103. The method of any one of statements 1 to 102, wherein the CD25-ADC is ADCx25.
104. The method of any one of statements 1 to 102, wherein the CD25-ADC is ADCT-301.
105. The method of any one of statements 1 to 102, wherein the CD25-ADC is Camidanlumab Tesirine.
106. The method according to any previous statement, wherein the subject is human.
107. An antibody-drug conjugate compound as defined in any one of statements 1 to 106 for use in a method of any one of statements 1 to 106.
108. A composition or pharmaceutical composition comprising an antibody-drug conjugate compound as defined in any one of statements 1 to 106 for use in a method of any one of statements 1 to 106.
109. Use of an antibody-drug conjugate compound as defined in any one of statements 1 to 106 in the preparation of a medicament for use in a method of any one of statements 1 to 106.

### EXAMPLES

### Example 1

In vivo efficacy study of surrogate-ADCx25 in an immuno-competent syngeneic mouse model using mouse colon cancer MC38 cells.

### INTRODUCTION

MC38 is a CD25−ve mouse colon cancer-derived model used pre-clinically in immunotherapy-type studies which is known to have infiltration of Treg and Teff cells.

In Arce Vargas et al., 2017, Immunity 46, 1-10, Apr. 18, 2017 (http://dx.doi.org/10.1016/j.immuni.2017.03.013) selective depletion of tumor infiltrating Treg cells in the MC38 model was shown using an Fc enhanced version of PC61, a rat antibody directed against mouse CD25 and synergy with PD1 was described. The wild-type PC61 was conjugated to the PBD dimer drug-linker SG3249 (the PBD drug-linker used in ADCx25/ADCT-301/Camidanlumab Tesirine) and designated as Surrogate-ADCx25 (or SurADCx25). The efficacy of Surrogate-ADCx25 was studied as monotherapy or in combination with anti-PD1 (Anti-PD1, clone RPM1-14, BioXcell cat #BE0146) in the MC38 syngeneic mouse model.

Study Design

Female C57BL/6 mice (C57BL/6NCrl, Charles River) were nine weeks old on Day 1 of the study and had a body weight (BW) range of 17.8 to 24.2 g. At the completion of the initial study described in this example, tumor-free survivors were transferred to a secondary rechallenge study, described in Example 2.

On the day of implant 5×10⁵ MC38 cells (0.1 mL suspension) were subcutaneously implanted into the right flank of each test animal. Tumors were monitored as their volumes approached the target range of 80-120 mm³. Fifteen days after tumor cell implantation, on Day 1 of the study, animals were sorted into ten groups (n=10/group) with individual tumor volumes of 63 to 172 mm3, and group mean tumor volumes of 103-172 mm3.

All doses were administered intraperitoneally (i.p.) on Day 1 except for anti-PD-1 which was administered once on Days 2, 5, 8. The dosing volume was 0.2 mL per 20 grams of body weight (10 mL/kg), and was scaled to the body weight of each individual animal. Tumors were measured twice per week until the study was ended on Day 59. Each animal was euthanized when its tumor attained the endpoint tumor volume of 1000 mm3 or on the final day, whichever came first.

Surrogate-ADCx25 was administered intraperitoneally (i.p.) as single dose (0.1, 0.5 and 1 mg/kg) on day 1 either alone or in combination with anti-PD1 antibody (given at standard dosing regime, i.e. 5 mg/kg at day 2, 5 and 8). As a control, Isotype Control ADC (B12-SG3249) was administered as single dose (1 mg/kg) on day 1 either alone or in combination with anti-PD1 antibody (given at standard dosing regime), while anti-PD1 antibody was administered alone at standard dosing regimen.

Tumors were measured in two dimensions using calipers, and volume was calculated using the formula:

Tumor Volume (mm³)=w²×l/2, where w=width and l=length, in mm, of the tumor.

Tumor weight may be estimated with the assumption that 1 mg is equivalent to 1 mm3 of tumor volume.

Results

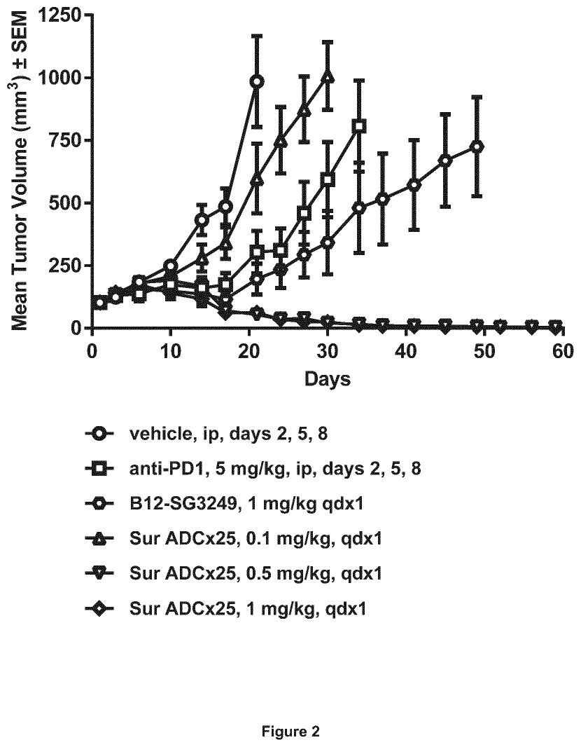

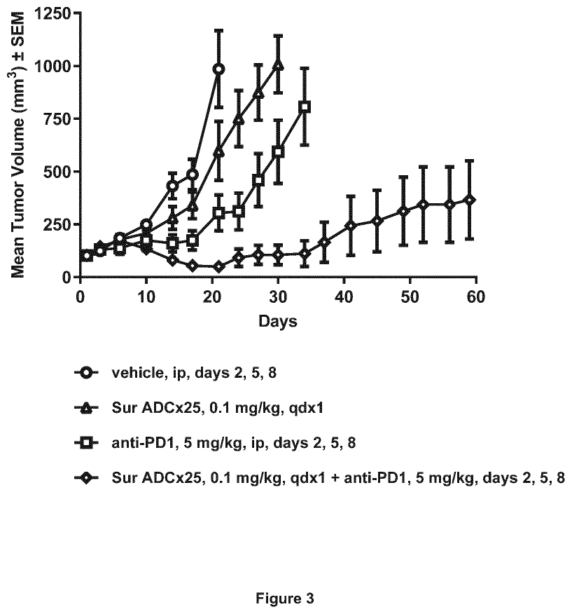

Surrogate-ADCx25 had strong and dose-dependent anti-tumor activity per se in the MC38 syngeneic model. The isotype control ADO had significant lower activity than surrogate-ADCx25 at 1 mg/kg. (FIG. 2). A strong synergy was observed when combining a low single dose of surrogate-ADCx25 with anti-PD1 antibody (FIG. 3). High efficacy of higher doses of surrogate-ADCx25 in the present model prevented assessment of synergy at higher doses.

In vivo, a single dose of sur-ADCx25 at 0.5 or 1 mg/kg induced strong and durable anti-tumor activity against established CD25-negative solid tumors with infiltrating Treg cells (MC38 syngeneic model).

| Response summary | PR | CR | TFS |
|---|---|---|---|
| Vehicle | 0 | 0 | 0 |
| Sur ADCX25, 0.1 mg/kg | 0 | 1 | 1 |
| Sur ADCX25, 0.5 mg/kg | 2 | 8 | 8 |
| Sur ADCX25, 1 mg/kg | 2 | 8 | 8 |
| Anti-PD1, 5 mg/kg | 0 | 3 | 3 |
| B12-SG3249, 1 mg/kg | 2 | 2 | 2 |
| Sur ADCX25, 0.1 mg/kg + | 1 | 6 | 6 |
| anti-PD1 | | | |
| Sur ADCX25, 0.5 mg/kg + | 1 | 9 | 9 |
| anti-PD1 | | | |
| Sur ADCX25, 1 mg/kg + | 0 | 10 | 10 |
| anti-PD1 | | | |
| B12-SG3249, 1 mg/kg + | 2 | 5 | 5 |
| anti-PD1 | | | |

| | |
|---|---|
| | Coefficient of Drug Interaction (CDI) |
| | Sur ADCX25, 0.1 mg/kg; |
| | anti-PD1,5 mg/kg; |
| | Sur ADCX25, 0.1 mg/kg + anti-PD1 |
| Day 21 | 0.268 (Synergism) |

Response Table Criteria (Also Applicable to Example 3 and Example 5):

Treatment may cause partial regression (PR) or complete regression (CR) of the tumor in an animal.

In a PR response, the tumor volume is 50% or less of its Day 1 volume for three consecutive measurements during the course of the study, and equal to or greater than 13.5 mm³ for one or more of these three measurements.

In a CR response, the tumor volume is less than 13.5 mm³ for three consecutive measurements during the study.

Any animal with a CR response at the end of the study was additionally classified as a tumor-free survivor (TFS).

Animals were scored only once during the study for a PR or CR event and only as CR if both PR and CR criteria were satisfied.

CDI Methodology Also Applicable to Example 3 and Example 5):

The Coefficient of Drug Interaction (CDI) (11) in were assessed for subadditive, additive, or supra-additive (synergism) properties on Day 21, the last day all evaluable animals remained on study.

The CDI was determined according to the equation below:

CDI=AB/AxB
Where,
   x=mean tumor volume
   AB=xAB/xC
   A=xA/xC
   B=xB/xC

CDI<1 is supra-additive (i.e. synergism); CDI=1 is additive; CDI>1 is subadditive

### Example 2

Re-Challenge of Tumor-Free Survivors from Example 1 MC38 Efficacy Study

The complete responders from Example 1 and 10 naïve control female C57BL/6 mice were or 17-18 weeks old on Day 1 of this study and had a BW range of 20.9 to 39.0 g.

On Day 1 of the re-challenge study, 5×105 MC38 cells (0.1 mL suspension) were subcutaneously implanted into the left flank (contralateral to the original cell implant) and tumor growth was monitored. No ADC or anti-PD-1 treatment was administered in the rechallenge study.

Animal handling and tumour measurement was as in Example 1 unless otherwise stated.

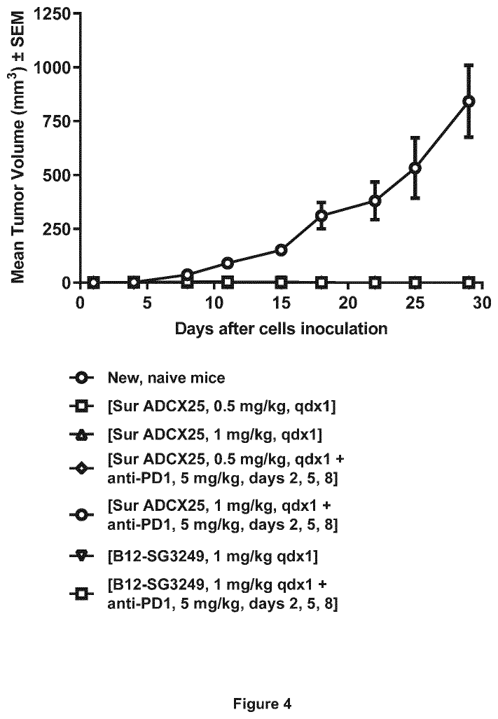

Results: Re-challenged animals did not develop new tumors indicating ADCx25 was able to induce tumor-specific protective immunity (see FIG. 4).

### Example 3

In vivo efficacy study of surrogate-ADCx25 in an immuno-competent syngeneic mouse model using mouse colon cancer CT26 cells

Introduction

CT26 is a CD25−ve mouse colon cancer-derived model used pre-clinically in immunotherapy-type studies which is known to have infiltration of Treg and Teff cells.

Study Design

Female BALB/c mice (BALB/cNCrl, Charles River) were nine weeks old on Day 1 of the study and had a body weight (BW) range of 17.2 to 23.3 g. At the completion of the study, tumor-free survivors were transferred to they re-challenge study described in Example 4.

On the day of implant 3×10⁵ CT26 cells (0.1 mL suspension) were subcutaneously implanted into the right flank of each test animal. Tumors were monitored as their volumes approached the target range of 80-120 mm³. Ten days after tumor cell implantation, on Day 1 of the study, animals were sorted into ten groups (n=10/group) with individual tumor volumes of 75 to 162 mm³, and group mean tumor volumes of 110-111 mm³. All doses were administered intraperitoneally (i.p.) on Day 1 except for anti-PD-1 which was administered once on Days 2, 5, 8. The dosing volume was 0.2 mL per 20 grams of body weight (10 mL/kg), and was scaled to the body weight of each individual animal. Tumors were measured twice per week until the study was ended on Day 48. Each animal was euthanized when its tumor attained the endpoint tumor volume of 2000 mm³ or on the final day, whichever came first.

Tumors were measured in two dimensions using calipers, and volume was calculated using the formula:

Tumor Volume (mm³)=w2×l/2, where w=width and l=length, in mm, of the tumor.

Tumor weight may be estimated with the assumption that 1 mg is equivalent to 1 mm³ of tumor volume.

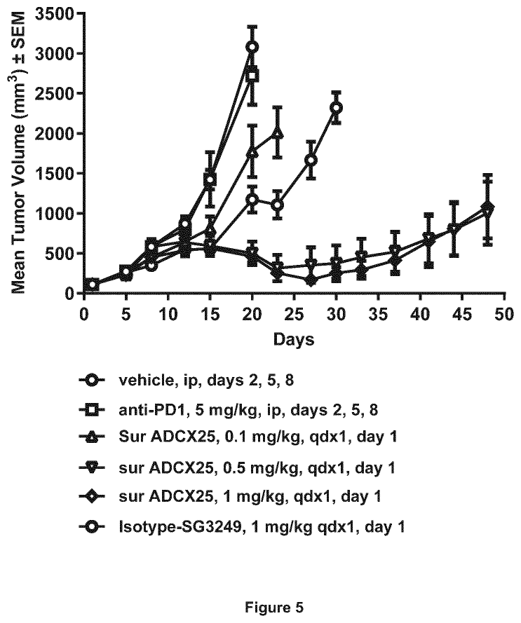

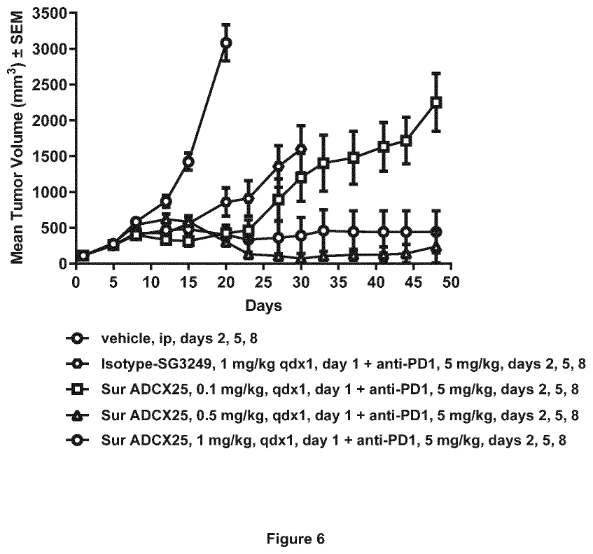

Results: In vivo, a single dose of sur-ADCx25 at 0.5 or 1 mg/kg induced strong and durable anti-tumor activity against established CD25-negative solid tumors with infiltrating Treg cells (CT26 syngeneic model); see FIG. 5 and FIG. 6.

| Response summary | PR | CR | TFS |
|---|---|---|---|
| Vehicle | 0 | 0 | 0 |
| Sur ADCX25, 0.1 mg/kg | 0 | 0 | 0 |
| Sur ADCX25, 0.5 mg/kg | 1 | 2 | 2 |
| Sur ADCX25, 1 mg/kg | 1 | 3 | 3 |
| Anti-PD1, 5 mg/kg | 0 | 0 | 0 |
| Isotype-ADC, 1 mg/kg | 0 | 0 | 0 |
| Sur ADCX25, 0.1 mg/kg + | 1 | 1 | 1 |
| anti-PD1 | | | |
| Sur ADCx25, 0.5 mg/kg + | 0 | 7 | 7 |
| anti-PD1 | | | |
| Sur ADCX25, 1 mg/kg + | 0 | 8 | 8 |
| anti-PD1 | | | |
| Isotype-ADC, 1 mg/kg + | 0 | 1 | 1 |
| anti-PD1 | | | |

| | |
|---|---|
| | Coefficient of Druq Interaction (CDI) |
| | SurADCX25, 0.1 mg/kg; |
| | anti-PD1,5 mg/kg; |
| | Sur ADCX25, 0.1 mg/kg + anti-PD1 |
| Day 20 | 0.285 (Synergism) |

### Example 4

Re-Challenge of Tumor-Free Survivors from Example 3 CT26 Efficacy Study

The complete responders from Example 3 and 10 naïve control female BALB/c mice were or 16-17 weeks old on Day 1 of the study and had a BW range of 18.2 to 24.4 g.

On Day 1 of the re-challenge study 3×10⁵ CT26 cells (0.1 mL suspension) were subcutaneously implanted into the left flank (contralateral to the cell implant of example 3) and tumor growth was monitored and measured as described in example 3.

No treatment was administered in the re-challenge study.

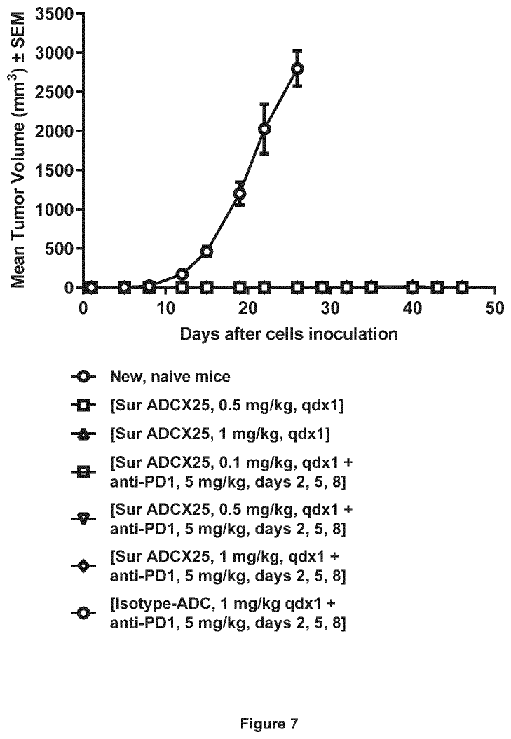

Results: Re-challenged animals did not develop new tumors indicating ADCx25 was able to induce tumor-specific protective immunity (see FIG. 7).

### Example 5

surADCx25 Anti-Tumour Activity Against CD25-Ve Tumours is Dependent on C8+ T-Cells

Study Design

Female C57BL/6 mice (C57BL/6NCrl, Charles River) were eleven weeks old on Day 1 of the study and had a body weight (BW) range of 18.6 to 28.2 g.

On the day of implant 5×10⁵ MC38 cells (0.1 mL suspension) were subcutaneously implanted into the right flank of each test animal. Tumors were monitored as their volumes approached the target range of 80-120 mm³.

Fifteen days after tumor cell implantation, on Day 0 of the study, animals were sorted into groups (n=10/group) with individual tumor volumes of 75 to 126 mm3, and with group mean tumor volumes of 86-89 mm³. All doses were administered intraperitoneally (i.p.).

Anti-CD8-2.43 murine monoclonal antibody was administered once daily on Days 0, 5, 8 and 13 in order to deplete and suppress levels of CD8+ T-cells.

SurADCx25 dose was administered on Day 1.

Anti-PD-1 was administered once daily on Days 2, 5, 8.

The dosing volume was 0.2 mL per 20 grams of body weight (10 mL/kg), and was scaled to the body weight of each individual animal.

Tumors were measured twice per week until the study was ended (day 29 for anti-CD8+surADCx25 and anti-CD8+anti-PD1+surADCx25 groups; day 44 for surADCx25 alone and surADCx25+anti-CD8).

Each animal was euthanized when its tumor attained the endpoint tumor volume of 1000 mm³ or on the final day, whichever came first.

Results

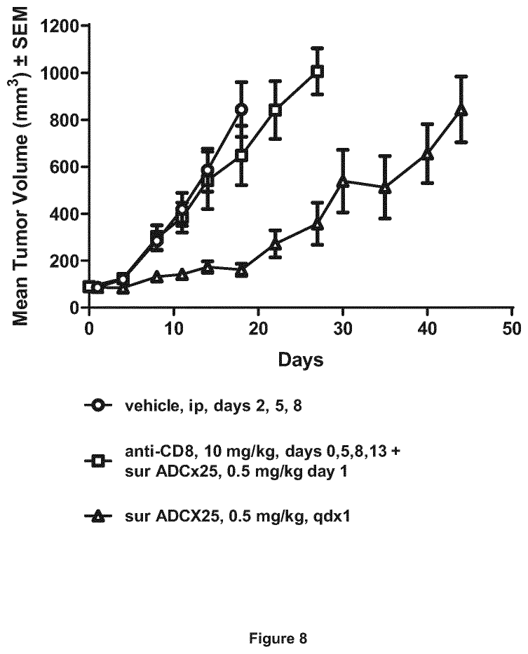

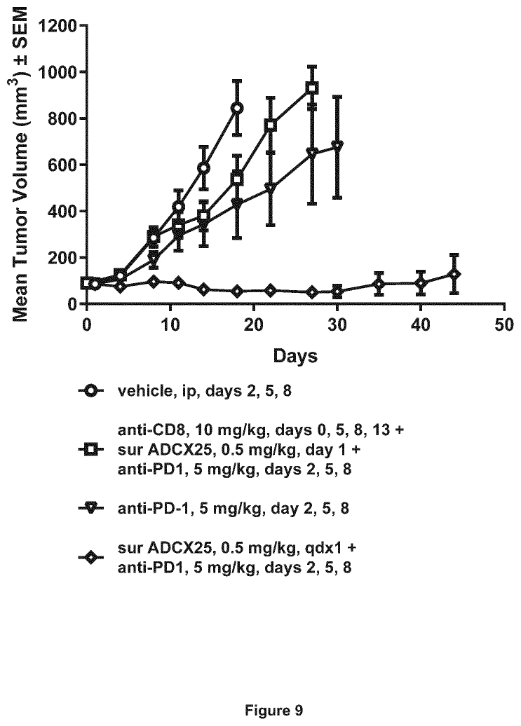

Sur-ADCx25 anti-tumor activity, either alone (FIG. 8) or combined with an anti-PD1 antibody (FIG. 9), was significantly reduced when CD8+ T-cells are depleted, indicating that surADCx25 activity is CD8+ T-cell-dependent and that overall effector T-cell responses were not negatively impacted by sur-ADCx25.

| Response summary | PR | CR | TFS |
|---|---|---|---|
| Vehicle | 0 | 0 | 0 |
| Anti-PD1 | 2 | 0 | 0 |
| Sur ADCX25 | 0 | 1 | 1 |
| Sur ADCX25 + anti-CD8 | 0 | 0 | 0 |
| Sur ADCX25 + anti-PD1 | 2 | 5 | 5 |
| Sur ADCX25 + anti-PD1 + | 0 | 0 | 0 |
| anti-CD8 | | | |

| | |
|---|---|
| | Coefficient of Drug Interaction (CDI) |
| | Sur ADCX25, 0.5 mg/kg; |
| | anti-PD1,5 mg/kg; |
| | Sur ADCX25, 0.5 mg/kg + anti-PD1 |
| Day 22 | 0.471 (Synergism) |

### Example 6

T-Cell Immuno-Profiling after SurADCx25 Dosing in Healthy Immuno-Competent Mice

Study Design

Eight to twelve week old, female C57BL/6 mice were dosed intravenously on day 1 with either surADCx25 (0.5 mg/kg) or an isotype control ADC (0.5 mg/kg). A non-dosed group acted as control (each group contained 24 mice).

Terminal samples (blood, spleen, lymph node and thymus) were obtained on days 1 (4 hrs post-dose), 7, 14 and 21 from 6 animals per group at each time-point. Additional non-terminal blood samples (mandibular bleeds) were obtained on days 4, 11 and 18 from 6 animals per group at each time-point.

Samples (tissue and blood) were processed for flow cytometry assessment and CD4+ T-cells (CD45⁺ CD3⁺ CD4⁺ CD8⁻), CD8⁺ T-cells (CD45⁺ CD3⁺ CD4⁻ CD8⁺) and T_{reg} cell (CD45⁺ CD3⁺ CD4⁺ CD25⁺ FoxP3⁺) content determined. Data represent the mean±SEM of T_{reg} cell population in the assayed tissue as a percentage of CD45⁺.

Results

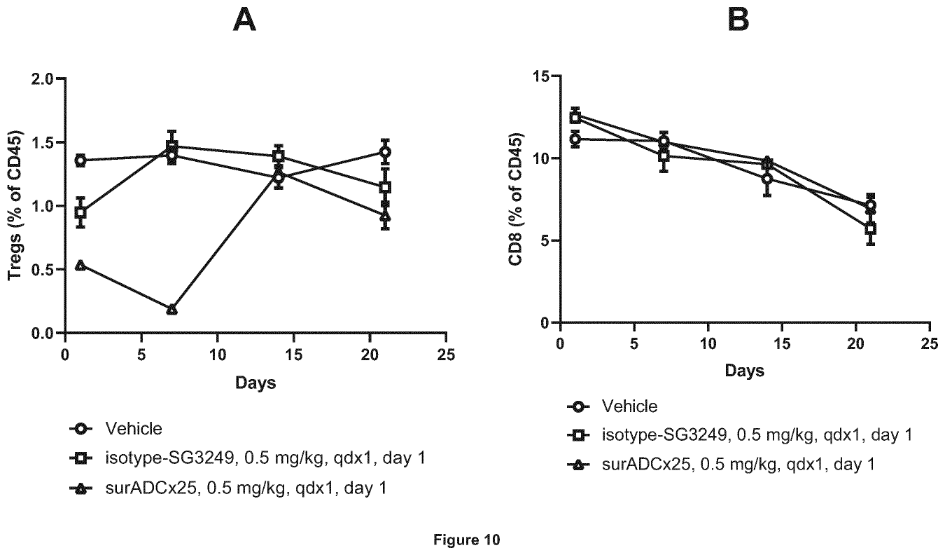

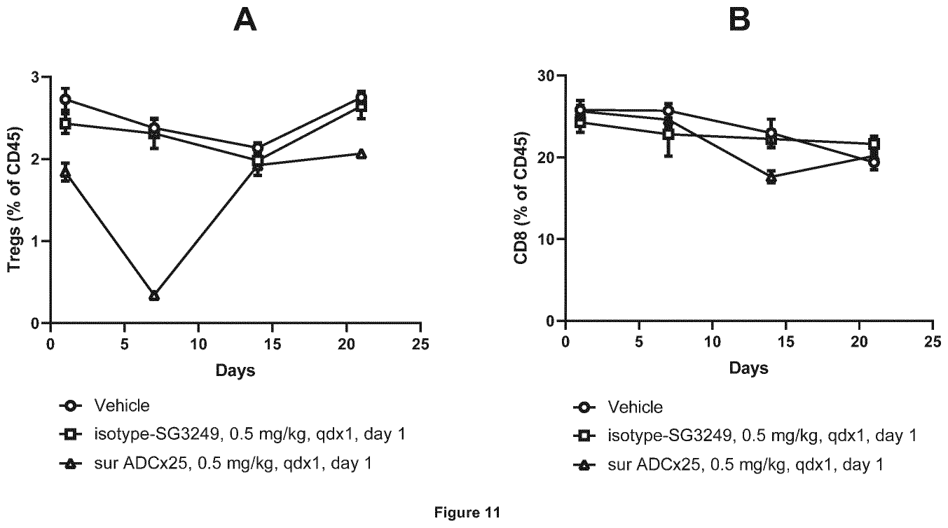

Spleen:
→ a clear depletion of spleen Tregs was observed at 1 day & 7 days post surADCx25 administration, with Treg levels mostly recovering by day 14 (see FIG. 10A; % shown indicates the % reduction compared to vehicle)
→ there was no observed impact on the level of spleen CD8+ Teff cells (see FIG. 10B)

Lymph Nodes:
→ a clear depletion of lymph node Tregs was observed at 1 day & 7 days post surADCx25 administration, with Treg levels mostly recovering by day 14 (see FIG. 11A; % shown indicates the % reduction compared to vehicle)
→ there was no observed impact on the level of lymph node CD8+ Teff cells (see FIG. 11B)

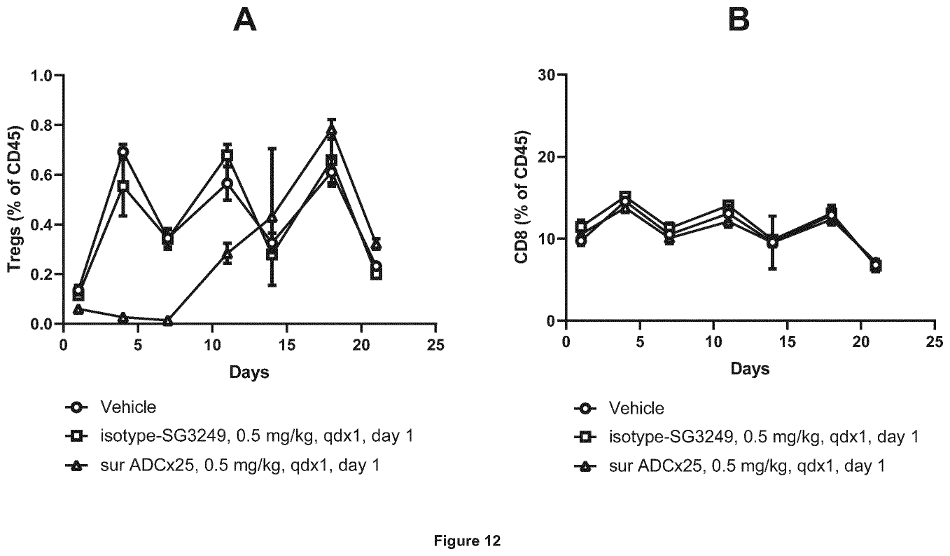

Blood:
→ Increased variability in vehicle and isotype-control values due to low levels of events; measured with additional non-terminal blood samples (days 4, 11 and 18).
→ a clear depletion of blood Tregs was observed at 1 day, 7 days, and 11 days post surADCx25 administration, with Treg levels recovering by day 14 (see FIG. 12A; % shown indicates the % reduction compared to vehicle)
→ there was no observed impact on the level of blood CD8+ Teff cells (see FIG. 12B)

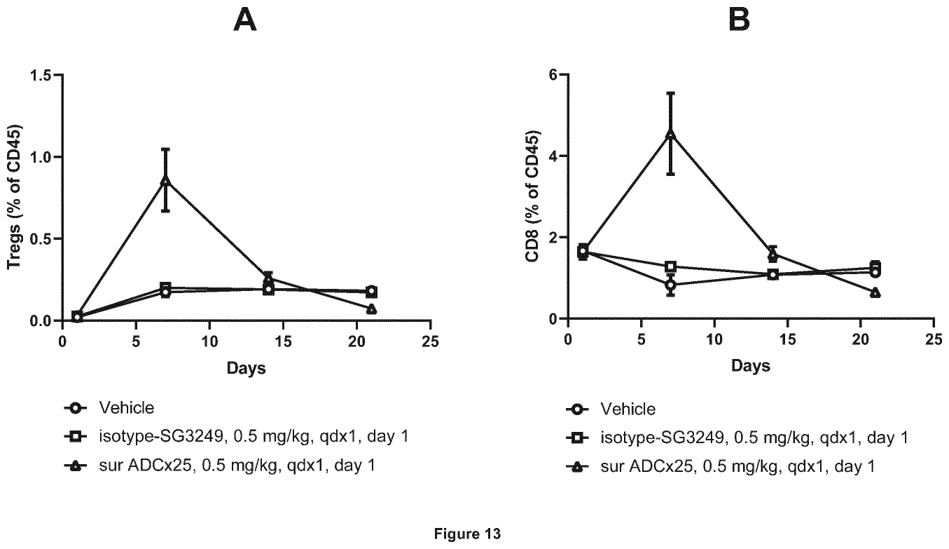

Thymus:
→ a clear increase of thymus Tregs & CD8+ Teffs was observed at 7 days post surADCx25 administration, with Treg & CD8+ Teff levels mostly recovering by day 14 (see FIGS. 13A & B)

### SUMMARY

A single dose of SurADCx25 caused significant depletion of Tregs in spleen, lymph nodes and blood (>95%).

There was a clear increase in the thymus in the amount of Tregs at 7 days post-dose.

There was no depletion of Teff cells from spleen, lymph nodes and blood caused by SurADCx25, however, an increase in thymus Teff cells was also observed 7 days post dosing of SurADCx25.

Around Day 15, Tregs levels in blood, spleen, thymus and lymph nodes are restored to normal (vehicle control).

### Example 7

T-Cells Immuno-Profiling after surADCx25 Dosing in CT26 Tumor-Bearing Immune-Competent Mice

Study Design

Female BALB/c mice were ten weeks old on Day 1 of the study.

Cultured CT26 cells were harvested during log phase growth and resuspended in phosphate buffered saline (PBS) at a concentration of 3×10⁶ cells/mL. Tumors were initiated by subcutaneously implanting 3×10⁵ CT26 cells into the right flank of each test animal. Fourteen days after tumor cell implantation, on Day 1 of the studies, animals were sorted into groups (n=24 or 18) with mean tumor volumes of 115-116 mm³.

SurADCx25 was administered intraperitoneally (i.p.) on Day 1. Anti-PD-1 was administered i.p. once daily on Days 2, 5, 8. Group 1 received the PBS vehicle and served as the control. Group 2 received anti-PD-1 at 5 mg/kg. Groups 3 received surADCx25 at 1 mg/kg. Group 4 received surADCx25 at 1 mg/kg in combination with anti-PD-1 at 5 mg/kg.

Samples were collected for analysis by flow cytometry on Days 1 (pre-dose; Group 1 only, n=6), 3 (Groups 1-4, n=6) and 9 (Groups 1-4, n=6). Full blood volume was collected from each animal via terminal cardiac puncture and was processed for flow cytometry.

Immediately following blood collection, the tumor and the spleen were harvested from each animal and processed for flow cytometry and CD4+ T-cells (CD45⁺ CD3⁺ CD4⁺ CD8⁻), CD8+ T-cells (CD45⁺ CD3⁺ CD4⁻ CD8⁺) and Treg cell (CD45⁺ CD3⁺ CD4⁺ CD25⁺ FoxP3⁺) content determined.

Results

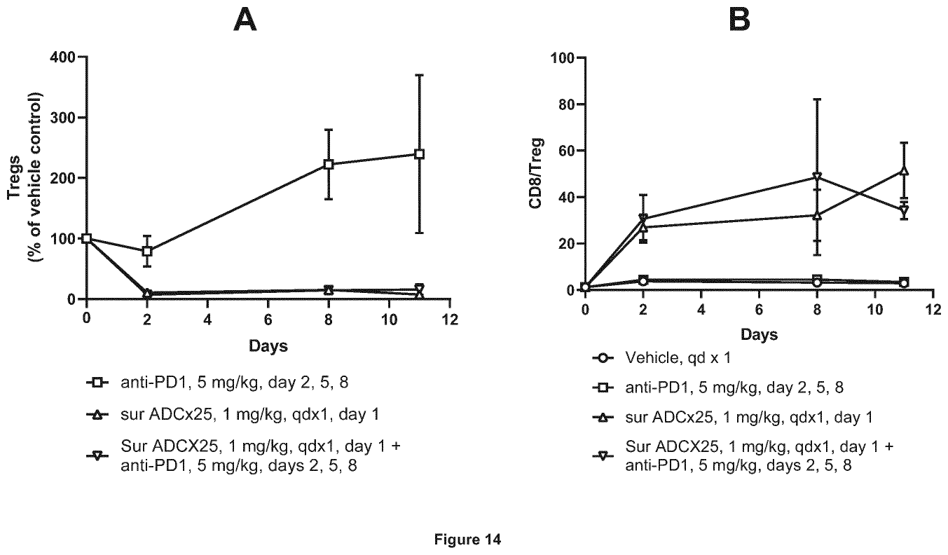

Tumour:
→ a significant and sustained depletion of tumour Tregs was observed from 2 days through 11 days post surADCx25 administration (see FIG. 14A)
→ an increased tumour CD8+ Teff/Tregs ratio was observed from 2 days through 11 days post surADCx25 administration (see FIG. 14B).

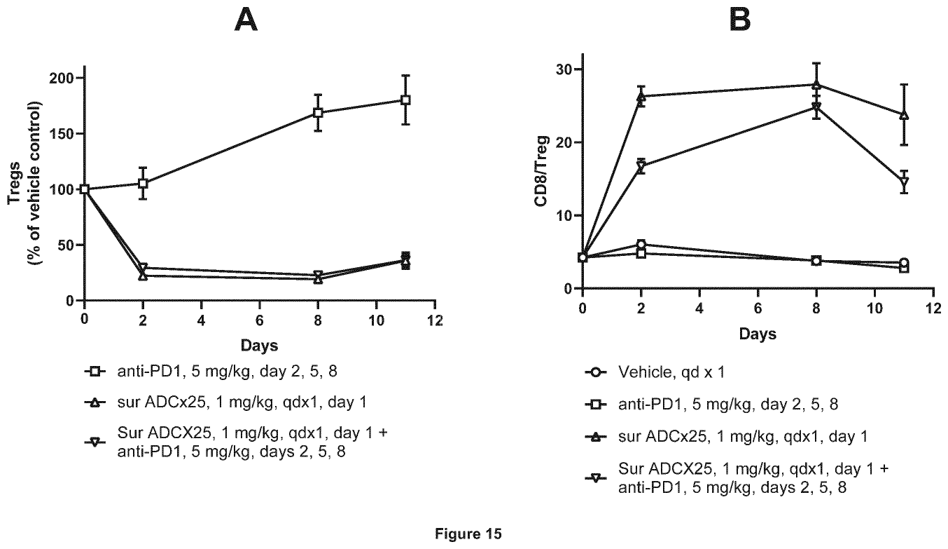

Spleen:
→ a significant and sustained depletion of spleen Tregs was observed from 2 days through 11 days post surADCx25 administration (see FIG. 15A)
→ an increased spleen CD8+ Teff/Tregs ratio was observed from 2 days through 11 days post surADCx25 administration (see FIG. 15B).

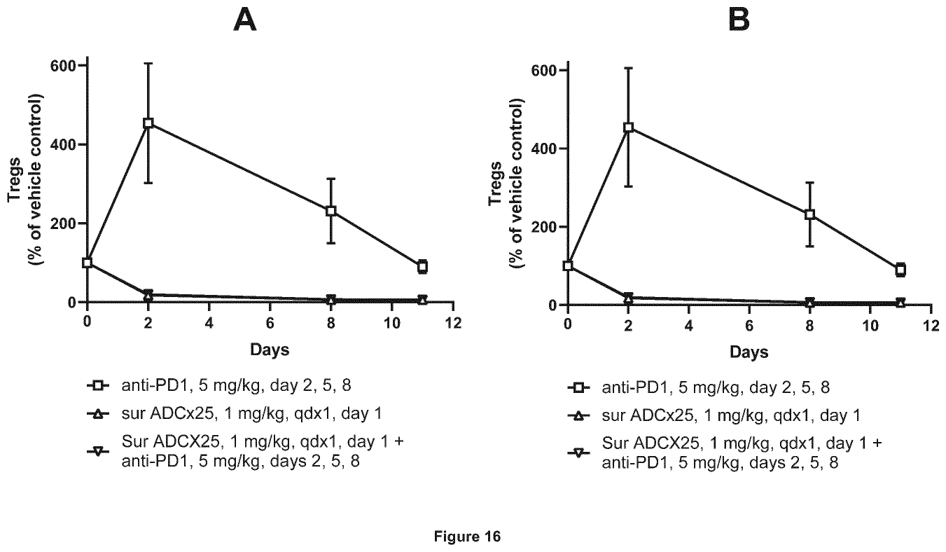

Blood:
→ a significant and sustained depletion of blood Tregs was observed from 2 days through 11 days post surADCx25 administration (see FIG. 16A)
→ an increased blood CD8+ Teff/Tregs ratio was observed from 2 days through 11 days post surADCx25 administration (see FIG. 16B).

Summary and Conclusions

A single dose of surADCx25 to immuno-competent mice bearing established CT26 tumors caused significant and sustained depletion of Tregs in tumors, blood, and spleen.

The simultaneous increase observed in CD8+ Teff/Tregs ratio in tumors, blood, and spleen indicates that surADCx25 did not negatively impact the overall Teff cell response.

Together, the data set out in Examples 5 to 7 indicate that SurADCx25 depletion of Tregs together with activation of the CD8+ Teff response is an important mode of action in surADCx25's anti-tumour activity.

### Example 8

In Vivo Studies of Combined Treatment with surADCx25 and Radiotherapy

Methodology

Female BALB/c mice (BALB/cAnNHsd, Envigo) were 6-7 weeks old (main study) at day of implant and had a body weight (BW) range of 17.8 to 18.7 g.

On the day of implant of the main study, 5×10⁵ CT26 cells (0.2 mL suspension) were subcutaneously implanted into the right high axilla of each test animal. Tumors were monitored as their volumes approached the target range. Seven days after tumor cell implantation, on Day 1 of the study, animals were sorted into six groups (n=10/group) with group mean tumor volumes of 95 mm³. surADCx25 (also described herein as “sur301”) was administered intravenously (i.v.) on Day 1, while tumor-targeted radiation treatment was administered once on Day 2 at 5 Gy. Tumors were measured thrice per week until the study was ended on Day 62. Each animal was euthanized when its tumor attained the endpoint tumor volume of 2000 mm³ or on the final day, whichever came first.

At the completion of the main study, tumor-free survivors were transferred to a secondary rechallenge study. The complete responders and 10 age matched, naïve control female BALB/c mice were used in this re-challenge study.

On Day 1 of the re-challenge study, 5×10⁵ CT26 cells (0.2 mL suspension) were subcutaneously implanted into the left high axilla (contralateral to the original cell implant) and tumor growth was monitored. No treatment was administered in the re-challenge study.

In both the main and re-challenge studies, tumors were measured in two dimensions using calipers, and volume was calculated using the formula:

Tumor Volume (mm³)=w²×l/2, where w=width and l=length, in mm, of the tumor.

Tumor weight may be estimated with the assumption that 1 mg is equivalent to 1 mm³ of tumor volume.

Results: Main Study

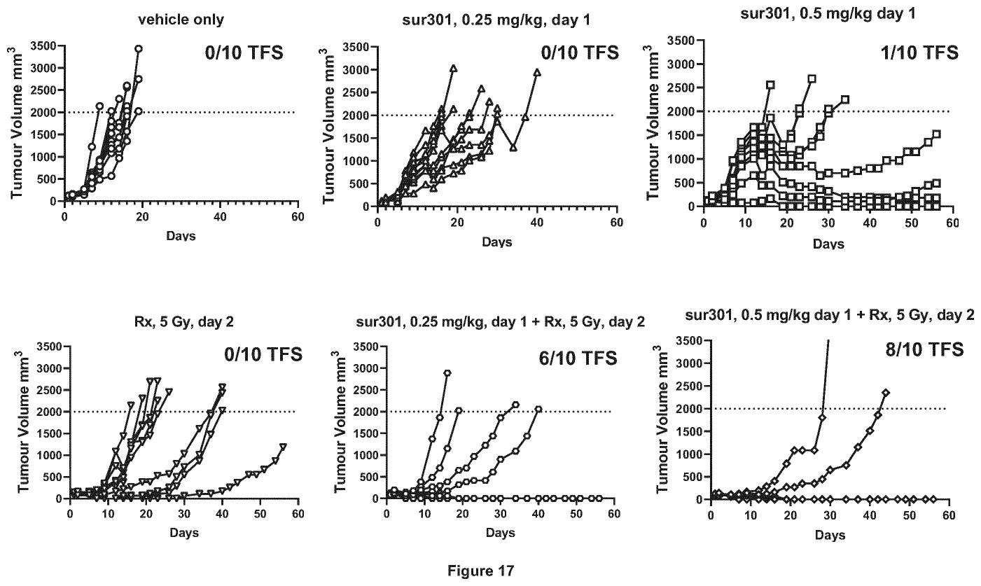

See FIG. 17 for full results.

| Response summary | PR | CR | TFS |
|---|---|---|---|
| Vehicle | 0 | 0 | 0 |
| Sur301, 0.25 mg/kg | 0 | 0 | 0 |
| Sur301, 0.5 mg/kg | 0 | 1 | 1 |
| Radiotherapy (Rx), 5 Gy | 0 | 3 | 0 |
| Sur301 ,0.25 mg/kg + Rx, 5 Gy | 0 | 6 | 6 |
| Sur301, 0.5 mg/kg + Rx, 5 Gy | 0 | 8 | 8 |

| | |
|---|---|
| | Coefficient of Drug Interaction (CDI) |
| | Sur301, 0.25 mg/kg; |
| | Rx, 5 Gy; |
| | Sur301, 0.25 mg/kg + Rx, 5 Gy |
| Day 15 | 0.88 (Synergism) |

| | |
|---|---|
| | Coefficient of Drug Interaction (CDI) |
| | Sur301,0.5 mg/kg; |
| | Rx, 5 Gy; |
| | Sur301, 0.5 mg/kg + Rx, 5 Gy |
| Day 15 | 0.47 (Synergism) |

[CDI<1 indicates a synergistic effect; CDI=1 indicates an additive effect; CDI>1 indicates an antagonistic effect.]

Results: Re-Challenge Study

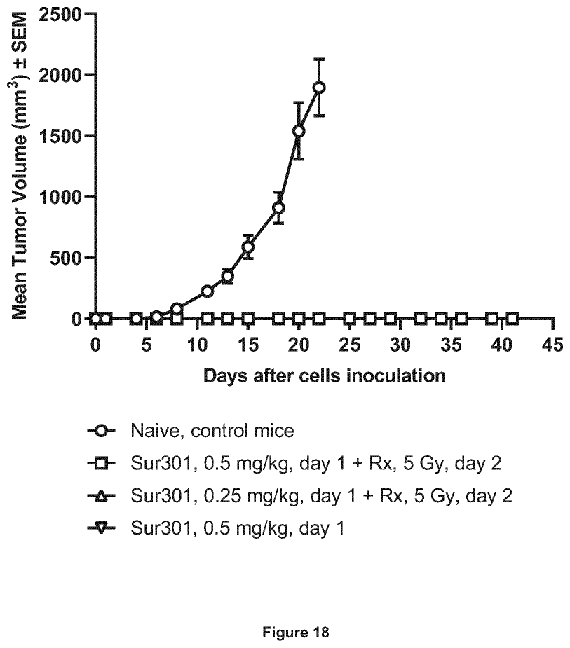

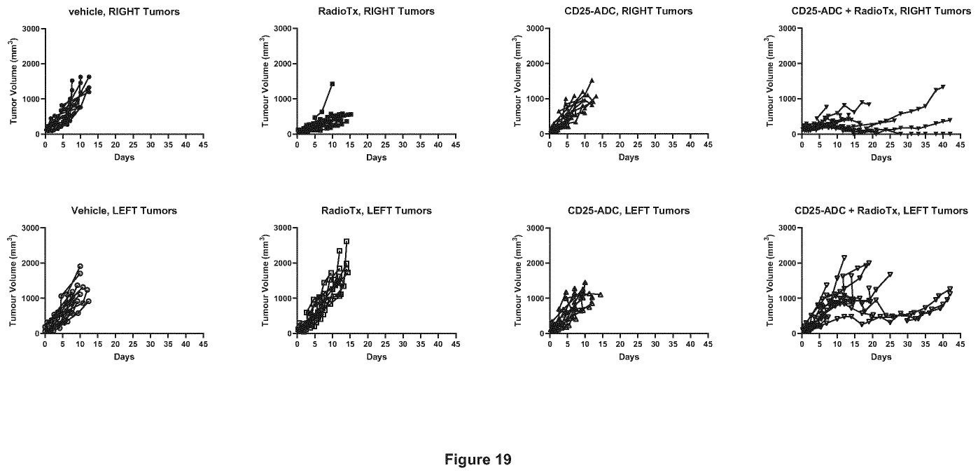

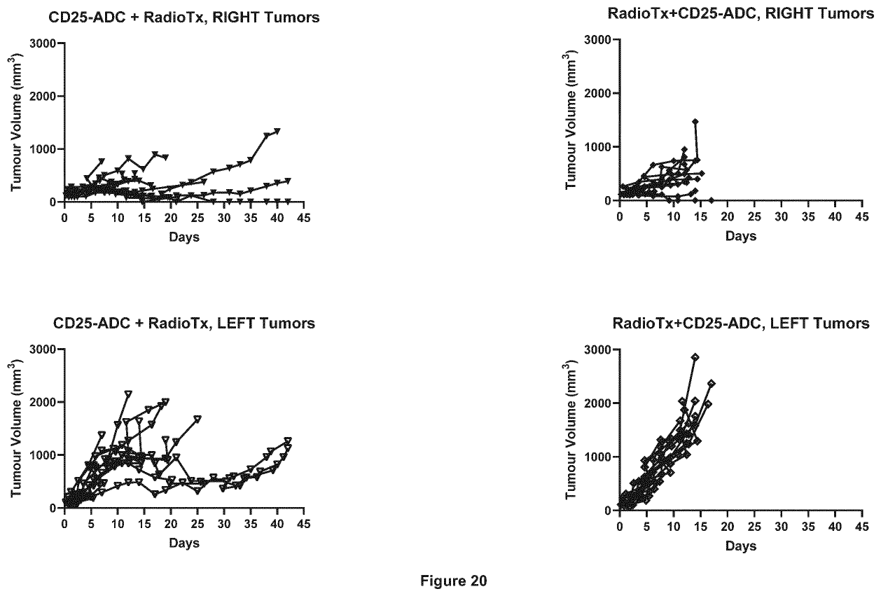

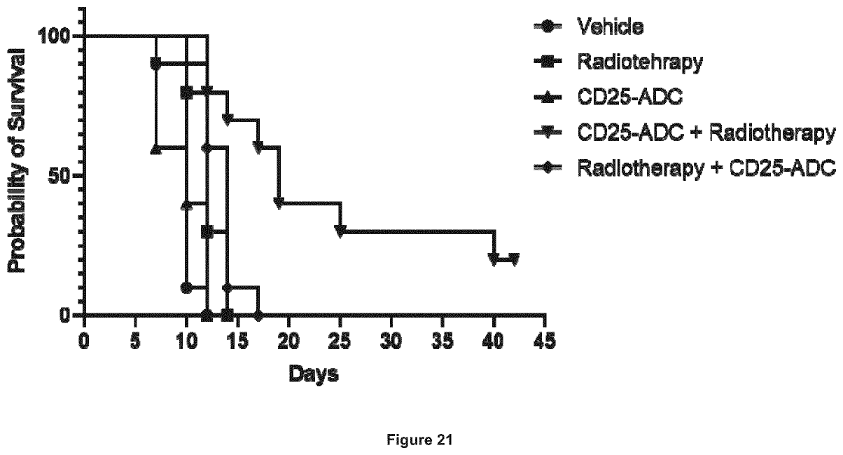

See FIG. 18 for full results.

Conclusions

Synergy between sur301 and radiotherapy is observed at both of the analysed dose levels.

In the re-challenge study, no tumour growth was observed on re-inoculation of Tumour Free Survivors from either the groups treated with the sur301+Rx combination (14 individuals in total), or the sole TFS from the sur301, 0.5 mg/kg group. This is consistent with an immune-based therapeutic mechanism such as the abscopal effect.

### Example 9

In Vivo Studies of Combined Treatment with surADCx25 and Radiotherapy; Bilateral Testing for an Abscopal Effect

Methodology

Female BALB/c mice (BALB/cAnNHsd, Envigo) were 7-8 weeks old at day of implant.

On the day of implant, 5×10⁵ CT26 cells (0.2 mL suspension) were subcutaneously implanted into the right and left high axilla of each test animal. Tumors were monitored as their volumes approached the target range. Ten days after tumor cell implantation, on Day 0 of the study, animals were sorted into groups (n=10/group) with group mean tumor volumes of 105 mm³ (right tumors) and 99 mm³ (left tumors).

Two treatment schedules were tested:
1) CD25-ADC was administered intravenously (i.v.) on Day 0, and tumor-targeted radiation was administered once to the tumors on the right flanks on Day 1 at 5 Gy. Tumours on the left flank were not irradiated.
2) Tumor-targeted radiation was administered once to the tumors on the right flanks on Day 0 at 5 Gy and CD25-ADC was administered intravenously (i.v.) on Day 1. Tumours on the left flank were not irradiated.

Tumors were measured in two dimensions using calipers, and volume was calculated using the formula:

Tumor Volume (mm³)=w²×l/2, where w=width and l=length, in mm, of the tumor.

Tumor weight may be estimated with the assumption that 1 mg is equivalent to 1 mm³ of tumor volume.

Results

| | | |
|---|---|---|
| Coefficient of | IRRADIATED TUMORS | Day 20 |
| Drug interaction | CD2S-ADC | 0.94 |
| | Radiotherapy | (synergism) |
| | CD25-ADC + Radiotherapy | |

| | | |
|---|---|---|
| Coefficient of | NON-IRRADIATED TUMORS | Day 20 |
| Drug interaction | CD25-ADC | 0.91 |
| | Radiotherapy | (synergism) |
| | CD25-ADC + Radiotherapy | |

Conclusions

In the bilateral CT26 tumor model, combination of CD25-ADC with focal radiotherapy resulted in synergistic anti-tumor activity in both the irradiated and non-irradiated distal tumor (abscopal effect) and the combination significantly increased survival compared to the single treatments.

### Example 10

In vivo studies of combined treatment with surADCx25 and radiotherapy; testing the effect of order of administration

Methodology

The base methodology is as described for Example 9.

In this part of study, and as noted above, there were two parallel groups of animals:
A) Animals where a single 0.5 mg/kg dose of surADCx25 (also described herein as “sur301”) was administered intravenously (i.v.) on Day 0, while tumor-targeted radiation treatment was administered only to the tumour on the right flank once on Day 1 at 5 Gy; and
B) Animals where a tumor-targeted radiation treatment was administered only to the tumour on the right flank once on Day 0 at 5 Gy, while a single 0.5 mg/kg dose of surADCx25 (also described herein as “sur301”) was administered intravenously (i.v.) on Day 1.

Results

See FIGS. 20 & 21 for full results.

### CONCLUSIONS

Sequential administration of CD25-ADC followed by radiotherapy resulted in superior anti-tumor activity compared to the reverse order of administration (radiotherapy first, followed by CD25-ADC), suggesting prior Treg depletion allows for optimal anti-tumor activity mediated by radiotherapy.

1. A method of inducing or enhancing an immune response against a cancer in a subject, the method comprising administering to the subject an effective amount of an anti-CD25 antibody drug conjugate (ADC) in combination with radiotherapy, wherein the treatment comprises administering the anti-CD25 ADC before the radiotherapy.

2-4. (canceled)

5. A method for treating a cancer in a subject, the method comprising administering to the subject an effective amount of an anti-CD25 antibody drug conjugate (ADC) in combination with radiotherapy, wherein the treatment comprises administering the anti-CD25 ADC before the radiotherapy.

6-8. (canceled)

9. The method of claim 5, wherein the CD25-ADC and radiotherapy are administered on the same day.

10-30. (canceled)

31. The method of claim 5, wherein:
(i) the immune-suppressive activity of a population of regulatory immune cells in the subject is reduced by at least 90% before the radiotherapy is administered; and/or
(ii) the size of a population of regulatory immune cells in the subject is reduced by at least 90% before the radiotherapy is administered.

32. The method of claim 5, wherein the regulatory immune cells are Treg cells.

33. The method of claim 5, wherein the subject:
a) has a disorder or has been determined to have a disorder;
b) has been determined to have a cancer which expresses CD25 or CD25+ tumour-associated non-tumour cells, such as CD25+ infiltrating cells; or
c) is radiosensitive.

34-35. (canceled)

35. The method of claim 5, wherein:
a) the radiotherapy is focal radiotherapy;
b) the radiotherapy is tumour targeted; or
c) the radiotherapy is selected from the group consisting of: external beam radiotherapy, stereotactic radiation therapy, Intensity-Modulated Radiation Therapy, particle therapy, brachytherapy, delivery of radioisotopes, intraoperative radiotherapy, Auger therapy, Volumetric modulated arc therapy, Virtual simulation, 3-dimensional conformal radiation therapy, and intensity-modulated radiation therapy.

36-38. (canceled)

39. The method of claim 5, wherein the radiotherapy is sub-therapeutic dose for treatment of the disorder with radiotherapy alone.

40. The method of claim 5, wherein the total radiotherapy dose is no greater than 40 Gy.

41-48. (canceled)

49. The method of claim 5, wherein the radiotherapy is administered as a single dose.

50. The method of claim 5, wherein the radiotherapy is administered as fractionated doses.

51. The method of claim 50, wherein each fractionated dose is no greater than 20 Gy.

52-60. (canceled)

61. The method of claim 50, wherein the radiotherapy is administered in two, three, four, five, six, eight, or ten fractionated doses.

62-67. (canceled)

68. The method of claim 50, wherein the fractionated doses are administered once daily (QD), once every other day (Q2D), once every third day (Q3D) or once weekly (QW).

69-73. (canceled)

74. The method of claim 5, wherein the cancer comprises a solid tumor, and wherein the treatment induces or enhances an immune response against the at least one of the solid tumors.

75-80. (canceled)

81. The method of claim 74, wherein the solid tumour comprises CD25−ve neoplastic cells, or is associated with CD25+ve infiltrating cells.

82. (canceled)

83. The method of claim 74, wherein the solid tumour is selected from the group consisting of pancreatic cancer, breast cancer, colorectal cancer, gastric and oesophageal cancer, melanoma, non-small cell lung cancer, ovarian cancer, hepatocellular carcinoma, renal cell carcinoma, bladder, and head and neck cancer.

84-86. (canceled)

87. The method of claim 73, wherein the cancer is selected from:
Hodgkin's Lymphoma;
non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and
leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

88. The method of claim 73, wherein the cancer is associated with elevated levels of regulatory immune cells.

89. The method of claim 5, wherein the CD25-ADC is administered in combination with a checkpoint inhibitor or other immunostimulatory agent.

90. (canceled)

91. The method of claim 89, wherein the checkpoint inhibitor is:
a) a PD1 antagonist selected from pembrolizumab, nivolumab, MEDI0680, PDR001 (spartalizumab), Camrelizumab, AUNP12, Pidilizumab Cemiplimab (REGN-2810), AMP 224, BGB-A317 (Tislelizumab), and BGB-108;
b) a PD-L1 antagonist selected from atezolizumab (Tecentriq), BMS-936559/MDX-1105, durvalumab/MEDI4736, and MSB0010718C (Avelumab);
c) a Glucocorticoid-Induced TNFR-Related (GITR) protein agonist selected from MEDI1873, TRX518, GWN323, MK-1248, MK 4166, BMS-986156 and INCAGN1876
d) an OX40 agonist selected from MEDI0562, MEDI6383, MOXR0916, RG7888, OX40mAb24, INCAGN1949, GSK3174998, and PF-0451860; or
e) a CTLA-4 antagonist selected from ipilimumab and Tremelimumab.

92-101. (canceled)

102. The method of claim 5, wherein the CD25-ADC comprises a PBD drug moiety, optionally wherein the CD25-ADC is a conjugate of formula:
L-(D^{L})ₚ,
where D^{L} is of formula I or II: 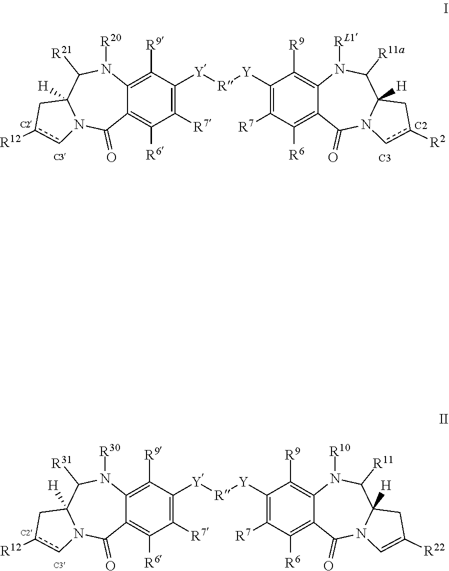
wherein:
L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 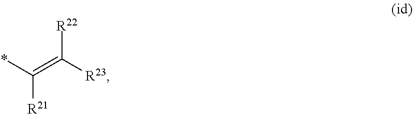
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 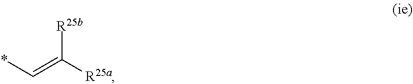
wherein one of R^{25a} and R^{25b} is H and the other is selected from: phenyl, which phenyl is optionally substituted by a group selected from halo, methyl, methoxy; pyridyl; and thiophenyl; and 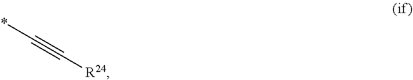
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 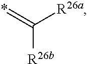
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
wherein, if D^{L} is of formula I:
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 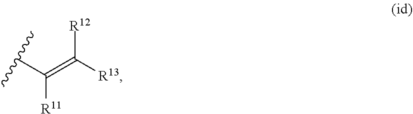
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 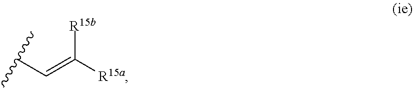
wherein one of R^{15a} and R^{15b} is H and the other is selected from: phenyl, which phenyl is optionally substituted by a group selected from halo, methyl, methoxy; pyridyl; and thiophenyl; and 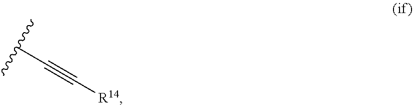
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 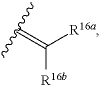
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
wherein, if D^{L} is of formula I:
R²² is of formula IIIa formula IIIb or formula IIIc: 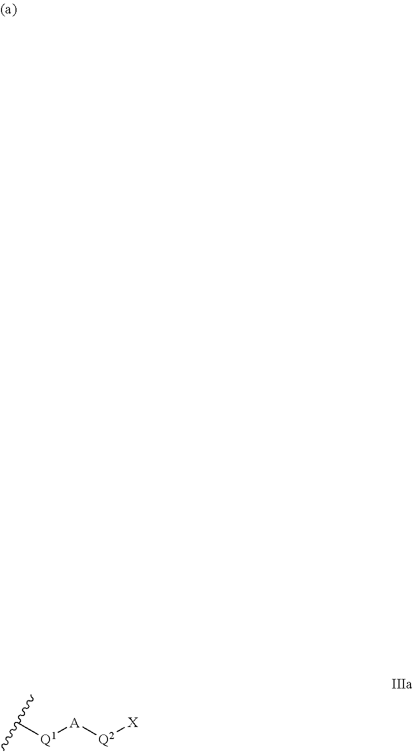
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 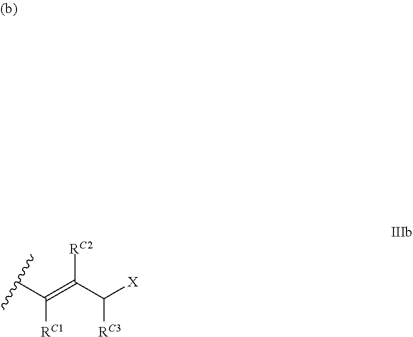
where;
R^{C1}, R^{C2} and R^{C3} are independently selected from H and unsubstituted C₁₋₂ alkyl; 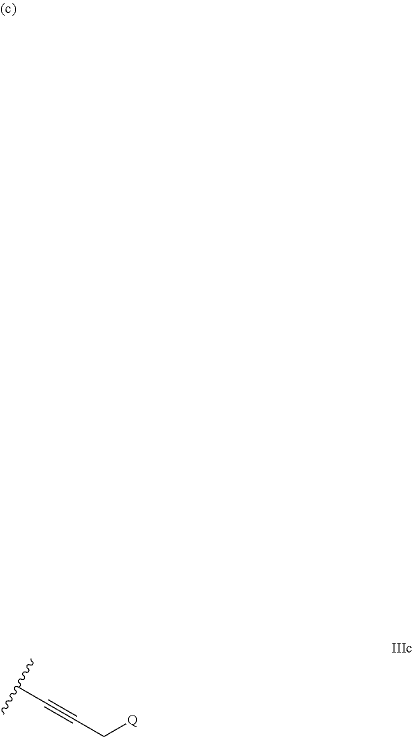
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′} NHNH—R^{L2′}, CONHNH—R^{L2′}, 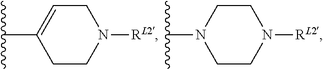
NR^{N}R^{L2′} wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

103-104. (canceled)

105. The method of claim 5, wherein the anti-CD25-ADC is Camidanlumab Tesirine.

106-109. (canceled)

110. The method of claim 5, wherein the anti-CD25 ADC is administered 1 to 21 days before the radiotherapy.

111. The method of claim 5, wherein the anti-CD25 ADC is administered 1 day, 2 days, 3 days, 4 days, 5 days, 6 days, or 7 days before the radiotherapy.

112. The method of claim 5, wherein the anti-CD25 ADC is administered 1 hour, 2 hours, 6 hours, 12 hours, or 24 hours before the radiotherapy.


SEQUENCE LISTING (57 sequences)

SEQ ID NO: 1 - protein, 127 aa, Artificial Sequence
  AB12 VH
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Artificial Sequence
  AB12 VL
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Artificial Sequence
  VH CDR1
RYIIN

SEQ ID NO: 4 - protein, 17 aa, Artificial Sequence
  VH CDR2
RIIPILGVENYAQKFQG

SEQ ID NO: 5 - protein, 6 aa, Artificial Sequence
  VH CDR3
KDWFDY

SEQ ID NO: 6 - protein, 11 aa, Artificial Sequence
  VL CDR1
RASQSVSSYLA

SEQ ID NO: 7 - protein, 7 aa, Artificial Sequence
  VL CDR2
GASSRAT

SEQ ID NO: 8 - protein, 9 aa, Artificial Sequence
  VL CDR3
QQYGSSPLT

SEQ ID NO: 9 - protein, 7 aa, Artificial Sequence
  Camrelizumab
FSESTNS

SEQ ID NO: 10 - protein, 7 aa, Artificial Sequence
  Camrelizumab
SNTSESF

SEQ ID NO: 11 - protein, 14 aa, Artificial Sequence
  Camrelizumab
FRVTQLAPKAQIKQ

SEQ ID NO: 12 - protein, 7 aa, Artificial Sequence
  Camrelizumab
SNTSESF

SEQ ID NO: 13 - protein, 22 aa, Artificial Sequence
  Camrelizumab
SNTSESFKFRVTQLAPKAQIKE

SEQ ID NO: 14 - protein, 5 aa, Artificial Sequence
  BMS-936559/MDS-1105 (VH CDR1)
DYGFS

SEQ ID NO: 15 - protein, 17 aa, Artificial Sequence
  BMS-936559/MDS-1105 (VH CDR2)
WITAYNGNTNYAQKLQG

SEQ ID NO: 16 - protein, 8 aa, Artificial Sequence
  BMS-936559/MDS-1105 (VH CDR3)
DYFYGMDV

SEQ ID NO: 17 - protein, 11 aa, Artificial Sequence
  BMS-936559/MDS-1105 (VL CDR1)
RASQSVSSYLV

SEQ ID NO: 18 - protein, 7 aa, Artificial Sequence
  BMS-936559/MDS-1105 (VL CDR2)
DASNRAT

SEQ ID NO: 19 - protein, 9 aa, Artificial Sequence
  BMS-936559/MDS-1105 (VL CDR3)
QQRSNWPRT

SEQ ID NO: 20 - protein, 5 aa, Artificial Sequence
  BMS-936559 / MDX-1105 (VH CDR1)
TYAIS

SEQ ID NO: 21 - protein, 17 aa, Artificial Sequence
  BMS-936559 / MDX-1105 (VH CDR2)
GIIPIFGKAHYAQKFQG

SEQ ID NO: 22 - protein, 14 aa, Artificial Sequence
  BMS-936559 / MDX-1105 (VH CDR3)
KFHFVSGSPFGMDV

SEQ ID NO: 23 - protein, 11 aa, Artificial Sequence
  BMS-936559 / MDX-1105 (VL CDR1)
RASQSVSSYLA

SEQ ID NO: 24 - protein, 8 aa, Artificial Sequence
  BMS-936559 / MDX-1105 (VL CDR3)
QQRSNWPT

SEQ ID NO: 25 - protein, 5 aa, Artificial Sequence
  BMS-936559 / MDX-1105 (VH CDR1)
SYDVH

SEQ ID NO: 26 - protein, 17 aa, Artificial Sequence
  BMS-936559 / MDX-1105 (VH CDR2)
WLHADTGITKFSQKFQG

SEQ ID NO: 27 - protein, 9 aa, Artificial Sequence
  BMS-936559 / MDX-1105 (VH CDR3)
ERIQLWFDY

SEQ ID NO: 28 - protein, 11 aa, Artificial Sequence
  BMS-936559 / MDX-1105 (VL CDR1)
RASQGISSWLA

SEQ ID NO: 29 - protein, 7 aa, Artificial Sequence
  BMS-936559 / MDX-1105 (VL CDR2)
AASSLQS

SEQ ID NO: 30 - protein, 9 aa, Artificial Sequence
  BMS-936559 / MDX-1105 (VL CDR3)
QQYNSYPYT

SEQ ID NO: 31 - protein, 121 aa, Artificial Sequence
  durvalumab/MEDI4736 (VH)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 32 - protein, 108 aa, Artificial Sequence
  durvalumab/MEDI4736 (VL)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 33 - protein, 107 aa, Artificial Sequence
  TRX518, a humanized agylcosylated (Fc disabled) IgG1 anti-GITR mAb (VL)
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 34 - protein, 118 aa, Artificial Sequence
  TRX518, a humanized agylcosylated (Fc disabled) IgG1 anti-GITR mAb (VH)
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVS

SEQ ID NO: 35 - protein, 118 aa, Artificial Sequence
  TRX518, a humanized agylcosylated (Fc disabled) IgG1 anti-GITR mAb (VH)
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVS

SEQ ID NO: 36 - protein, 450 aa, Artificial Sequence
  MEDI0562 (aka Tavolixizumab, Tavolimab) (HC)
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 210 aa, Artificial Sequence
  MEDI0562 (aka Tavolixizumab, Tavolimab) (LC)
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 38 - protein, 402 aa, Artificial Sequence
  MEDI6383 (Efizonerimod alfa)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 39 - protein, 121 aa, Artificial Sequence
  OX40mAb24 (9B12) VH
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 40 - protein, 107 aa, Artificial Sequence
  OX40mAb24 (9B12) VL
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 41 - protein, 5 aa, Artificial Sequence
  INCAGN1949 VH CDR1
GSAMH

SEQ ID NO: 42 - protein, 19 aa, Artificial Sequence
  INCAGN1949 VH CDR2
RIRSKANSYATAYAASVKG

SEQ ID NO: 43 - protein, 10 aa, Artificial Sequence
  INCAGN1949 VH CDR3
GIYDSSGYDY

SEQ ID NO: 44 - protein, 16 aa, Artificial Sequence
  INCAGN1949 VL CDR1
RSSQSLLHSNGYNYLD

SEQ ID NO: 45 - protein, 7 aa, Artificial Sequence
  INCAGN1949 VL CDR2
LGSNRAS

SEQ ID NO: 46 - protein, 9 aa, Artificial Sequence
  INCAGN1949 VL CDR3
MQALQTPLT

SEQ ID NO: 47 - protein, 121 aa, Artificial Sequence
  INCAGN1949 VH
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTSGIYDSSGYDYWGQGTLVTVSS

SEQ ID NO: 48 - protein, 112 aa, Artificial Sequence
  INCAGN1949 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 49 - protein, 167 aa, Artificial Sequence
  Tremelimumab  VH
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 50 - protein, 139 aa, Artificial Sequence
  Tremelimumab  VL
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 51 - protein, 223 aa, Homo sapiens
  misc_feature: CTLA4 GenBank AAL07473.1
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 52 - DNA, 2025 bp, Homo sapiens
  misc_feature: CTLA4 (CTLA4) mRNA, complete cds GenBank AF414120.1
cttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttaaaaaaaaaaaaaaaaaaaaaaaaaaaaaaaaaaaaaaaaaaaaaaaaaaaaaaaaaa

SEQ ID NO: 53 - protein, 223 aa, Homo sapiens; identical to SEQ ID NO: 51
  misc_feature: Cytotoxic T-lymphocyte protein 4 UniProt P16410

SEQ ID NO: 54 - protein, 241 aa, Homo sapiens
  misc_feature: glucocorticoid-induced TNFR-related protein GenBank AAD22635.1
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 55 - protein, 241 aa, Homo sapiens; identical to SEQ ID NO: 54
  misc_feature: TNR18_HUMAN Tumor necrosis factor receptor superfamily member 18 UniProt Q9Y5U5

SEQ ID NO: 56 - protein, 277 aa, Homo sapiens
  misc_feature: OX40 homologue GenBank CAA53576.1
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 57 - protein, 277 aa, Homo sapiens; identical to SEQ ID NO: 56
  misc_feature: TNR4_HUMAN Tumor necrosis factor receptor superfamily member 4 UniProt P43489